SEQUENCE LISTING (49 sequences)

SEQ ID NO: 1 - protein, 121 aa, Artificial Sequence
  Description of Artificial Sequence Synthetic polypeptide
QVQLQQSGAELVKPGASVKISCKASGYAFSSYWMNWVKQRPGEGLEWIGQIYPGDDDTNYNGKFKGKATLTVDKSSTTAYMQLSSLTSEDSAVYFCAGSLMVTTGAPFDYWGQGTTLTVSS

SEQ ID NO: 2 - protein, 107 aa, Artificial Sequence
  Description of Artificial Sequence Synthetic polypeptide
DIVMTQSHKFMSTSVGDRVSITCKASQDVGIAVAWYQQKPGQSPKFLIYWASTRHTGVPDRFTGSGSGTDFTLTIRNVQSEDLSDYFCQQYRSYPLTFGSGTKLELK

SEQ ID NO: 3 - protein, 121 aa, Artificial Sequence
  Description of Artificial Sequence Synthetic polypeptide
QVQLVQSGAEVKKPGSSVKVSCKASGYAFSSYWMNWVRQAPGQGLEWIGQIYPGDDDTNYNGKFQGRVTITVDKSTTTAYMELSSLRSEDTAVYFCAGSLMVTTGAPFDYWGQGTTVTVSS

SEQ ID NO: 4 - protein, 107 aa, Artificial Sequence
  Description of Artificial Sequence Synthetic polypeptide
DIQMTQSPSSLSASVGDRVTITCKASQDVGIAVAWYQQKPGKAPKFLIYWASTRHTGVPDRFSGSGSGTDFTLTISSLQPEDFATYFCQQYRSYPLTFGQGTKLEIK

SEQ ID NO: 5 - protein, 121 aa, Artificial Sequence
  Description of Artificial Sequence Synthetic polypeptide
QVQLVQSGAEVKKPGSSVKVSCKASGYAFSSYWMNWVRQAPGQGLEWIGQIYPGDDDTNYNAKFQGRVTITVDKSTTTAYMELSSLRSEDTAVYFCAGSLMVTTGAPFDYWGQGTTVTVSS

SEQ ID NO: 6 - protein, 107 aa, Artificial Sequence
  Description of Artificial Sequence Synthetic polypeptide
DIQMTQSPSSLSASVGDRVTITCKASQDVGIAVAWYQQKPGKAPKFLIYYASHRGWGVPDRFSGSGSGTDFTLTISSLQPEDFATYFCQQYRSYPLTFGQGTKLEIK

SEQ ID NO: 7 - protein, 450 aa, Artificial Sequence
  Description of Artificial Sequence Synthetic polypeptide
QVQLVQSGAEVKKPGSSVKVSCKASGYAFSSYWMNWVRQAPGQGLEWIGQIYPGDDDTNYNAKFQGRVTITVDKSTTTAYMELSSLRSEDTAVYFCAGSLMVTTGAPFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 214 aa, Artificial Sequence
  Description of Artificial Sequence Synthetic polypeptide
DIQMTQSPSSLSASVGDRVTITCKASQDVGIAVAWYQQKPGKAPKFLIYYASHRGWGVPDRFSGSGSGTDFTLTISSLQPEDFATYFCQQYRSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 450 aa, Artificial Sequence
  Description of Artificial Sequence Synthetic polypeptide
QVQLVQSGAEVKKPGSSVKVSCKASGYAFSSYWMNWVRQAPGQGLEWIGQIYPGDDDTNYNAKFQGRVTITVDKSTTTAYMELSSLRSEDTAVYFCAGSLMVTTGAPFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 10 - protein, 450 aa, Artificial Sequence
  Description of Artificial Sequence Synthetic polypeptide
QVQLVQSGAEVKKPGSSVKVSCKASGYAFSSYWMNWVRQAPGQGLEWIGQIYPGDDDTNYNAKFQGRVTITVDKSTTTAYMELSSLRSEDTAVYFCAGSLMVTTGAPFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 11 aa, Artificial Sequence
  Description of Artificial Sequence Synthetic peptide
KASQDVGIAVA

SEQ ID NO: 12 - protein, 7 aa, Artificial Sequence
  Description of Artificial Sequence Synthetic peptide
WASTRHT

SEQ ID NO: 13 - protein, 9 aa, Artificial Sequence
  Description of Artificial Sequence Synthetic peptide
QQYRSYPLT

SEQ ID NO: 14 - protein, 5 aa, Artificial Sequence
  Description of Artificial Sequence Synthetic peptide
SYWMN

SEQ ID NO: 15 - protein, 17 aa, Artificial Sequence
  Description of Artificial Sequence Synthetic peptide
QIYPGDDDTNYNGKFKG

SEQ ID NO: 16 - protein, 12 aa, Artificial Sequence
  Description of Artificial Sequence Synthetic peptide
SLMVTTGAPFDY

SEQ ID NO: 17 - protein, 17 aa, Artificial Sequence
  Description of Artificial Sequence Synthetic peptide
QIYPGDDDTNYNGKFQG

SEQ ID NO: 18 - protein, 7 aa, Artificial Sequence
  Description of Artificial Sequence Synthetic peptide
YASHRGW

SEQ ID NO: 19 - protein, 17 aa, Artificial Sequence
  Description of Artificial Sequence Synthetic peptide
QIYPGDDDTNYNAKFQG

SEQ ID NO: 20 - protein, 7 aa, Artificial Sequence
  Description of Artificial Sequence Synthetic peptide
SQDVGIA

SEQ ID NO: 21 - protein, 3 aa, Artificial Sequence
  Description of Artificial Sequence Synthetic peptide
WAS

SEQ ID NO: 22 - protein, 7 aa, Artificial Sequence
  Description of Artificial Sequence Synthetic peptide
YRSYPLT

SEQ ID NO: 23 - protein, 7 aa, Artificial Sequence
  Description of Artificial Sequence Synthetic peptide
GYAFSSY

SEQ ID NO: 24 - protein, 6 aa, Artificial Sequence
  Description of Artificial Sequence Synthetic peptide
YPGDDD

SEQ ID NO: 25 - protein, 12 aa, Artificial Sequence
  Description of Artificial Sequence Synthetic peptide
SLMVTTGAPFDY

SEQ ID NO: 26 - protein, 3 aa, Artificial Sequence
  Description of Artificial Sequence Synthetic peptide
YAS

SEQ ID NO: 27 - protein, 6 aa, Artificial Sequence
  Description of Artificial Sequence Synthetic peptide
QDVGIA

SEQ ID NO: 28 - protein, 9 aa, Artificial Sequence
  Description of Artificial Sequence Synthetic peptide
QQYRSYPLT

SEQ ID NO: 29 - protein, 8 aa, Artificial Sequence
  Description of Artificial Sequence Synthetic peptide
GYAFSSYW

SEQ ID NO: 30 - protein, 8 aa, Artificial Sequence
  Description of Artificial Sequence Synthetic peptide
IYPGDDDT

SEQ ID NO: 31 - protein, 14 aa, Artificial Sequence
  Description of Artificial Sequence Synthetic peptide
AGSLMVTTGAPFDY

SEQ ID NO: 32 - protein, 638 aa, Homo sapiens
MILFKQATYFISLFATVSCGCLTQLYENAFFRGGDVASMYTPNAQYCQMRCTFHPRCLLFSFLPASSINDMEKRFGCFLKDSVTGTLPKVHRTGAVSGHSLKQCGHQISACHRDIYKGVDMRGVNFNVSKVSSVEECQKRCTNNIRCQFFSYATQTFHKAEYRNNCLLKYSPGGTPTAIKVLSNVESGFSLKPCALSEIGCHMNIFQHLAFSDVDVARVLTPDAFVCRTICTYHPNCLFFTFYTNVWKIESQRNVCLLKTSESGTPSSSTPQENTISGYSLLTCKRTLPEPCHSKIYPGVDFGGEELNVTFVKGVNVCQETCTKMIRCQFFTYSLLPEDCKEEKCKCFLRLSMDGSPTRIAYGTQGSSGYSLRLCNTGDNSVCTTKTSTRIVGGTNSSWGEWPWQVSLQVKLTAQRHLCGGSLIGHQWVLTAAHCFDGLPLQDVWRIYSGILNLSDITKDTPFSQIKEIIIHQNYKVSEGNHDIALIKLQAPLNYTEFQKPICLPSKGDTSTIYTNCWVTGWGFSKEKGEIQNILQKVNIPLVTNEECQKRYQDYKITQRMVCAGYKEGGKDACKGDSGGPLVCKHNGMWRLVGITSWGEGCARREQPGVYTKVAEYMDWILEKTQSSDGKAQMQSPA

SEQ ID NO: 33 - DNA, 363 bp, Artificial Sequence
  Description of Artificial Sequence Synthetic polynucleotide
caggttcagctgcagcagtctggggctgagctggtgaagcctggggcctcagtgaagatttcctgcaaagcttctggctacgcattcagtagctactggatgaactgggtgaagcagaggcctggagagggtcttgagtggattggacagatttatcctggagatgatgatactaactacaacggaaagttcaagggcaaggccacactgactgtagacaaatcctccaccacagcctacatgcagctcagcagcctgacctctgaggactctgcggtctatttctgtgcaggatctttgatggttacgacgggggccccctttgactactggggccaaggcaccactctcacagtctcctca

SEQ ID NO: 34 - DNA, 321 bp, Artificial Sequence
  Description of Artificial Sequence Synthetic polynucleotide
gacattgtgatgacccagtctcacaaattcatgtccacatcagtaggagacagggtcagcatcacctgcaaggccagtcaggatgtgggtattgctgttgcctggtatcaacagaaaccagggcaatctcctaaatttctgatttactgggcatccacccggcacactggagtccctgatcgcttcacaggcagtggatctgggacagatttcactctcaccattaggaatgtgcagtctgaagacttgtcagattatttctgtcagcaatataggagctatcctctcacgttcggctcggggacaaagttggaactaaaa

SEQ ID NO: 35 - protein, 5 aa, Artificial Sequence
  Description of Artificial Sequence Synthetic peptide
GGGGS

SEQ ID NO: 36 - protein, 5 aa, Artificial Sequence
  Description of Artificial Sequence Synthetic peptide
GGGGA

SEQ ID NO: 37 - protein, 2 aa, Unknown
  Description of Unknown MAb4-YTE epitope 1 sequence
KR

SEQ ID NO: 38 - protein, 13 aa, Unknown
  Description of Unknown MAb4-YTE epitope 2 sequence
KHNGMWRLVGITS

SEQ ID NO: 39 - protein, 9 aa, Unknown
  Description of Unknown MAb4-YTE epitope 3 sequence
YMDWILEKT

SEQ ID NO: 40 - protein, 13 aa, Unknown
  Description of Unknown DX2930 epitope 1 sequence
HCFDGLPLQDVWR

SEQ ID NO: 41 - protein, 8 aa, Unknown
  Description of Unknown DX2930 epitope 2 sequence
YKVSEGNH

SEQ ID NO: 42 - protein, 6 aa, Unknown
  Description of Unknown DX2930 epitope 3 sequence
YKITQR

SEQ ID NO: 43 - protein, 10 aa, Artificial Sequence
  Description of Artificial Sequence Synthetic peptide
GYAFSSYWMN

SEQ ID NO: 44 - protein, 10 aa, Artificial Sequence
  Description of Artificial Sequence Synthetic peptide
QIYPGDDDTN

SEQ ID NO: 45 - protein, 638 aa, Macaca fascicularis
MILFKQATYFISLFATVSCGCLTQLYENAFFRGGDVASMYTPTAQHCQMMCTFHPRCLLFSFLPASSINDMEKRFGCFLKDSVTGTLPKVRRAGAISGHSLKQCGHQISACHRDIYKGIDMRGVNFNVSKVSSVEECQKRCTNNIRCQFFSYATQTFHNAEYRNTCLLKHSPGGTPTTIKVLNNVESGFSLKPCALSEIGCHMNIFQHLAFSDVDVARVLAPDAFVCRTICTYHPSCLFFTFYTNAWKIESQRNVCFLKTSESGTPSSSTPQENTTSGYSLLTCKRTLPEPCHSKIYPGVDFGGEELNVTFVKGVNVCQETCTKMIRCQFFTYSLLPEDCKEEKCKCFLRLSSDGSPTRITYGTQGSSGYSLRLCNTGDSSVCTTKTSSRIVGGTNSSWGEWPWQVSLQVKLMAQRHLCGGSLIGHQWVLTAAHCFDGLPLPDVWRIYSGILNLSDITKETPFSQIKEIIIHQNYRISEGNHDIALIKLQAPLNYTEFQKPICLPSKGDTNTIYTNCWVTGWGFSKEKGEIQDILQKVNIPLVTNEECQKRYQDYKITQRMVCAGYKEGGKDACKGDSGGPLACKHNGMWRLVGITSWGEGCARREQPGVYTKVAEYMDWILEKTQSSDGNARMQAPA

SEQ ID NO: 46 - protein, 629 aa, Oryctolagus cuniculus
MIPFRQAAYFVCLFTTVSCGCLTQLYKNAFFRGGDVTSMYSPNAQHCQMMCTFHPRCLFFSFLPASSTNDAQKRFGCFLKDSVTGTLPRVHQTNAISGHSLKQCGHQINACHRATYEGIDMRGTNFNISKVQSVEECQKQCTDNIHCQFFTYATQTFYNAEHLNNCILKKSATGTPSSIKLLENVVSGFSLKPCALSESGCHMNLFQHLAFSDVDVARVLTPDAFVCRTICTYHSNCLFFTFYTNEWDTESQRNACFLKTSESGTPSFPTPQENTISGYSLLTCKKTLPEPCHFKIYSGVDFGGEELNVTFVKGVTLCQETCTKTIRCQFFTYSLLPEDCKGEKCRCSLRLSLDGSPTSITHGTQKSSGYSLRLCKTENGSVCTTKINARIVGGSNSSRGEWPWQVSLQVKLAAQSHVCGGSIIGHQWVLTAAHCFDGLPFPEIWRIYGGILYLSEVTKETAFSQIKEIIIHPKYKISETGHDIALIQLQAPLNDTDIQKPICLPSKDDTNAIYTNCWVTGWGFTKEKGEIQNILQKANIPLVTNEECQKSYRDHAITKQMVCAGYKEGGKDACKGDSGGPLVCKHNNIWLLVGITSWGEGCARREQPGVYTKVAEYVDWILQKMQEKD

SEQ ID NO: 47 - protein, 638 aa, Rattus norvegicus
MILFKQVGYFVSLFATVSCGCLSQLYANTFFRGGDLAAIYTPDAQHCQKMCTFHPRCLLFSFLAVSPTKETDKRFGCFMKESITGTLPRIHRTGAISGHSLKQCGHQLSACHQDIYEGLDMRGSNFNISKTDSIEECQKLCTNNIHCQFFTYATKAFHRPEYRKSCLLKRSSSGTPTSIKPVDNLVSGFSLKSCALSEIGCPMDIFQHFAFADLNVSQVVTPDAFVCRTVCTFHPNCLFFTFYTNEWETESQRNVCFLKTSKSGRPSPPIIQENAVSGYSLFTCRKARPEPCHFKIYSGVAFEGEELNATFVQGADACQETCTKTIRCQFFTYSLLPQDCKAEGCKCSLRLSTDGSPTRITYEAQGSSGYSLRLCKVVESSDCTTKINARIVGGTNSSLGEWPWQVSLQVKLVSQNHMCGGSIIGRQWILTAAHCFDGIPYPDVWRIYGGILNLSEITNKTPFSSIKELIIHQKYKMSEGSYDIALIKLQTPLNYTEFQKPICLPSKADTNTIYTNCWVTGWGYTKERGETQNILQKATIPLVPNEECQKKYRDYVITKQMICAGYKEGGIDACKGDSGGPLVCKHSGRWQLVGITSWGEGCARKEQPGVYTKVAEYIDWILEKIQSSKERALETSPA

SEQ ID NO: 48 - DNA, 1350 bp, Artificial Sequence
  Description of Artificial Sequence Synthetic polynucleotide
caggtgcagctggtgcagtctggcgccgaagtgaagaaacctggctcctccgtgaaggtgtcctgcaaggcctccggctatgcattcagtagctactggatgaactgggtgaggcaggctcctggacagggtcttgagtggattggacagatttatcctggagatgacgatactaactacaatgccaagttccaggggagggtcaccatcaccgtggacaaatccacaactaccgcctacatggaactcagcagcctacgctctgaggacacagccgtctatttctgtgctggctccctgatggttactaccggcgccccttttgactactggggccagggaaccacggtcaccgtctcctcagcctccaccaagggcccatcggtcttccccctggcaccctcttctaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctctacatcacccgggaacctgaggtcacatgcgtggtggtggacgtgagccacgaagaccctgaggtcaagttcaactggtacgtggacggcgtggaggtgcataatgccaagacaaagccgagagaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctcccggg

SEQ ID NO: 49 - DNA, 642 bp, Artificial Sequence
  Description of Artificial Sequence Synthetic polynucleotide
gacatccagatgacccagtctccatcctccctgtctgcatctgtaggagacagagtcaccatcacttgcaaagcaagtcaggacgtgggaatcgctgtcgcctggtatcagcagaaaccagggaaagcccctaagttcctgatctattacgcatcccaccgcggatggggcgtgcctgacaggttcagtggcagtggatctgggacagatttcactctcaccatcagcagtctgcaacctgaagattttgcaacttatttctgtcaacagtaccggtcttatcccctgacctttggccaagggaccaagctggaaatcaaacgaactgtggctgcaccatctgtcttcatcttcccgccatctgatgagcagcttaagtccggaactgctagcgttgtgtgcctgctgaataacttctatcccagagaggccaaagtacagtggaaggtggataacgccctccaatcgggaaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

(19) United States
(12) Patent Application Publication
(10) Pub. No.: US 2024/0218079 A1
(43) Pub. Date: Jul. 4, 2024

(54) PLASMA KALLIKREIN ANTIBODIES AND USES THEREOF

(71) Applicant: Astria Therapeutics, Inc., Boston, MA (US)

(72) Inventors: Vahe Bedian, Ashburnham, MA (US); Pradeep Bista, Arlington, MA (US); Peter Evan Harwin, Dorado, PR (US); Tomas Kiselak, Dorado, PR (US); Jonathan Violin, Dover, MA (US)

(21) Appl. No.: 18/274,511

(22) PCT Filed: Jan. 28, 2022
(86) PCT No.: PCT/US2022/014242

§ 371 Date: Jul. 27, 2023

Related U.S. Application Data
(60) Provisional application No. 63/262,838, filed on Oct. 21, 2021.
(60) Provisional application No. 63/220,194, filed on Jul. 9, 2021.
(60) Provisional application No. 63/159,323, filed on Mar. 10, 2021.
(60) Provisional application No. 63/142,748, filed on Jan. 28, 2021.

(51) Int. Cl. C07K 16/40; A61K 39/00

(52) U.S. Cl. CPC C07K 16/40; A61K 2039/505; C07K 2317/14; C07K 2317/24; C07K 2317/33; C07K 2317/34; C07K 2317/565; C07K 2317/76; C07K 2317/92; C07K 2317/94

(57) ABSTRACT

Antibodies and compositions containing such antibodies or antigen binding fragments thereof that bind plasma kallikrein are provided herein. Also provided are methods of making and using such antibodies or antigen binding fragments thereof, such as in the treatment of hereditary angioedema or bradykinin dependent edema.

### CROSS REFERENCE TO RELATED APPLICATIONS

This application claims priority to and the benefit of U.S. Provisional Patent Application No. 63/142,748, filed Jan. 28, 2021; U.S. Provisional Patent Application No. 63/159,323, filed Mar. 10, 2021; U.S. Provisional Patent Application No. 63/220,194, filed Jul. 9, 2021; and U.S. Provisional Patent Application No. 63/262,838, filed Oct. 21, 2021, the contents of each of which are hereby incorporated by reference in their entireties.

### FIELD OF THE INVENTION

The field of the invention is immunology, in particular therapeutic antibodies, and treatment of disease with those antibodies.

### BACKGROUND

Plasma kallikrein is a serine protease and a potential drug target for different inflammatory, cardiovascular, infectious (sepsis) and oncology diseases (Sainz I. M. et al., Thromb Haemost 98, 77-83, 2007). Activation of the plasma kallikrein amplifies intrinsic coagulation via its feedback activation of factor XII and enhances inflammation via the production of the proinflammatory nonapeptide bradykinin. As the primary kininogenase in the circulation, plasma kallikrein is largely responsible for the generation of bradykinin in the vasculature. A genetic deficiency in the C1-inhibitor protein (C1-INH), the major natural inhibitor of plasma kallikrein, leads to hereditary angioedema (HAE). Patients with HAE suffer from acute attacks of painful edema often precipitated by unknown triggers (Zuraw B. L. et al., N Engl J Med 359, 1027-1036, 2008). Through the use of pharmacological agents or genetic studies in animal models, the plasma kallikrein-kinin system (plasma KKS) has been implicated in various diseases. Plasma kallikrein binding proteins (e.g., antibodies, e.g., inhibitory antibodies) are useful therapeutic agents for a variety of diseases and conditions, e.g., diseases and conditions associated with the level of plasma kallikrein activity.

Despite the efforts made to date, there is a need for therapeutic antibodies that target plasma kallikrein.

### SUMMARY OF THE INVENTION

The invention described herein is based, in part, upon the discovery of high affinity binding proteins, e.g., antibodies and antigen binding fragments, that bind to plasma kallikrein, e.g. human plasma kallikrein. The antibodies have high specificity for plasma kallikrein, e.g. human plasma kallikrein. The antibodies have high selectivity for plasma kallikrein relative to prekallikrein, and lack significant off-target binding to trypsin and other serine proteases. These binding proteins may also have extended half-lives, e.g., extended half-lives in blood. Because of these and other features, the antibodies may be useful for treating a variety of plasma kallikrein-mediated disorders, such as hereditary angioedema and bradykinin dependent edema, and also facilitating the treatment of these disorders with lower doses and/or extending dosing intervals as compared to other antibodies.

In one aspect of the invention, disclosed herein are antibodies, including antigen binding fragments, that bind plasma kallikrein. The plasma kallikrein may be human plasma kallikrein, for example, human plasma kallikrein having the amino acid sequence as set forth in SEQ ID NO:32.

The antibodies and antigen binding fragments disclosed herein may include a heavy chain variable region (VH) comprising an HCDR1, an HCDR2, and an HCDR3; and a light chain variable region (VL) comprising an LCDR1, an LCDR2, and an LCDR3.

For example, the antibody or binding fragment thereof may have a VH wherein the HCDR1 comprises the amino acid sequence set forth in SEQ ID NO: 14, 23, 29, or 43; the HCDR2 comprises the amino acid sequence set forth in SEQ ID NO: 15, 17, 19, 24, 30, or 44; and the HCDR3 comprises the amino acid sequence set forth in SEQ ID NO: 16, 25, or 31; and a VL wherein the LCDR1 comprises the amino acid sequence set forth in SEQ ID NO: 11, 20, or 27; the LCDR2 comprises the amino acid sequence set forth in SEQ ID NO: 12, 18, 21, or 26; and the LCDR3 comprises the amino acid sequence set forth in of SEQ ID NO: 13, 22, or 28.

For example, the antibody or binding fragment thereof may have a VH wherein the HCDR1 comprises the amino acid sequence set forth in SEQ ID NO: 14; the HCDR2 comprises the amino acid sequence set forth in SEQ ID NO: 15, 17, or 19; and the HCDR3 comprises the amino acid sequence set forth in SEQ ID NO: 16; and a VL wherein the LCDR1 comprises the amino acid sequence set forth in SEQ ID NO: 11; the LCDR2 comprises the amino acid sequence set forth in SEQ ID NO: 12 or 18; and the LCDR3 comprises the amino acid sequence set forth in of SEQ ID NO: 13.

For example, the antibody or binding fragment thereof may have a VH wherein the HCDR1 comprises the amino acid sequence set forth in SEQ ID NO: 23; the HCDR2 comprises the amino acid sequence set forth in SEQ ID NO: 24; and the HCDR3 comprises the amino acid sequence set forth in SEQ ID NO: 25 and a VL wherein the LCDR1 comprises the amino acid sequence set forth in SEQ ID NO: 20; the LCDR2 comprises the amino acid sequence set forth in SEQ ID NO: 21 or 26; and the LCDR3 comprises the amino acid sequence set forth in SEQ ID NO: 22;

For example, the antibody or binding fragment thereof may have a VH wherein the HCDR1 comprises the amino acid sequence set forth in SEQ ID NO: 29; the HCDR2 comprises the amino acid sequence set forth in SEQ ID NO: 30; and the HCDR3 comprises the amino acid sequence set forth in SEQ ID NO: 31 and a VL wherein the LCDR1 comprises the amino acid sequence set forth in SEQ ID NO: 27; the LCDR2 comprises the amino acid sequence set forth in SEQ ID NO: 21 or 26; and the LCDR3 comprises the amino acid sequence set forth in SEQ ID NO: 28.

For example, the antibody or binding fragment thereof may have a VH wherein the HCDR1 comprises the amino acid sequence set forth in SEQ ID NO: 43; the HCDR2 comprises the amino acid sequence set forth in SEQ ID NO: 44; and the HCDR3 comprises the amino acid sequence set forth in SEQ ID NO: 25 and a VL wherein the LCDR1 comprises the amino acid sequence set forth in SEQ ID NO: 11; the LCDR2 comprises the amino acid sequence set forth in SEQ ID NO: 12 or 18 and the LCDR3 comprises the amino acid sequence set forth in SEQ ID NO: 28.

For example, the antibody or binding fragment thereof may have a VH wherein the HCDR1 comprises the amino acid sequence set forth in SEQ ID NO: 14; the HCDR2 comprises the amino acid sequence set forth in SEQ ID NO: 15, 17, or 19; and the HCDR3 comprises the amino acid sequence set forth in SEQ ID NO: 16; and a VL wherein the LCDR1 comprises the amino acid sequence set forth in SEQ ID NO: 11; the LCDR2 comprises the amino acid sequence set forth in SEQ ID NO: 12 or 18; and the LCDR3 comprises the amino acid sequence set forth in SEQ ID NO: 13.

For example, the antibody or binding fragment thereof may have a VH wherein the HCDR1 comprises the amino acid sequence set forth in SEQ ID NO: 14; the HCDR2 comprises the amino acid sequence set forth in SEQ ID NO: 19; and the HCDR3 comprises the amino acid sequence set forth in SEQ ID NO: 16; and a VL wherein the LCDR1 comprises the amino acid sequence set forth in SEQ ID NO: 11; the LCDR2 comprises the amino acid sequence set forth in SEQ ID NO: 18; and the LCDR3 comprises the amino acid sequence set forth in SEQ ID NO: 13.

For example, the antibody or binding fragment thereof may have a VH wherein the HCDR1 comprises the amino acid sequence set forth in SEQ ID NO: 14; the HCDR2 comprises the amino acid sequence set forth in SEQ ID NO: 15; and the HCDR3 comprises the amino acid sequence set forth in SEQ ID NO: 16; and a VL wherein the LCDR1 comprises the amino acid sequence set forth in SEQ ID NO: 11; the LCDR2 comprises the amino acid sequence set forth in SEQ ID NO: 12; and the LCDR3 comprises the amino acid sequence set forth in SEQ ID NO: 13.

For example, the antibody or binding fragment thereof may have a VH wherein the HCDR1 comprises the amino acid sequence set forth in SEQ ID NO: 14; the HCDR2 comprises the amino acid sequence set forth in SEQ ID NO: 17; and the HCDR3 comprises the amino acid sequence set forth in SEQ ID NO: 16; and a VL wherein the LCDR1 comprises the amino acid sequence set forth in SEQ ID NO: 11; the LCDR2 comprises the amino acid sequence set forth in SEQ ID NO: 12; and the LCDR3 comprises the amino acid sequence set forth in SEQ ID NO: 13.

For example, the antibody or binding fragment thereof may have a VH wherein the HCDR1 comprises the amino acid sequence set forth in SEQ ID NO: 23; the HCDR2 comprises the amino acid sequence set forth in SEQ ID NO: 24; and the HCDR3 comprises the amino acid sequence set forth in SEQ ID NO: 25; and a VL wherein the LCDR1 comprises the amino acid sequence set forth in SEQ ID NO: 20; the comprises the amino acid sequence set forth in SEQ ID NO: 26; and the LCDR3 comprises the amino acid sequence set forth in SEQ ID NO: 22.

For example, the antibody or binding fragment thereof may have a VH wherein the HCDR1 comprises the amino acid sequence set forth in SEQ ID NO: 29; the HCDR2 comprises the amino acid sequence set forth in SEQ ID NO: 30; and the HCDR3 comprises the amino acid sequence set forth in SEQ ID NO: 31; and a VL wherein the LCDR1 comprises the amino acid sequence set forth in SEQ ID NO: 27; the CDR2 comprises the amino acid sequence set forth in SEQ ID NO: 26; and the LCDR3 sequence comprises the amino acid sequence of SEQ ID NO: 28.

For example, the antibody or binding fragment thereof may have a VH wherein the HCDR1 comprises the amino acid sequence set forth in SEQ ID NO: 43; the HCDR2 comprises the amino acid sequence set forth in SEQ ID NO: 44; and the HCDR3 comprises the amino acid sequence set forth in SEQ ID NO: 25; and a VL wherein the LCDR1 comprises the amino acid sequence set forth in SEQ ID NO: 11; the LCDR2 comprises the amino acid sequence set forth in SEQ ID NO: 18 and the LCDR3 comprises the amino acid sequence set forth in SEQ ID NO: 28.

For example, the antibody or binding fragment thereof may have a VH wherein the HCDR1 comprises the amino acid sequence set forth in SEQ ID NO: 14 with up to 1, 2, or 3 amino acid substitutions to the amino acid sequence; the CDR2 comprises the amino acid sequence set forth in SEQ ID NO: 15, 17, or 19 with up to 1, 2, 3, 4, 5, 6, 7, 8, 9, 10, 11, 12, or 13 amino acid substitutions to the amino acid sequence; and the HCDR3 comprises the amino acid sequence set forth in SEQ ID NO: 16 with up to 1, 2, 3, 4, 5, 6, 7, 8, or 9 amino acid substitutions to the amino acid sequence; and a VL wherein the LCDR1 comprises the amino acid sequence set forth in SEQ ID NO: 11 with up to 1, 2, 3, 4, 5, 6, or 7 amino acid substitutions to the amino acid sequence; the LCDR2 comprises the amino acid sequence set forth in SEQ ID NO: 12 or 18 with up to 1, 2, 3, or 4 amino acid substitutions to the amino acid sequence; and the LCDR3 comprises the amino acid sequence set forth in SEQ ID NO: 13 with up to 1, 2, 3, or 4 amino acid substitutions to the amino acid sequence. In some of the antibodies or binding fragments, each of HCDR1, HCDR2, HCDR3, LCDR1, LCDR2, and LCDR3 comprises at most one amino acid substitution, while in others at most one CDR selected from HCDR1, HCDR2, or HCDR3 comprises a substitution and/or at most one CDR selected from LCDR1, LCDR2, and/or LCDR3 comprises a substitution.

The VL of the antibodies or binding fragments disclosed herein may include an amino acid sequence set forth in SEQ ID NOs: 2, 4, or 6, such as SEQ ID NO: 6. The VH of the antibodies or binding fragments disclosed herein may include an amino acid sequence set forth in SEQ ID NOs: 1, 3, or 5, such as SEQ ID NO: 5. The VH may include the amino acid sequence set forth in SEQ ID NO: 5 and the VL may include the amino acid sequence set forth in SEQ ID NO: 6. The VH may include the amino acid sequence set forth in SEQ ID NO: 1, 3, or 5 and the VL may include the amino acid sequence set forth in SEQ ID NOs: 2, 4, or 6.

The light chain of the antibodies or binding fragments disclosed herein may include an amino acid sequence as set forth in SEQ ID NO: 8. The heavy chain of the antibodies or binding fragments disclosed herein may include an amino acid sequence as set forth in SEQ ID NOs: 10, 9, or 7. For example, the light chain may include the amino acid sequence of SEQ ID NO: 8 and the heavy chain may include the amino acid sequence of SEQ ID NO: 7. For example, the light chain may include the amino acid sequence of SEQ ID NO: 8 and the heavy chain may include the amino acid sequence of SEQ ID NO: 9. For example, the light chain may include the amino acid sequence of SEQ ID NO: 8 and the heavy chain may include the amino acid sequence of SEQ ID NO: 10.

The antibody or antigen binding fragment may have a VH region with an amino acid sequence that is at least 70%, 80%, 85%, 86%, 87%, 88%, 89%, 90%, 91%, 92%, 93%, 94%, 95%, 96%, 97%, 98%, or 99% identical to the amino acid sequences set forth in SEQ ID NOs: 5, 3, or 1 and/or a VL region with an amino acid sequence that is at least 70%, 80%, 85%, 86%, 87% 88% 89% 90% 91% 92% 93% 94% 95% 96% 97% 98% or 99% identical to an amino acid sequence set forth in SEQ ID NOs: 2, 4, or 6.

The antibody or antigen binding fragment may have a heavy chain amino acid sequence with at least 80%, 85%, 90%, 91%, 92%, 93%, 94%, 95%, 96%, 97%, 98%, or 99% percent homology to the sequence as set forth in SEQ ID NO: 10, 9, or 7 and/or a light chain sequence with at least 80%, 85%, 90%, 91%, 92%, 93%, 94%, 95%, 96%, 97%, 98%, or 99% percent homology to the sequence as set forth in SEQ ID NO: 8.

The CDR sequences of the antibodies and binding fragments disclosed herein may be interposed between human or humanized framework sequences. The antibodies may be, for example, an intact or full length antibody.

The antibodies or antigen binding fragments disclosed herein may bind to at least one of the following residues: K550, R551, K585, H586, N587, G588, M589, W590, R591, L592, V593, G594, I595, T596, S597, Y617, M618, D619, W620, I621, L622, E623, K624, or T625 of human plasma kallikrein having the amino acid sequence set forth in SEQ ID NO:32. For example, they may bind to one or more of the following residues: K550, R551, K585, S597, Y617, or T625 of human plasma kallikrein having the amino acid sequence set forth in SEQ ID NO:32.

Antibodies or antigen binding fragments disclosed herein may bind to human plasma kallikrein, for example, human plasma kallikrein having the amino acid sequence set forth in SEQ ID NO:32, with a K_{D} of less than or equal to about 1, 2, 3, 4, or 5×10⁻⁹ M, as measured by surface plasmon resonance (SPR) assay. They may have a K_{D} that is substantially pH independent between pH 6.0-7.4 (e.g., as determined by SPR at 37° C. in a buffer containing 50 mM HEPES, 150 mM NaCl, 0.1 or 1.3 mM Ca²⁺, 1 mg/mL BSA, 0.02% Tween-20) and/or a K_{D} of between 0.1 nM and 5 nM for human plasma kallikrein (e.g., as determined by SPR at 37° C. in a buffer containing 50 mM HEPES, 150 mM NaCl, 0.1 or 1.3 mM Ca²⁺, 1 mg/mL BSA, 0.02% Tween-20, pH 6.0 or 7.4); and/or a K_{D} of between 0.1 nM and 5 nM for human plasma kallikrein and a K_{D} of between 250 nM and 2,000 nM for human prekallikrein (e.g., as determined by SPR at 37° C. in a buffer containing 50 mM HEPES, 150 mM NaCl, 0.1 or 1.3 mM Ca²⁺, 1 mg/mL BSA, 0.02% Tween-20, pH 6.0 or 7.4).

The invention is also directed to antibodies and binding fragments thereof that compete for binding to plasma kallikrein with the antibodies or binding fragments disclosed herein. For example, such antibodies may bind to the same epitope on human plasma kallikrein as the antibodies disclosed herein. For example, such antibodies may bind to one or more of residues K550, R551, K585, H586, N587, G588, M589, W590, R591, L592, V593, G594, I595, T596, S597, Y617, M618, D619, W620, I621, L622, E623, K624, or T625 of human plasma kallikrein having the amino acid sequence set forth in SEQ ID NO:32. For example, such antibodies may bind to one or more of residues K550, R551, K585, S597, Y617, or T625 of human plasma kallikrein having the amino acid sequence set forth in SEQ ID NO:32. Such antibodies may have one or more of the following features: (i) a serum half-life of at least 20 days; (ii) a K_{D} that is substantially pH independent between pH 6.0-7.4 (e.g., as determined by SPR at 37° C. in a buffer containing 50 mM HEPES, 150 mM NaCl, 0.1 or 1.3 mM Ca²⁺, 1 mg/mL BSA, 0.02% Tween-20); (iii) a K_{D} of between 0.1 nM and 5 nM for human plasma kallikrein (e.g., as determined by SPR at 37° C. in a buffer containing 50 mM HEPES, 150 mM NaCl, 0.1 or 1.3 mM Ca²⁺, 1 mg/mL BSA, 0.02% Tween-20, pH 6.0 or 7.4); and/or (iv) a K_{D} of between 0.1 nM and 5 nM for human plasma kallikrein and a K_{D} of between 250 nM and 2,000 nM for human prekallikrein (e.g., as determined by SPR at 37° C. in a buffer containing 50 mM HEPES, 150 mM NaCl, 0.1 or 1.3 mM Ca²⁺, 1 mg/mL BSA, 0.02% Tween-20, pH 6.0 or 7.4).

The antibodies or antigen binding fragments disclosed herein may be monoclonal antibodies. They may be humanized antibodies. They have may have a heavy chain constant region of a class selected from IgG, IgA, IgD, IgE, and IgM. If the constant region is of class IgG, the subclass may be selected from IgG1, IgG2, IgG3, and IgG4. If the heavy chain constant region is IgG1, it may have an M252Y, an S254T, and a T256E mutation or an M428L and a N434S mutation as numbered according to the EU numbering index. The heavy chain variable region and the light chain variable region may or may not be linked by a linker. If they are linked by a linker, such as in an scFv antibody, the linker may be a peptide linker having a sequence of (GGGGS)ₙ (SEQ ID NO:35); (GGGGA)ₙ (SEQ ID NO: 36), or any combination thereof, wherein each n is independently 1-5.

In another aspect, the invention includes isolated nucleic acids comprising a nucleotide sequence encoding the heavy chain or heavy chain variable region (VH) of the antibodies or binding fragments disclosed herein, as well as isolated nucleic acids comprising a nucleotide sequence encoding the light chain or light chain variable region (VL) of the antibody or antigen binding fragments disclosed herein. The heavy chain and light chain may be encoded on the same nucleic acid sequence, or the heavy chain may be encoded on one nucleic acid and the light chain encoded separately by another nucleic acid. The VL and VH may be encoded on the same nucleic acid sequence, or the VH may be encoded on one nucleic acid and the VL encoded separately by another nucleic acid. An expression vector may contain a nucleic acid encoding both the heavy and light chains, or an expression vector may contain a nucleic acid encoding both the VL and the VH chains. A host cell may contain an expression vector containing a nucleic acid encoding both the heavy and light chains, or a host cell may contain an expression vector containing a nucleic acid encoding the heavy chain and an expression vector containing a nucleic acid encoding the light chain. A host cell may contain an expression vector containing a nucleic acid encoding both the VH and VL, or a host cell may contain an expression vector containing a nucleic acid encoding the VH and an expression vector containing a nucleic acid encoding the VL. A host cell may be grown under conditions so that it expresses the heavy chain and light chain, or the VH and the VL to produce the antibody or the antigen-binding fragment of the invention.

In another aspect, the invention provides pharmaceutical compositions comprising the antibody or antigen binding fragments disclosed herein or the nucleic acids encoding the same. The pharmaceutical composition may, for example, be injectable, or administrable intravenously.

In yet another aspect, the invention provides methods of treating a subject with a disease or disorder selected from hereditary angioedema, bradykinin dependent edema, diabetic macular edema, retinal edema, Factor XII-associated cold autoinflammatory syndrome (FACAS), rheumatoid arthritis, gout, intestinal bowel disease, oral mucositis, neuropathic pain, inflammatory pain, spinal stenosis-degenerative spine disease, arterial or venous thrombosis, post-operative ileus, aortic aneurysm, osteoarthntis, vasculitis, edema, cerebral edema, pulmonary embolism, stroke, clotting induced by ventricular assistance devices or stents, head trauma or pen-tumor brain edema, sepsis, acute middle cerebral artery (MCA) ischemic event (stroke), restenosis (e.g., after angioplasty), systemic lupus erythematosus nephritis, or burn injury in a subject, by administering the plasma kallikrein antibodies or antigen binding fragment thereof as disclosed herein, or pharmaceutical composition containing the same.

The disclosed antibodies or antigen binding fragments may be used in a method of treating a plasma kallikrein-mediated disorder in a subject, including hereditary angioedema or bradykinin dependent edema. The disclosed antibodies or antigen binding fragments thereof may also be used in the manufacture of a medicament to treat the aforementioned conditions, such as hereditary angioedema or bradykinin dependent edema.

The disclosed antibodies or binding fragments thereof may also be used to reduce bradykinin levels in a subject, to reduce or inhibit bradykinin production in a subject, and/or to reduce or inhibit plasma kallikrein activity in a subject.

### BRIEF DESCRIPTION OF THE DRAWINGS

FIG. 1 is a bar graph showing the selectivity of various hybridoma lines for human plasma kallikrein based on relative light units (RLU).

FIG. 2 is a graph showing the inhibitory activity of various antibodies for human plasma kallikrein.

FIG. 3 provides graphs showing the inhibitory activity of various antibodies for human plasma kallikrein where the concentration of pKal is 1 nM (top graph) and 10 nM (top graph).

FIG. 4 provides graphs showing the inhibitory activity of various antibodies for human plasma kallikrein where the concentration of pKal is 1 nM.

FIG. 5 provides graphs showing that MAb4-YTE is more potent than DX-2930 in inhibiting Bradykinin production in vitro.

FIG. 6 provides graphs of surface plasmon resonance (SPR) binding data comparing the affinity of DX-2930, MAb4-YTE, and MAb4-LS for pKal (upper row of graphs) and preKal (lower row of graphs) at pH 7.4 and pH 6.0.

FIG. 7 is a graph showing plasma kallikrein (pKal) activity plotted against the concentration of MAb4-YTE in a cross-species pKal binding assay as described in Example 2. As it shows, MAb4-YTE is a potent inhibitor of not only human, but also monkey, rat, and rabbit pKal.

FIG. 8 provides a graph of pharmacokinetic data showing the concentration of MAb4, MAb4-LS, and MAb4-YTE over time in a cynomolgus monkey.

FIG. 9 provides graphs showing that MAb4-YTE has substantially prolonged plasma half-life compared to DX-2930 in vivo in cynomolgus monkeys. The concentration of the antibody over time for DX-2930 is shown in the top graph, for MAb4-YTE in the middle graph, and the data from the top and middle graph are shown together in the bottom graph.

FIG. 10 is a graph showing a longer duration of action for MAb4-YTE in vivo versus a comparative antibody.

FIG. 11 provides graphs showing the IC50 of gabexate mesylate, DX-2930, and MAb4-YTE in a trypsin inhibition assay.

FIG. 12 is a graph showing competitive binding of MAb4-LS and MAb4-YTE antibodies to a DX-2930 surface, competitive binding of MAb4-YTE and DX-2930 antibodies to a MAb4-LS surface, and competitive binding of MAb4-LS and DX-2930 antibodies to a MAb4-YTE surface.

FIG. 13 is a diagram showing the peptide interaction of human pKal and MAb4-YTE.

FIGS. 14A-J show the interaction between human pKal and MAb4-YTE. FIG. 14A shows a ribbon/surface representation of the front view of the pKal-MAb4-YTE epitope crosslink mapping. FIG. 14B shows a ribbon/surface representation of the back view of the pKal-MAb4-YTE epitope crosslink mapping. FIG. 14C shows a ribbon/surface representation of the side view 1 of the pKal-MAb4-YTE epitope crosslink mapping. FIG. 14D shows a ribbon/surface representation of the side view 2 of the pKal-MAb4-YTE epitope crosslink mapping. FIG. 14E shows a ribbon/surface representation of the top view of the pKal-MAb4-YTE epitope crosslink mapping. FIG. 14F shows a ribbon representation of the front view of the pKal-MAb4-YTE epitope crosslink mapping. FIG. 14G shows a ribbon representation of the back view of the pKal-MAb4-YTE epitope crosslink mapping. FIG. 14H shows a ribbon representation of the side view 1 of the pKal-MAb4-YTE epitope crosslink mapping. FIG. 14I shows a ribbon representation of the side view 2 of the pKal-MAb4-YTE epitope crosslink mapping. FIG. 14J shows a ribbon representation of the top view of the pKal-MAb4-YTE epitope crosslink mapping.

FIG. 15 is a diagram of the peptide interaction of human pKal and DX-2930.

FIGS. 16A-J show the interaction between human pKal and DX-2930. FIG. 16A shows a ribbon/surface representation of the front view of the pKal-DX-2930 epitope crosslink mapping. FIG. 16B shows a ribbon/surface representation of the back view of the pKal-DX-2930 epitope crosslink mapping. FIG. 16C shows a ribbon/surface representation of the side view 1 of the pKal-DX-2930 epitope crosslink mapping. FIG. 16D shows a ribbon/surface representation of the side view 2 of the pKal-DX-2930 epitope crosslink mapping. FIG. 16E shows a ribbons/surface representation of the top view of the pKal-DX-2930 epitope crosslink mapping. FIG. 16F shows a ribbon representation of the front view of the pKal-DX-2930 epitope crosslink mapping. FIG. 16G shows a ribbon representation of the back view of the pKal-DX-2930 epitope crosslink mapping. FIG. 16H shows a ribbon representation of the side view 1 of the pKal-DX-2930 epitope crosslink mapping. FIG. 16I shows a ribbon representation of the side view 2 of the pKal-DX-2930 epitope crosslink mapping. FIG. 16J shows a ribbon representation of the top view of the pKal-DX-2930 epitope crosslink mapping.

FIG. 17 is a map showing the residues of human pKal cross-linked by the antibody DX-2930 as compared to the antibody MAb4-YTE, as described in Example 5 below, and shows that these antibodies bind different epitopes on pKal.

FIGS. 18A-C show the nucleic acid sequence (top row; SEQ ID NO:48) and the amino acid sequence (bottom row; SEQ ID NO: 10) of the heavy chain of MAb4-YTE. The sequences start in FIG. 18A, continue into FIG. 18B and finish in FIG. 18C.

FIGS. 19A-B show the nucleic acid sequence (top row; SEQ ID NO: 49) and the amino acid sequence (bottom row; SEQ ID NO: 8) of the light chain of MAb4-YTE. The sequences start in FIG. 19A and finish in FIG. 19B.

### DETAILED DESCRIPTION OF THE INVENTION

The invention described herein is based, in part, upon the discovery of high affinity binding proteins, e.g., antibodies and antigen binding fragments, that bind to plasma kallikrein, e.g. human plasma kallikrein. The antibodies have high specificity for plasma kallikrein, e.g. human plasma kallikrein. The antibodies have high selectivity for plasma kallikrein relative to prekallikrein, and lack significant off-target binding to trypsin and other serine proteases. These binding proteins may also have extended half-lives, e.g., extended half-lives in blood. Because of these and other features, the antibodies may be useful for treating a variety of plasma kallikrein-mediated disorders, such as hereditary angioedema and bradykinin dependent edema, and also facilitating the treatment of these disorders with lower doses and/or extending dosing intervals as compared to other antibodies.

The antibodies described herein may have one or more of the following features: (i) a serum half-life of at least 20 days; (ii) a K_{D} that is substantially pH independent between pH 6.0-7.4 (e.g., as determined by SPR at 37° C. in a buffer containing 50 mM HEPES, 150 mM NaCl, 0.1 or 1.3 mM Ca²⁺, 1 mg/mL BSA, 0.02% Tween-20); (iii) a K_{D} of between 0.1 nM and 5 nM for human plasma kallikrein (e.g., as determined by SPR at 37° C. in a buffer containing 50 mM HEPES, 150 mM NaCl, 0.1 or 1.3 mM Ca²⁺, 1 mg/mL BSA, 0.02% Tween-20, pH 6.0 or 7.4); and/or (iv) a K_{D} of between 0.1 nM and 5 nM for human plasma kallikrein and a K_{D} of between 250 nM and 2,000 nM for human prekallikrein (e.g., as determined by SPR at 37° C. in a buffer containing 50 mM HEPES, 150 mM NaCl, 0.1 or 1.3 mM Ca²⁺, 1 mg/mL BSA, 0.02% Tween-20, pH 6.0 or 7.4).

These binding proteins can also be used in compositions, e.g., pharmaceutical compositions, for treating a variety of plasma kallikrein-mediated disorders such as hereditary angioedema and bradykinin dependent edema.

### I. Plasma Kallikrein Antibodies

As used herein, the term “antibody” refers to any form of antibody that exhibits the desired biological activity. Thus, the term “antibody” is used in the broadest sense and specifically covers, but is not limited to, monoclonal antibodies (including full length monoclonal antibodies), polyclonal antibodies, multi-specific antibodies (e.g., bispecific antibodies), humanized, fully human antibodies, chimeric antibodies and camelized single domain antibodies. The antibodies described herein include full length or intact antibodies which contain two immunoglobulin heavy chains associated with two immunoglobulin light chains.

An antibody light chain typically consists of one variable region (V_{L}) and one constant region (C_{L}). The heavy chain typically consists of one variable region (V_{H}) and at least three constant regions (CH₁, CH₂ and CH₃) (or more, depending on the isotype). The variable regions of each light/heavy chain pair, typically found at the amino-terminal portion of each chain, form the antibody binding site, which is primarily responsible for antigen recognition and determine the binding specificity of the antibody. Thus, in general, an intact or full length antibody has two binding sites, i.e., is bivalent. The two binding sites may be the same and target the same antigen; or, as in bifunctional or bispecific antibodies, the two binding sites may be different, e.g., each binding site targets a different antigen, or a different epitope of the same antigen.

The carboxy-terminal portion of the immunoglobulin heavy chains may define constant regions primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG (e.g., IgG1, IgG2, IgG3, IgG4), IgA, and IgE, respectively.

Typically, the variable domains of both the heavy and light chains of an antibody comprise three hypervariable regions, also called complementarity determining regions (CDRs), located within four relatively conserved framework regions (FRs). The CDRS are usually aligned by the framework regions, enabling binding to a specific epitope. In general, from N-terminal to C-terminal, both light and heavy chain variable domains comprise FR1, CDR1, FR2, CDR2, FR3, CDR3 and FR4. As used herein, “HCDR1,” HCDR2”, and “HCDR3” refer to CDR1, 2, or 3, respectively, of the heavy chain variable region, and “LCDR1,” LCDR2”, and “LCDR3” refer to CDR1, 2, or 3 respectively, of the light chain variable region.

The assignment of amino acids to each CDR is, generally, in accordance with the definitions of Kabat (See, e.g., Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem. 252:6609-6616) or the definitions of Chothia (See, e.g., Chothia, et al., (1987) J Mol. Biol. 196:901-917 or Chothia, et al., (1989) Nature 342:878-883), although alternative definitions are known in the art, such as IMGT (See e.g., Lefranc (2005), Nucl. Acids Res., 33, D593-D597; Lefranc et al., (2003), Dev. Comas Immunol., 27, 55-77; Lefranc et al., (2005), Dev. Comp. Immunol., 29, 185-20.3 (2005); Lefranc et al., (2005), Dev. Comp. Immunol., 2005, 29, 917.938) or AbM (See, e.g., Martin, et al., (1989) Proc. Natl. Acad. Sci. USA, 4, 86, 9268-9272; Pedersen et al., (1992), Immunomethods, 1, 126; Rees et al., (1996) In Sternberg M. J. E. (ed.). Protein Structure Prediction. Oxford University Press, Oxford, 141-172).

As used herein, the term “parental antibodies” is used to refer to antibodies obtained by exposure of an immune system to an antigen prior to modification of the antibodies for an intended use, such as humanization of an antibody for use as a human therapeutic antibody.

As used herein, unless otherwise indicated, the term “antibody fragment” or “antigen binding fragment” refers to antigen binding fragments of antibodies, i.e. antibody fragments that retain the ability to bind specifically to the antigen bound by the full-length or intact antibody, e.g. fragments that retain one or more CDR regions. Examples of antibody binding fragments include, but are not limited to, Fab, Fab′, F(ab′)₂, and Fv fragments; diabodies; linear antibodies; single-chain antibody molecules, e.g., sc-Fv; nanobodies and multi-specific antibodies formed from antibody fragments; single-domain antibodies; recombinant heavy-chain-only antibodies (VHH); and shark heavy-chain-only antibodies (VNAR).

As used herein, the term “Fab fragment” is understood to mean an antibody fragment comprising one light chain and the C_{H}1 and variable regions of one heavy chain.

As used herein, the term “Fc” region refers to an antibody fragment containing two heavy chain fragments each comprising at least the C_{H}2 and C_{H}3 domain of an antibody.

As used herein, the term “Fab′ fragment” is understood to mean an antibody fragment that contains one light chain and a portion or fragment of one heavy chain that contains the V_{H} domain and the C_{H}1 domain and also the region between the C_{H}1 and C_{H}2 domains. An interchain disulfide bond formed between the two heavy chains of two Fab′ fragments can form an F(ab′)₂ fragment.

The term “Fv region” as used herein refers to a region of an antibody that comprises the variable regions from both the heavy and light chains, but lacks the constant regions.

The terms “single-chain Fv” or “scFv” refer to antibody fragments comprising the V_{H} and V_{L} domains of an antibody, wherein these domains are present in a single polypeptide chain. Typically, an scFv polypeptide includes a polypeptide linker between the V_{H} and V_{L} domains which enables the scFv to form the desired structure for antigen binding. For a review of scFv, see, e.g., Pluckthun (1994) THE PHARMACOLOGY OF MONOCLONAL ANTIBODIES, vol. 113, Rosenburg and Moore eds. Springer-Verlag, New York, pp. 269-315. See also, International Patent Application Publication No. WO 88/01649 and U.S. Pat. Nos. 4,946,778 and 5,260,203.

The term “monoclonal antibody”, as used herein, refers to population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, that are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

As used herein, the term “chimeric antibody” is understood to mean an antibody having the variable domain from a first antibody and constant domain from a second antibody, where the first and second antibodies are from different species, see, e.g., U.S. Pat. No. 4,816,567; and Morrison et al., (1984) Proc. Nat. Acad. Sci. USA 81: 6851-6855).

As used herein, the term “humanized antibody” refers to forms of antibodies that contain sequences from both human and non-human (e.g., murine, rat) antibodies. For example, a humanized antibody can comprise variable regions where the CDRs or portions thereof are derived from or correspond to sequences of a non-human immunoglobulin and all or substantially all of the FRs or portions thereof are derived from or correspond to sequences of human immunoglobulin. The humanized antibody may optionally comprise at least a portion of a human immunoglobulin constant region (e.g., human light chain constant region and/or human heavy chain constant region, e.g., a human Fc, or human CH1, CH2, and/or CH3 domains). The CDR sequences described herein may be interposed between human or humanized framework sequences.

Humanization can be undertaken to reduce the immunogenicity of an antibody, e.g., a non-human antibody, when administered to a human. In one humanization approach, chimeric proteins are created in which mouse immunoglobulin constant regions are replaced with human immunoglobulin constant regions. See, e.g., Morrison et al., 1984, PROC. NAT. ACAD. SCI. 81:6851-6855, Neuberger et al., 1984, NATURE 312:604-608; U.S. Pat. No. 6,893,625 (Robinson); U.S. Pat. No. 5,500,362 (Robinson); and U.S. Pat. No. 4,816,567 (Cabilly).

In an approach known as CDR grafting, the CDRs of the light and heavy chain variable regions are grafted into frameworks from another species. For example, murine CDRs can be grafted into human FRs. In some embodiments, the CDRs of the light and heavy chain variable regions of an antibody are grafted into human FRs or consensus human FRs. To create consensus human FRs, FRs from several human heavy chain or light chain amino acid sequences are aligned to identify a consensus amino acid sequence. CDR grafting is described in U.S. Pat. No. 7,022,500 (Queen); U.S. Pat. No. 6,982,321 (Winter); U.S. Pat. No. 6,180,370 (Queen); U.S. Pat. No. 6,054,297 (Carter); U.S. Pat. No. 5,693,762 (Queen); U.S. Pat. No. 5,859,205 (Adair); U.S. Pat. No. 5,693,761 (Queen); U.S. Pat. No. 5,565,332 (Hoogenboom); U.S. Pat. No. 5,585,089 (Queen); U.S. Pat. No. 5,530,101 (Queen); Jones et al. (1986) NATURE 321: 522-525; Riechmann et al. (1988) NATURE 332: 323-327; Verhoeyen et al. (1988) S_{CIENCE} 239: 1534-1536; and Winter (1998) FEBs LETT 430: 92-94.

In an approach called “SUPERHUMANIZATION™,” human CDR sequences are chosen from human germline genes, based on the structural similarity of the human CDRs to those of the mouse antibody to be humanized. See, e.g., U.S. Pat. No. 6,881,557 (Foote); and Tan et al., 2002, J. IMMUNOL. 169:1119-1125.

Other methods to reduce immunogenicity include “reshaping,” “hyperchimerization,” and “veneering/resurfacing.” See, e.g., Vaswami et al., 1998, ANNALS OF ALLERGY, ASTHMA, & IMMUNOL. 81:105; Roguska et al., 1996, PROT. ENGINEER 9:895-904; and U.S. Pat. No. 6,072,035 (Hardman). In the veneering/resurfacing approach, the surface accessible amino acid residues in the murine antibody are replaced by amino acid residues more frequently found at the same positions in a human antibody. This type of antibody resurfacing is described, e.g., in U.S. Pat. No. 5,639,641 (Pedersen).

Another approach for converting a mouse antibody into a form suitable for medical use in humans is known as ACTIVMAB™ technology (Vaccinex, Inc., Rochester, NY), which involves a vaccinia virus-based vector to express antibodies in mammalian cells. High levels of combinatorial diversity of IgG heavy and light chains can be produced. See, e.g., U.S. Pat. No. 6,706,477 (Zauderer); U.S. Pat. No. 6,800,442 (Zauderer); and U.S. Pat. No. 6,872,518 (Zauderer). Another approach for converting a mouse antibody into a form suitable for use in humans is technology practiced commercially by KaloBios Pharmaceuticals, Inc. (Palo Alto, CA). This technology involves the use of a proprietary human “acceptor” library to produce an “epitope focused” library for antibody selection. Another approach for modifying a mouse antibody into a form suitable for medical use in humans is HUMAN ENGINEERING™ technology, which is practiced commercially by XOMA (US) LLC. See, e.g., International (PCT) Publication No. WO 93/11794 and U.S. Pat. No. 5,766,886 (Studnicka); U.S. Pat. No. 5,770,196 (Studnicka); U.S. Pat. No. 5,821,123 (Studnicka); and U.S. Pat. No. 5,869,619 (Studnicka).

The antibodies disclosed herein can also be converted to different types, such as being converted to human IgGs and the like. By converting the antibodies to a human antibody, a human subject should not identify the antibodies as foreign. The conversion of a non-human IgG antibody to a human IgG antibody is well known and can routinely be done once the native sequence is known. As discussed herein, the antibodies can be modified according to known methods. Such methods are described in, for example, Riechmann L, Clark M, Waldmann H, Winter G (1988). Reshaping human antibodies for therapy”. Nature 332 (6162): 332-323; Tsurushita N, Park M, Pakabunto K, Ong K, Avdalovic A, Fu H, Jia A, Vasquez M, Kumar S. (2004), which is incorporated by reference in its entirety. Accordingly, antibodies of the invention may be humanized antibodies.

As used herein, the term “fully human antibody” refers to an antibody that comprises human immunoglobulin protein sequences only. A fully human antibody may contain murine carbohydrate chains if produced in a mouse, in a mouse cell, or in a hybridoma derived from a mouse cell.

As used herein, the terms “specific binding,” “immunospecific binding,” “binds immunospecifically,” or “binds specifically” refer to antibody binding to a predetermined antigen (e.g. plasma kallikrein) or epitope present on the antigen. In some embodiments, the antibody binds a predetermined antigen (e.g. plasma kallikrein) with a dissociation constant (K_{D}) of 10⁻⁷ M or less and/or binds to the predetermined antigen with a K_{D} that is at least two-fold less than its K_{D} for binding to a non-specific antigen (e.g., BSA, casein, or another non-specific polypeptide) other than the predetermined antigen. The phrases “an antibody recognizing plasma kallikrein” and “an antibody specific for plasma kallikrein” are used interchangeably herein with the term “an antibody which binds immunospecifically to plasma kallikrein.” In some embodiments, the antibody binds specifically or preferentially to plasma kallikrein over other proteins, such as, but not limited to human prekallikrein. In some embodiments, the antibody, or antigen binding fragment of the antibody, binds to its antigen (plasma kallikrein) with an affinity that is at least two fold greater, at least ten times greater, at least 20-times greater, or at least 100-times greater than the affinity with any other antigen.

Methods for determining mAb specificity and affinity by competitive inhibition can be found in Harlow, et al., Antibodies: A Laboratory Manual, Cold Spring Harbor Laboratory Press, Cold Spring Harbor, N.Y., 1988), Colligan et al., eds., Current Protocols in Immunology, Greene Publishing Assoc. and Wiley Interscience, N.Y., (1992, 1993), and Muller, Meth. Enzymol. 92:589 601 (1983), which references are entirely incorporated herein by reference.

As used herein, the term “homolog” refers to a protein sequence having at least 40% but less than 100% sequence homology or identity to a reference sequence. Percent identity between two peptide chains can be determined by pair wise alignment using the default settings of the AlignX module of Vector NTI v.9.0.0 (Invitrogen Corp., Carlsbad, Calif.) or other suitable alignment software, such as BLAST. In some embodiments, the antibody, or antigenic binding fragment thereof has, at least 50, 60, 70, 80, 90, 91, 92, 93, 94, 95, 96, 97, 98, or 99% homology or identity to a sequence described herein. In some embodiments, the antibody has conservative substitutions as compared to a sequence described herein. Exemplary conservative substitutions are illustrated in Table 1 and are encompassed within the scope of the disclosed subject matter. The conservative substitution may reside in the framework regions, or in antigen-binding sites, as long they do not adversely affect the properties of the antibody. Substitutions may be made to improve antibody properties, for example stability or affinity. Conservative substitutions will produce molecules having functional and chemical characteristics similar to those molecules into which such modifications are made. Exemplary amino acid substitutions are shown in Table 1 below.

TABLE 1
Exemplary Conservative Substitutions:

| Original | Exemplary Conservative |
|---|---|
| Residue | Substitutions |
| Ala | Val, Leu, Ile |
| Arg | Lys, Gln, Asn |
| Asn | Gln |
| Asp | Glu |
| Cys | Ser, Ala |
| Gln | Asn |
| Gly | Pro, Ala |
| His | Asn, Gln, Lys, Arg |
| Ile | Leu, Val, Met, Ala, Phe |
| Leu | Ile, Val, Met, Ala, Phe |
| Lys | Arg, Gln, Asn |
| Met | Leu, Phe, Ile |
| Phe | Leu, Val, Ile, Ala, Tyr |
| Pro | Ala |
| Ser | Thr, Ala, Cys |
| Thr | Ser |
| Trp | Tyr, Phe |
| Tyr | Trp, Phe, Thr, Ser |
| Val | Ile, Met, Leu, Phe, Ala |

In some embodiments, variants of the proteins and peptides provided herein are provided. In some embodiments, a variant comprises a substitution, deletion, or insertion. In some embodiments, the variant comprises 1, 2, 3, 4, 5, 6, 7, 8, 9, or 10 (e.g., 1-10) substitutions. As described herein, the substitutions can be conservative substitutions. In some embodiments, the substitution is non-conservative. In some embodiments, the variant comprises 1, 2, 3, 4, 5, 6, 7, 8, 9, or 10 (e.g., 1-10) deletions. In some embodiments, the variant comprises 1, 2, 3, 4, 5, 6, 7, 8, 9, or 10 (e.g., 1-10) insertions. In some embodiments, the substitutions, deletions, or insertions are present in the CDRs provided for herein. In some embodiments, the substitutions, deletions, or insertions are not present in the CDRs provided for herein.

Typically, a variant antibody or antigen binding fragment of the antibodies provided herein retains at least 10% of its plasma kallikrein binding activity (when compared to the unmodified or reference antibody, which can be a parental antibody) when that activity is expressed on a molar basis. In some embodiments, a variant antibody (or antigen fragment thereof), or antigen binding fragment of an antibody provided herein, retains at least 20%, 50%, 70% 80% 90% 91% 92% 93% 94% 95% 96% 97% 98% 99% or 100% or more of the plasma kallikrein binding affinity as the unmodified or reference antibody, which can be a parental antibody. As described herein, it is also intended that an antibody or antigen binding fragment of the invention can include conservative or non-conservative amino acid substitutions, which can also be referred to as “conservative variants” or “function conserved variants” of the antibody, that do not substantially alter its biologic activity.

As used herein, the term “epitope” refers to a portion of any molecule capable of being recognized by and bound by an antibody at one or more of the Ab's antigen binding regions. Epitopes usually consist of chemically active surface groupings of molecules such as amino acids or sugar side chains and have specific three dimensional structural characteristics as well as specific charge characteristics. Example of epitopes include, but are not limited to, the residues that the antibodies provided for herein bind to. An epitope may be a linear or sequential epitope, that is, a linear sequence of amino acids of the primary structure of the antigen, for example, human plasma kallikrein (SEQ ID NO: 32). Alternatively, in other embodiments, an epitope may be a conformational epitope having a specific three-dimensional shape when the antigen assumes its secondary structure. For example, the conformational epitope may comprise non-linear, i.e., non-sequential, amino acids of the antigen.

The antibodies disclosed herein may be inhibitory antibodies e.g., with high potency, specificity, and prolonged serum residency, where the high potency translates to increased efficacy with a low drug dosage, and high specificity reduces side effects due to the inhibition of related off-target senile proteases.

Antibodies or an antigen binding fragments thereof that bind to a plasma kallikrein, wherein the antibody or antigen binding fragment thereof comprises an amino acid sequence, or a variant thereof, as provided for herein, may comprise one or more of the following features: a serum half-life of at least 20 days; a K_{D} that is substantially pH independent between pH 6.0-7.4 (e.g., as determined by SPR at 37° C. in a buffer containing 50 mM HEPES, 150 mM NaCl, 0.1 or 1.3 mM Ca2+, 1 mg/mL BSA, 0.02% Tween-20); a K_{D} of between 0.1 nM and 5 nM for human plasma kallikrein (e.g., as determined by SPR at 37° C. in a buffer containing 50 mM HEPES, 150 mM NaCl, 0.1 or 1.3 mM Ca2+, 1 mg/mL BSA, 0.02% Tween-20, pH 6.0 or 7.4); and/or a KD of between 0.1 nM and 5 nM for human plasma kallikrein and a K_{D} of between 250 nM and 2,000 nM for human prekallikrein (e.g., as determined by SPR at 37° C. in a buffer containing 50 mM HEPES, 150 mM NaCl, 0.1 or 1.3 mM Ca2+, 1 mg/mL BSA, 0.02% Tween-20, pH 6.0 or 7.4).

In some embodiments, the antibody is a monoclonal antibody which binds to plasma kallikrein. In some embodiments, the antibody binds to amino acids of an epitope of the plasma kallikrein. In some embodiments, the plasma kallikrein is human plasma kallikrein (UNIPROT P03952, KLKB1) which has the following amino acid sequence:

(SEQ ID NO: 32)
MILFKQATYF ISLFATVSCG CLTQLYENAF FRGGDVASMY
TPNAQYCQMR CTFHPRCLLF SFLPASSIND MEKRFGCFLK
DSVTGTLPKV HRTGAVSGHS LKQCGHQISA CHRDIYKGVD
MRGVNFNVSK VSSVEECQKR CINNIRCQFF SYATQTFHKA
EYRNNCLLKY SPGGTPTAIK VLSNVESGFS LKPCALSEIG
CHMNIFQHLA FSDVDVARVL TPDAFVCRTI CTYHPNCLFF
TFYTNVWKIE SQRNVCLLKT SESGTPSSST PQENTISGYS
LLTCKRTLPE PCHSKIYPGV DFGGEELNVT FVKGVNVCQE
TCTKMIRCQF FTYSLLPEDC KEEKCKCFLR LSMDGSPTRI
AYGTQGSSGY SLRLCNTGDN SVCTTKTSTR IVGGTNSSWG
EWPWQVSLQV KLTAQRHLCG GSLIGHQWVL TAAHCFDGLP
LQDVWRIYSG ILNLSDITKD TPFSQIKEII IHQNYKVSEG
NHDIALIKLQ APLNYTEFQK PICLPSKGDT STIYTNCWVT
GWGFSKEKGE IQNILQKVNI PLVTNEECQK RYQDYKITQR
MVCAGYKEGG KDACKGDSGG PLVCKHNGMW RLVGITSWGE
GCARREQPGV YTKVAEYMDW ILEKTQSSDG KAQMQSPA

In some embodiments, the plasma kallikrein is cynomolgus monkey plasma kallikrein (UNIPROT A0A2K5VTJ9-1, SEQ ID NO: 45), rabbit plasma kallikrein (UNIPROT GIT127, SEQ ID NO: 46), or rat plasma kallikrein (UNIPROT P14272, SEQ ID NO: 47). For example, the antibodies or antigen binding fragments thereof may bind to rabbit, rat, or monkey plasma kallikrein.

The sequence of human plasma kallikrein as illustrated above includes a signal peptide sequence of amino acid residues 1-19, which can be cleaved during its post-translation processing. In some embodiments, the antibody binds to human plasma kallikrein without the signal peptide sequence, e.g., mature human plasma kallikrein.

In some embodiments, the antibody comprises an amino acid sequence as provided for herein.

The sequences of the antibodies can be modified to yield human IgG antibodies. The conversion of the sequences provided herein can be modified to yield other types of antibodies. The CDRs can also be linked to other antibodies, proteins, or molecules to create antibody fragments that bind to plasma kallikrein. This can be in the form of an antibody drug conjugate (“ADC”), or a multi-specific molecule. The sequences can also be made into chimeric antibodies as described herein.

In some embodiments, the antibody comprises an amino acid sequence comprising a sequence provided for herein or a fragment thereof. In some embodiments, the antibody comprises one or more amino acid sequences as provided herein, an antigen binding fragments, thereof, or a human IgG variant thereof. “A human IgG variant thereof” refers to an antibody that has been modified to be a human IgG when the starting antibody is not a human IgG antibody.

The antibodies can also be modified to be chimeric antibodies or human antibodies. The antibodies can also be used in injectable pharmaceutical compositions. As also described herein, the antibodies can be isolated antibodies or engineered antibodies.

In some embodiments, “derivatives” of the antibodies, fragments, regions or derivatives thereof, which term includes those proteins encoded by truncated or modified genes to yield molecular species functionally resembling the immunoglobulin fragments are provided. The modifications include, but are not limited to, addition of genetic sequences coding for cytotoxic proteins such as plant and bacterial toxins. The modification can also include a reporter protein, such as a fluorescent or chemiluminescent tag. The fragments and derivatives can be produced in any manner.

The identification of these antigen binding region and/or epitopes recognized by Abs described herein provide the information necessary to generate additional monoclonal antibodies with similar binding characteristics and therapeutic or diagnostic utility that parallel the embodiments of this application.

The variable regions described herein can be combined with any type of constant region including a human constant region or murine constant region. Human genes which encode the constant (C) regions of the antibodies, fragments and regions can be derived from a human fetal liver library, by known methods. Human C regions genes can be derived from any human cell including those which express and produce human immunoglobulins. The human C_{H} region can be derived from any of the known classes or isotypes of human H chains, including gamma, μ, α, δ or ε, and subtypes thereof, such as G1, G2, G3 and G4. Since the H chain isotype is responsible for the various effector functions of an antibody, the choice of C_{H} region will be guided by the desired effector functions, such as complement fixation, or activity in antibody-dependent cellular cytotoxicity (ADCC). Preferably, the C_{H} region is derived from gamma 1 (IgG1), gamma 3 (IgG3), gamma 4 (IgG4), or μ (IgM). The human C_{L} region can be derived from either human L chain isotype, kappa or lambda.

In some embodiments, the antibody comprises a Fc domain. In some embodiments, the Fc domain comprises a mutation to extend the half-life of the antibody. In some embodiments, the Fc domain comprises a mutation such as those described in U.S. Pat. No. 7,670,600, which is hereby incorporated by reference in its entirety. In some embodiment, the constant region comprises a mutation at position at amino acid residue 428 relative to a wild-type human IgG constant domain, numbered according to the EU numbering index of Kabat. Without being bound to any particular theory, an antibody comprising a mutation that corresponds to residue 428 can have an increased half-life compared to the half-life of an IgG having the wild-type human IgG constant domain. In some embodiments, the mutation is a substitution of the native residue with a threonine, leucine, phenylalanine or serine. In some embodiments, the antibody further comprises one or more amino acid substitutions relative to the corresponding wild-type human IgG constant domain at one or more of amino acid residues 251-256, 285-290, 308-314, 385-389, and 429-436, numbered according to the Kabat EU numbering index. The specific mutations or substitutions at these positions are described in U.S. Pat. No. 7,670,600, which is hereby incorporated by reference in its entirety.

Other mutations can be used in the Fc domain, such as those provided for in U.S. Pat. No. 8,394,925, which is hereby incorporated by reference in its entirety. In some embodiments, the Fc region is a variant Fc region comprising amino acid substitutions at positions 428 and 434, wherein the amino acid substitutions are a leucine that is not the wild-type amino acid at position 428 and a serine that is not the wild-type amino acid at position 434, wherein the polypeptide is an antibody and wherein numbering is according to the EU Index in Kabat et al. In some embodiments, the Fc region comprises a S228P, L235E, M428L, or N434S substitution. In some embodiments, the Fc region comprises a M428L substitution. In some embodiments, the Fc region comprises a N434S substitution. In some embodiments, the Fc region comprises a M428L and a N434S substitution wherein numbering is according to the EU Index in Kabat et al. In some embodiments, the Fc region comprises a M252Y, S254T, and/or T256E substitution or the Fc region comprises a M252Y, S254T, and T256E substitution, wherein numbering is according to the EU Index in Kabat et al.

In some embodiments, the antibodies described herein are used to detect the presence of the antigen. The present antibody can be used in any device or method to detect the presence of the antigen.

### I.A. Antibody CDR Sequences

An antibody, or antigen binding fragment, disclosed herein may be defined by its CDR sequences. For example, the antibody or antigen binding fragment may include one or more of the amino acid sequences provided in Table 2, which are CDRs of the antibody or antigen binding fragment as defined by Kabat numbering.

TABLE 2
CDRs Defined by Kabat

| Antibody | | | | | | |
|---|---|---|---|---|---|---|
| Name | LCDR1 | LCDR2 | LCDR3 | HCDR1 | HCDR2 | HCDR3 |
| MAb1 | KASQDVGI | WASTRHT | QQYRSYPL | SYWMN | QIYPGDDDTNY | SLMVTTGA |
| | AVA (SEQ | (SEQ ID | T (SEQ ID | (SEQ ID | NGKFKG (SEQ | PFDY (SEQ |
| | ID NO: 11) | NO: 12) | NO: 13) | NO: 14) | ID NO: 15) | ID NO: 16) |
| MAb2 | KASQDVGI | WASTRHT | QQYRSYPL | SYWMN | QIYPGDDDTNY | SLMVTTGA |
| | AVA (SEQ | (SEQ ID | T (SEQ ID | (SEQ ID | NGKFQG (SEQ | PFDY (SEQ |
| | ID NO: 11) | NO: 12) | NO: 13) | NO: 14) | ID NO: 17) | ID NO: 16) |
| MAb | KASQDVGI | YASHRGW | QQYRSYPL | SYWMN | QIYPGDDDTNY | SLMVTTGA |
| 3/4 | AVA (SEQ | (SEQ ID | T (SEQ ID | (SEQ ID | NAKFQG (SEQ | PFDY (SEQ |
| | ID NO: 11) | NO: 18) | NO: 13) | NO: 14) | ID NO: 19) | ID NO: 16) |

The antibody or antigen binding fragment may include one or more of the amino acid sequences provided in Table 3, which are CDRs of the antibody or antigen binding fragment as defined by Chothia numbering.

TABLE 3
CDRs defined by Chothia

| Antibody | | | | | | |
|---|---|---|---|---|---|---|
| Name | LCDR1 | LCDR2 | LCDR3 | HCDR1 | HCDR2 | HCDR3 |
| MAb1 | SQDVGIA | WAS | YRSYPLT | GYAFSSY | YPGDDD | SLMVTTGAPFD |
| | (SEQ ID | (SEQ ID | (SEQ ID | (SEQ ID | (SEQ ID | Y |
| | NO: 20) | NO: 21) | NO: 22) | NO: 23) | NO: 24) | (SEQ ID NO: 25) |
| MAb2 | SQDVGIA | WAS | YRSYPLT | GYAFSSY | YPGDDD | SLMVTTGAPFD |
| | (SEQ ID | (SEQ ID | (SEQ ID | (SEQ ID NO: | (SEQ ID | Y |
| | NO: 20) | NO: 21) | NO: 22) | 23) | NO: 24) | (SEQ ID NO: 25) |
| MAb | SQDVGIA | YAS | YRSYPLT | GYAFSSY | YPGDDD | SLMVTTGAPFD |
| 3/4 | (SEQ ID | (SEQ ID | (SEQ ID | (SEQ ID | (SEQ ID | Y |
| | NO: 20) | NO: 26) | NO: 22) | NO: 23) | NO: 24) | (SEQ ID NO: 25) |

The antibody or antigen binding fragment may include one or more of the amino acid sequences provided in Table 4, which are CDRs of the antibody or antigen binding fragment as defined by IMGT numbering.

TABLE 4
CDRs defined by IMGT

| Antibody | | | | | | |
|---|---|---|---|---|---|---|
| Name | LCDR1 | LCDR2 | LCDR3 | HCDR1 | HCDR2 | HCDR3 |
| MAbl | QDVGIA | WAS | QQYRSYPL | GYAFSSYW | IYPGDDDT | AGSLMVTTGAP |
| | (SEQ ID | (SEQ ID | T | (SEQ ID | (SEQ ID | FDY |
| | NO: 27) | NO: 21) | (SEQ ID | NO: 29) | NO: 30) | (SEQ ID NO: 31) |
| | | | NO: 28) | | | |
| MAb2 | QDVGIA | WAS | QQYRSYPL | GYAFSSYW | IYPGDDDT | AGSLMVTTGAP |
| | (SEQ ID | (SEQ ID | T | (SEQ ID | (SEQ ID | FDY |
| | NO: 27) | NO: 21) | (SEQ ID | NO: 29) | NO: 30) | (SEQ ID NO: 31) |
| | | | NO: 28) | | | |
| MAb 3/4 | QDVGIA | YAS | QQYRSYPL | GYAFSSYW | IYPGDDDT | AGSLMVTTGAP |
| | (SEQ ID | (SEQ ID | T | (SEQ ID | (SEQ ID | FDY |
| | NO: 27) | NO: 26) | (SEQ ID | NO: 29) | NO: 30) | (SEQ ID NO: 31) |
| | | | NO: 28) | | | |

The antibody or antigen binding fragment may include one or more of the amino acid sequences provide in Table 5, which are CDRs of the antibody or antigen binding fragment as defined by AbM numbering.

TABLE 5
CDRs defined by ABM

| Antibody | | | | | | |
|---|---|---|---|---|---|---|
| Name | LCDR1 | LCDR2 | LCDR3 | HCDR1 | HCDR2 | HCDR3 |
| MAb1 | KASQDVG | WASTRH | QQYRSYPL | GYAFSSYW | QIYPGDDDT | SLMVTTGAP |
| | IAVA (SEQ | T (SEQ ID | T (SEQ ID | MN (SEQ ID | N (SEQ ID | FDY (SEQ ID |
| | ID NO: 11) | NO: 12) | NO: 28) | NO: 43) | NO: 44) | NO: 25) |
| MAb2 | KASQDVG | WASTRH | QQYRSYPL | GYAFSSYW | QIYPGDDDT | SLMVTTGAP |
| | IAVA | T (SEQ ID | T (SEQ ID | MN (SEQ ID | N (SEQ ID | FDY (SEQ ID |
| | (SEQ ID | NO: 12) | NO: 28) | NO: 43) | NO: 44) | NO: 25) |
| | NO: 11) | | | | | |
| MAb 3/4 | KASQDVG | YASHRG | QQYRSYPL | GYAFSSYW | QIYPGDDDT | SLMVTTGAP |
| | IAVA (SEQ | W (SEQ ID | T (SEQ ID | MN (SEQ ID | N (SEQ ID | FDY (SEQ ID |
| | ID NO: 11) | NO: 18) | NO: 28) | NO: 43) | NO: 44) | NO: 25) |

An antibody, or antigen binding fragment thereof, may include a heavy or light chain CDR having a sequence of SEQ ID NOs: 11, 12, 13, 14, 15, 16, 17, 18, 19, 20, 21, 22, 23, 24, 25 26, 27, 28, 29, 30, or 31. For example, a light chain CDR of the antibody, or antigen binding fragment thereof may have the sequence of SEQ ID NO: 11, 12, 13, 18, 20, 21, 22, 26, 27, or 28. For example, a heavy chain CDR of the antibody, or antigen binding fragment thereof may have the sequence of SEQ ID NO: 14, 15, 16, 17, 19, 23, 24, 25, 29, 30, or 31.xx

The CDRs described herein can be interchanged with the CDRs that are characterized by different definitions, such as Chothia and IMGT, which are illustrated in the tables above. For example, the LCDR1 defined by Kabat can be interchanged with the LCDR1 defined by Chothia, IMGT or AbM; the LCDR2 defined by Kabat can be interchanged with the LCDR2 defined by Chothia, IMGT, or AbM, and so on. For example, in some embodiments, an antibody, or antigen binding fragment thereof, comprises a light chain variable region having an LCDR1, an LCDR2, and an LCDR3, wherein the LCDR1 has the sequence of SEQ ID NO: 11, 20, or 27; the LCDR2 has the sequence of SEQ ID NO: 12, 18, 21, or 26; and the LCDR3 has the sequence of SEQ ID NO: 13, 22 or 28; while in some embodiments, the antibody or antigen binding fragment thereof comprises a heavy chain variable region having an HCDR1, an HCDR2, and an HCDR3, wherein the HCDR1 has the sequence of SEQ ID NO: 14, 23, 29, or 43; the HCDR2 has the sequence of SEQ ID NO: 15, 17, 19, 24, 30, or 44; and the HCDR3 has the sequence of SEQ ID NO: 16, 25, or 31.

In some embodiments, an antibody, or antigen binding fragment thereof, comprises a light chain variable region having an LCDR1, an LCDR2, and an LCDR3, wherein the LCDR1 has the sequence of SEQ ID NO: 11, the LCDR2 has the sequence of SEQ ID NO: 12, and the LCDR3 has the sequence of SEQ ID NO: 13.

In some embodiments, an antibody, or antigen binding fragment thereof, comprises a light chain variable region having an LCDR1, an LCDR2, and an LCDR3, wherein the LCDR1 has the sequence of SEQ ID NO: 11, the LCDR2 has the sequence of SEQ ID NO: 18, and the LCDR3 has the sequence of SEQ ID NO: 13.

In some embodiments, an antibody, or antigen binding fragment thereof, comprises a light chain variable region having an LCDR1, an LCDR2, and an LCDR3, wherein the LCDR1 has the sequence of SEQ ID NO: 20, the LCDR2 has the sequence of SEQ ID NO: 21, and the LCDR3 has the sequence of SEQ ID NO: 22.

In some embodiments, an antibody, or antigen binding fragment thereof, comprises a light chain variable region having an LCDR1, an LCDR2, and an LCDR3, wherein the LCDR1 has the sequence of SEQ ID NO: 20, the LCDR2 has the sequence of SEQ ID NO: 26, and the LCDR3 has the sequence of SEQ ID NO: 22.

In some embodiments, an antibody, or antigen binding fragment thereof, comprises a light chain variable region having an LCDR1, an LCDR2, and an LCDR3, wherein the LCDR1 has the sequence of SEQ ID NO: 27, the LCDR2 has the sequence of SEQ ID NO: 21, and the LCDR3 has the sequence of SEQ ID NO: 28.

In some embodiments, an antibody, or antigen binding fragment thereof, comprises a light chain variable region having an LCDR1, an LCDR2, and an LCDR3, wherein the LCDR1 has the sequence of SEQ ID NO: 27, the LCDR2 has the sequence of SEQ ID NO: 26, and the LCDR3 has the sequence of SEQ ID NO: 28.

In some embodiments, an antibody, or antigen binding fragment thereof, comprises a light chain variable region having an LCDR1, an LCDR2, and an LCDR3, wherein the LCDR1 has the sequence of SEQ ID NO: 11, the LCDR2 has the sequence of SEQ ID NO: 12, and the LCDR3 has the sequence of SEQ ID NO:28.

In some embodiments, an antibody, or antigen binding fragment thereof, comprises a light chain variable region having an LCDR1, an LCDR2, and an LCDR3, wherein the LCDR1 has the sequence of SEQ ID NO: 11, the LCDR2 has the sequence of SEQ ID NO: 18, and the LCDR3 has the sequence of SEQ ID NO: 28.

In some embodiments, an antibody, or antigen binding fragment thereof, comprises a heavy chain variable region having an HCDR1, an HCDR2, and an HCDR3, wherein the HCDR1 has the sequence of SEQ ID NO: 14, the HCDR2 has the sequence of SEQ ID NO: 15 and the HCDR3 has the sequence of SEQ ID NO: 16.

In some embodiments, an antibody, or antigen binding fragment thereof, comprises a heavy chain variable region having an HCDR1, an HCDR2, and an HCDR3, wherein the HCDR1 has the sequence of SEQ ID NO: 14, the HCDR2 has the sequence of SEQ ID NO: 17 and the HCDR3 has the sequence of SEQ ID NO: 16.

In some embodiments, an antibody, or antigen binding fragment thereof, comprises a heavy chain variable region having an HCDR1, an HCDR2, and an HCDR3, wherein the HCDR1 has the sequence of SEQ ID NO: 14, the HCDR2 has the sequence of SEQ ID NO: 19 and the HCDR3 has the sequence of SEQ ID NO: 16.

In some embodiments, an antibody, or antigen binding fragment thereof, comprises a heavy chain variable region having an HCDR1, an HCDR2, and an HCDR3, wherein the HCDR1 has the sequence of SEQ ID NO: 23, the HCDR2 has the sequence of SEQ ID NO: 24 and the HCDR3 has the sequence of SEQ ID NO: 25.

In some embodiments, an antibody, or antigen binding fragment thereof, comprises a heavy chain variable region having an HCDR1, an HCDR2, and an HCDR3, wherein the HCDR1 has the sequence of SEQ ID NO: 29, the HCDR2 has the sequence of SEQ ID NO: 30 and the HCDR3 has the sequence of SEQ ID NO: 31.

In some embodiments, an antibody, or antigen binding fragment thereof, comprises a heavy chain variable region having an HCDR1, an HCDR2, and an HCDR3, wherein the HCDR1 has the sequence of SEQ ID NO: 43, the HCDR2 has the sequence of SEQ ID NO: 44 and the HCDR3 has the sequence of SEQ ID NO: 25.

The different CDR motifs can be combined in any combination including those not depicted in the tables above. For example, the following embodiments are provided as non-limiting examples of such combinations.

In some embodiments, an antibody, or antigen binding fragment thereof, comprises: (i) a light chain variable region comprising an LCDR1, an LCDR2, and an LCDR3, wherein the LCDR1 has the amino acid sequence of SEQ ID NO: 11; the LCDR2 has the amino acid sequence of SEQ ID NO: 12 or 18; and the LCDR3 sequence has the amino acid sequence of SEQ ID NO: 13 and (ii) a heavy chain variable region comprising an HCDR1, an HCDR2, and an HCDR3, wherein the HCDR1 sequence has the amino acid sequence of SEQ ID NO: 14; the heavy chain CDR2 sequence has the amino acid sequence of SEQ ID NO: 15, 17, or 19; and the heavy chain CDR3 sequence has the amino acid sequence of SEQ ID NO: 16; or variants of any of the foregoing.

In some embodiments, an antibody, or antigen binding fragment thereof, comprises: (i) a light chain variable region comprising an LCDR1, an LCDR2, and an LCDR3, wherein the LCDR1 has the amino acid sequence of SEQ ID NO: 11; the LCDR2 has the amino acid sequence of SEQ ID NO: 12; and the LCDR3 sequence has the amino acid sequence of SEQ ID NO: 13 and (ii) a heavy chain variable region comprising an HCDR1, an HCDR2, and an HCDR3, wherein the HCDR1 sequence has the amino acid sequence of SEQ ID NO: 14; the heavy chain CDR2 sequence has the amino acid sequence of SEQ ID NO: 15; and the heavy chain CDR3 sequence has the amino acid sequence of SEQ ID NO: 16; or variants of any of the foregoing.

In some embodiments, an antibody, or antigen binding fragment thereof, comprises: (i) a light chain variable region comprising an LCDR1, an LCDR2, and an LCDR3, wherein the LCDR1 has the amino acid sequence of SEQ ID NO: 11; the LCDR2 has the amino acid sequence of SEQ ID NO: 12; and the LCDR3 sequence has the amino acid sequence of SEQ ID NO: 13 and (ii) a heavy chain variable region comprising an HCDR1, an HCDR2, and an HCDR3, wherein the HCDR1 sequence has the amino acid sequence of SEQ ID NO: 14; the heavy chain CDR2 sequence has the amino acid sequence of SEQ ID NO: 17; and the heavy chain CDR3 sequence has the amino acid sequence of SEQ ID NO: 16; or variants of any of the foregoing.

In some embodiments, an antibody, or antigen binding fragment thereof, comprises: (i) a light chain variable region comprising an LCDR1, an LCDR2, and an LCDR3, wherein the LCDR1 has the amino acid sequence of SEQ ID NO: 11; the LCDR2 has the amino acid sequence of SEQ ID NO: 12; and the LCDR3 sequence has the amino acid sequence of SEQ ID NO: 13 and (ii) a heavy chain variable region comprising an HCDR1, an HCDR2, and an HCDR3, wherein the HCDR1 sequence has the amino acid sequence of SEQ ID NO: 14; the heavy chain CDR2 sequence has the amino acid sequence of SEQ ID NO: 19; and the heavy chain CDR3 sequence has the amino acid sequence of SEQ ID NO: 16; or variants of any of the foregoing.

In some embodiments, an antibody, or antigen binding fragment thereof, comprises: (i) a light chain variable region comprising an LCDR1, an LCDR2, and an LCDR3, wherein the LCDR1 has the amino acid sequence of SEQ ID NO: 11; the LCDR2 has the amino acid sequence of SEQ ID NO: 18; and the LCDR3 sequence has the amino acid sequence of SEQ ID NO: 13 and (ii) a heavy chain variable region comprising an HCDR1, an HCDR2, and an HCDR3, wherein the HCDR1 sequence has the amino acid sequence of SEQ ID NO: 14; the heavy chain CDR2 sequence has the amino acid sequence of SEQ ID NO: 15; and the heavy chain CDR3 sequence has the amino acid sequence of SEQ ID NO: 16; or variants of any of the foregoing.

In some embodiments, an antibody, or antigen binding fragment thereof, comprises: (i) a light chain variable region comprising an LCDR1, an LCDR2, and an LCDR3, wherein the LCDR1 has the amino acid sequence of SEQ ID NO: 11; the LCDR2 has the amino acid sequence of SEQ ID NO: 18; and the LCDR3 sequence has the amino acid sequence of SEQ ID NO: 13 and (ii) a heavy chain variable region comprising an HCDR1, an HCDR2, and an HCDR3, wherein the HCDR1 sequence has the amino acid sequence of SEQ ID NO: 14; the heavy chain CDR2 sequence has the amino acid sequence of SEQ ID NO: 17; and the heavy chain CDR3 sequence has the amino acid sequence of SEQ ID NO: 16; or variants of any of the foregoing.

In some embodiments, an antibody, or antigen binding fragment thereof, comprises: (i) a light chain variable region comprising an LCDR1, an LCDR2, and an LCDR3, wherein the LCDR1 has the amino acid sequence of SEQ ID NO: 11; the LCDR2 has the amino acid sequence of SEQ ID NO: 18; and the LCDR3 sequence has the amino acid sequence of SEQ ID NO: 13 and (ii) a heavy chain variable region comprising an HCDR1, an HCDR2, and an HCDR3, wherein the HCDR1 sequence has the amino acid sequence of SEQ ID NO: 14; the heavy chain CDR2 sequence has the amino acid sequence of SEQ ID NO: 19; and the heavy chain CDR3 sequence has the amino acid sequence of SEQ ID NO: 16; or variants of any of the foregoing.

In some embodiments, an antibody, or antigen binding fragment thereof, comprises: (i) a light chain variable region comprising an LCDR1, an LCDR2, and an LCDR3, wherein the LCDR1 has the amino acid sequence of SEQ ID NO: 20; the LCDR2 has the amino acid sequence of SEQ ID NO: 21 or 26; and the LCDR3 sequence has the amino acid sequence of SEQ ID NO: 22 and (ii) a heavy chain variable region comprising an HCDR1, an HCDR2, and an HCDR3, wherein the HCDR1 sequence has the amino acid sequence of SEQ ID NO: 23; the heavy chain CDR2 sequence has the amino acid sequence of SEQ ID NO: 24; and the heavy chain CDR3 sequence has the amino acid sequence of SEQ ID NO: 25; or variants of any of the foregoing.

In some embodiments, an antibody, or antigen binding fragment thereof, comprises: (i) a light chain variable region comprising an LCDR1, an LCDR2, and an LCDR3, wherein the LCDR1 has the amino acid sequence of SEQ ID NO: 20; the LCDR2 has the amino acid sequence of SEQ ID NO: 21; and the LCDR3 sequence has the amino acid sequence of SEQ ID NO: 22 and (ii) a heavy chain variable region comprising an HCDR1, an HCDR2, and an HCDR3, wherein the HCDR1 sequence has the amino acid sequence of SEQ ID NO: 23; the heavy chain CDR2 sequence has the amino acid sequence of SEQ ID NO: 24; and the heavy chain CDR3 sequence has the amino acid sequence of SEQ ID NO: 25; or variants of any of the foregoing.

In some embodiments, an antibody, or antigen binding fragment thereof, comprises: (i) a light chain variable region comprising an LCDR1, an LCDR2, and an LCDR3, wherein the LCDR1 has the amino acid sequence of SEQ ID NO: 20; the LCDR2 has the amino acid sequence of SEQ ID NO: 26; and the LCDR3 sequence has the amino acid sequence of SEQ ID NO: 22 and (ii) a heavy chain variable region comprising an HCDR1, an HCDR2, and an HCDR3, wherein the HCDR1 sequence has the amino acid sequence of SEQ ID NO: 23; the heavy chain CDR2 sequence has the amino acid sequence of SEQ ID NO: 24; and the heavy chain CDR3 sequence has the amino acid sequence of SEQ ID NO: 25; or variants of any of the foregoing.

In some embodiments, an antibody, or antigen binding fragment thereof, comprises: (i) a light chain variable region comprising an LCDR1, an LCDR2, and an LCDR3, wherein the LCDR1 has the amino acid sequence of SEQ ID NO: 27; the LCDR2 has the amino acid sequence of SEQ ID NO: 21 or 26; and the LCDR3 sequence has the amino acid sequence of SEQ ID NO: 28 and (ii) a heavy chain variable region comprising an HCDR1, an HCDR2, and an HCDR3, wherein the HCDR1 sequence has the amino acid sequence of SEQ ID NO: 29; the heavy chain CDR2 sequence has the amino acid sequence of SEQ ID NO: 30; and the heavy chain CDR3 sequence has the amino acid sequence of SEQ ID NO: 31; or variants of any of the foregoing.

In some embodiments, an antibody, or antigen binding fragment thereof, comprises: (i) a light chain variable region comprising an LCDR1, an LCDR2, and an LCDR3, wherein the LCDR1 has the amino acid sequence of SEQ ID NO: 27; the LCDR2 has the amino acid sequence of SEQ ID NO: 21; and the LCDR3 sequence has the amino acid sequence of SEQ ID NO: 28 and (ii) a heavy chain variable region comprising an HCDR1, an HCDR2, and an HCDR3, wherein the HCDR1 sequence has the amino acid sequence of SEQ ID NO: 29; the heavy chain CDR2 sequence has the amino acid sequence of SEQ ID NO: 30; and the heavy chain CDR3 sequence has the amino acid sequence of SEQ ID NO: 31; or variants of any of the foregoing.

In some embodiments, an antibody, or antigen binding fragment thereof, comprises: (i) a light chain variable region comprising an LCDR1, an LCDR2, and an LCDR3, wherein the LCDR1 has the amino acid sequence of SEQ ID NO: 27; the LCDR2 has the amino acid sequence of SEQ ID NO: 26; and the LCDR3 sequence has the amino acid sequence of SEQ ID NO: 28 and (ii) a heavy chain variable region comprising an HCDR1, an HCDR2, and an HCDR3, wherein the HCDR1 sequence has the amino acid sequence of SEQ ID NO: 29; the heavy chain CDR2 sequence has the amino acid sequence of SEQ ID NO: 30; and the heavy chain CDR3 sequence has the amino acid sequence of SEQ ID NO: 31; or variants of any of the foregoing.

In some embodiments, an antibody, or antigen binding fragment thereof, comprises: (i) a light chain variable region comprising an LCDR1, an LCDR2, and an LCDR3, wherein the LCDR1 has the amino acid sequence of SEQ ID NO: 11; the LCDR2 has the amino acid sequence of SEQ ID NO: 12 or 18; and the LCDR3 sequence has the amino acid sequence of SEQ ID NO: 28 and (ii) a heavy chain variable region comprising an HCDR1, an HCDR2, and an HCDR3, wherein the HCDR1 sequence has the amino acid sequence of SEQ ID NO: 43; the heavy chain CDR2 sequence has the amino acid sequence of SEQ ID NO: 44; and the heavy chain CDR3 sequence has the amino acid sequence of SEQ ID NO: 25; or variants of any of the foregoing.

In some embodiments, an antibody, or antigen binding fragment thereof, comprises: (i) a light chain variable region comprising an LCDR1, an LCDR2, and an LCDR3, wherein the LCDR1 has the amino acid sequence of SEQ ID NO: 11; the LCDR2 has the amino acid sequence of SEQ ID NO: 12; and the LCDR3 sequence has the amino acid sequence of SEQ ID NO: 28 and (ii) a heavy chain variable region comprising an HCDR1, an HCDR2, and an HCDR3, wherein the HCDR1 sequence has the amino acid sequence of SEQ ID NO: 43; the heavy chain CDR2 sequence has the amino acid sequence of SEQ ID NO: 44; and the heavy chain CDR3 sequence has the amino acid sequence of SEQ ID NO: 25; or variants of any of the foregoing.

In some embodiments, an antibody, or antigen binding fragment thereof, comprises: (i) a light chain variable region comprising an LCDR1, an LCDR2, and an LCDR3, wherein the LCDR1 has the amino acid sequence of SEQ ID NO: 11; the LCDR2 has the amino acid sequence of SEQ ID NO: 18; and the LCDR3 sequence has the amino acid sequence of SEQ ID NO: 28 and (ii) a heavy chain variable region comprising an HCDR1, an HCDR2, and an HCDR3, wherein the HCDR1 sequence has the amino acid sequence of SEQ ID NO: 43; the heavy chain CDR2 sequence has the amino acid sequence of SEQ ID NO: 44; and the heavy chain CDR3 sequence has the amino acid sequence of SEQ ID NO: 25; or variants of any of the foregoing.

In some embodiments, the HCDR1 comprises the amino acid sequence set forth in SEQ ID NO: 14 with up to 1, 2, 3, or 4 amino acid substitutions to the amino acid sequence; the HCDR2 comprises the amino acid sequence set forth in SEQ ID NO: 15, 17, or 19 with up to 1, 2, 3, 4, 5, 6, 7, 8, 9, 10, 11, 12, or 13 amino acid substitutions to the amino acid sequence; and the HCDR3 comprises the amino acid sequence set forth in SEQ ID NO: 16 with up to 1, 2, 3, 4, 5, 6, 7, 8, or 9 amino acid substitutions to the amino acid sequence. In some embodiments, the LCDR1 comprises the amino acid sequence set forth in SEQ ID NO: 11 with up to 1, 2, 3, 4, 5, 6, or 7 amino acid substitutions to the amino acid sequence; the LCDR2 comprises the amino acid sequence set forth in SEQ ID NO: 12 or 18 with up to 1, 2, 3, or 4 amino acid substitutions to the amino acid sequence; and the LCDR3 comprises the amino acid sequence set forth in SEQ ID NO: 13 with up to 1, 2, 3, or 4 amino acid substitutions to the amino acid sequence. The amino acid substitutions may be conservative amino acid substitutions. In some embodiments, the antibodies described in this paragraph are referred to herein as “variants.” These variants may be derived from a sequence provided herein, for example, by affinity maturation, site directed mutagenesis, random mutagenesis, or any other method known in the art or described herein. Alternatively, these variants are not derived from a sequence provided herein and may, for example, be isolated de novo according to the methods provided herein for obtaining antibodies.

In some embodiments, the HCDR1 comprises the amino acid sequence set forth in SEQ ID NO: 14, 23, 29 or 43 with up to 1, 2, 3, 4, 5, 6, 7, or 8 amino acid substitutions to the amino acid sequence; the HCDR2 comprises the amino acid sequence set forth in SEQ ID NO: 15, 17, 19, 24, 30, or 44 with up to 1, 2, 3, 4, 5, 6, 7, 8, 9, 10, 11, 12, or 13 amino acid substitutions to the amino acid sequence; and the HCDR3 comprises the amino acid sequence set forth in SEQ ID NO: 16, 25, or 31 with up to 1, 2, 3, 4, 5, 6, 7, 8, or 9 amino acid substitutions to the amino acid sequence. In some embodiments, the LCDR1 comprises the amino acid sequence set forth in SEQ ID NO: 11, 20, or 27 with up to 1, 2, 3, 4, 5, 6, or 7 amino acid substitutions to the amino acid sequence; the LCDR2 comprises the amino acid sequence set forth in SEQ ID NO: 12, 18, 21, or 26 with up to 1, 2, 3, or 4 amino acid substitutions to the amino acid sequence; and the LCDR3 comprises the amino acid sequence set forth in SEQ ID NO: 13, 22, or 28, with up to 1, 2, 3, 4, 5, or 6 amino acid substitutions to the amino acid sequence. The amino acid substitutions may be conservative amino acid substitutions. In some embodiments, the antibodies described in this paragraph are referred to herein as “variants.” These variants may be derived from a sequence provided herein, for example, by affinity maturation, site directed mutagenesis, random mutagenesis, or any other method known in the art or described herein. Alternatively, these variants are not derived from a sequence provided herein and may, for example, be isolated de novo according to the methods provided herein for obtaining antibodies.

In some embodiments, an antibody, or antigen binding fragment thereof, comprises: (i) a light chain variable region having an LCDR1, an LCDR2, and an LCDR3, wherein the LCDR1 has a sequence selected from SEQ ID NO: 11, 20, or 27 wherein the LCDR1 comprises at most one conservative amino acid substitution, the LCDR2 has a sequence selected from SEQ ID NO: 12, 18, 21, and 26 wherein the LCDR2 comprises at most one conservative amino acid substitution, and the LCDR3 has a sequence selected from SEQ ID NO: 13, 22, and 28 wherein the LCDR3 comprises at most one conservative amino acid substitution and (ii) a heavy chain variable region comprising an HCDR1, an HCDR2, and an HCDR3, wherein the HCDR1 has a sequence selected from SEQ ID NO: 14, 23, or 29 wherein the HCDR1 comprises at most one conservative amino acid substitution, the HCDR2 has a sequence selected from SEQ ID NO: 15, 19, 24, or 30 wherein the HCDR2 comprises at most one conservative amino acid substitution, and the HCDR3 has a sequence selected from SEQ ID NO: 16, 25, or 31 wherein the HCDR3 comprises at most one conservative amino acid substitution.

In some embodiments, an antibody, or antigen binding fragment thereof, comprises: (i) a light chain variable region having an LCDR1, an LCDR2, and an LCDR3, wherein the LCDR1 has the sequence of SEQ ID NO: 11 wherein the LCDR1 comprises at most one conservative amino acid substitution, the LCDR2 has the sequence of SEQ ID NO: 18 wherein the LCDR2 comprises at most one conservative amino acid substitution, and the LCDR3 has the sequence selected from SEQ ID NO: 13 wherein the LCDR3 comprises at most one conservative amino acid substitution and (ii) a heavy chain variable region comprising an HCDR1, an HCDR2, and an HCDR3, wherein the HCDR1 has the sequence of SEQ ID NO: 14 wherein the HCDR1 comprises at most one conservative amino acid substitution, the HCDR2 has the sequence of SEQ ID NO: 19 wherein the HCDR2 comprises at most one conservative amino acid substitution, and the HCDR3 has the sequence of SEQ ID NO: 16 wherein the HCDR3 comprises at most one conservative amino acid substitution.

In some embodiments, the light chain variable region CDR1 is replaced with any of the other light chain CDR1 sequences disclosed herein. In some embodiments, the light chain variable region CDR2 is replaced with any of the other light chain CDR2 sequences disclosed herein. In some embodiments, the light chain variable region CDR3 is replaced with any of the other light chain CDR3 sequences disclosed herein. In some embodiments, the heavy chain variable region CDR1 is replaced with any of the other heavy chain CDR1 sequences disclosed herein. In some embodiments, the heavy chain variable region CDR2 is replaced with any of the other heavy chain CDR2 sequences disclosed herein. In some embodiments, the heavy chain variable region CDR3 is replaced with any of the other heavy chain CDR3 sequences disclosed herein.

The antibodies of the invention or binding fragments thereof may have an HCDR1, an HCDR2, and/or an HCDR3 with a sequence corresponding to the sequence of the HCDR1, HCDR2 and/or HCDR3 of the heavy chain variable region sequence of SEQ ID NO: 1, 3, or 5; and/or an LCDR1, an LCDR2 and/or an LCDR3 with a sequence corresponding to the sequence of the LCDR1, LCDR2 and/or LCDR3 of the light chain variable region sequence of SEQ ID NO:2, 4, or 6. For example, the antibodies of the invention or binding fragments thereof may have an HCDR1, an HCDR2, and an HCDR3 with a sequence corresponding to the sequence of the HCDR1, HCDR2 and HCDR3 of the heavy chain variable region sequence of SEQ ID NO:3 or 5; and an LCDR1, an LCDR2, and an LCDR3 with a sequence corresponding to the sequence of the LCDR1, the LCDR2, and the LCDR3 of the light chain variable region sequence of SEQ ID NO:4 or 6. For example, the antibodies of the invention or binding fragments thereof may have an HCDR1, an HCDR2, and an HCDR3 with a sequence corresponding to the sequence of the HCDR1, HCDR2 and HCDR3 of the heavy chain variable region sequence of SEQ ID NO:5; and an LCDR1, an LCDR2, and an LCDR3 with a sequence corresponding to the sequence of the LCDR1, the LCDR2, and the LCDR3 of the light chain variable region sequence of SEQ ID NO:6. The CDRs may be defined by Kabat, by Chothia, by IMGT, or by AbM. For example, the CDRs are as defined by Kabat. For example, the CDRs are as defined by Chothia.

### I.B. Antibody Variable and Full Length Sequences

In some embodiments, the antibody comprises one or more peptides having the following sequences, or a variant thereof as provided in Table 6:

TABLE 6

| SEQ ID | | |
|---|---|---|
| NO: | AB ID NO. | Sequence |
| 1 | MAb1 heavy | QVQLQQSGAELVKPGASVKISCKASGYAFSSYWMNWVKQRPGEGL |
| | chain variable | EWIGQIYPGDDDTNYNGKFKGKATLTVDKSSTTAYMQLSSLTSED |
| | region (VH) | SAVYFCAGSLMVTTGAPFDYWGQGTTLTVSS |
| 2 | MAb1 light | DIVMTQSHKFMSTSVGDRVSITCKASQDVGIAVAWYQQKPGQSPK |
| | chain variable | FLIYWASTRHTGVPDRFTGSGSGTDFTLTIRNVQSEDLSDYFCQQ |
| | region (VL) | YRSYPLTFGSGTKLELK |
| 3 | MAb2 VH | QVQLVQSGAEVKKPGSSVKVSCKASGYAFSSYWMNWVRQAPGQGL |
| | | EWIGQIYPGDDDTNYNGKFQGRVTITVDKSTTTAYMELSSLRSED |
| | | TAVYFCAGSLMVTTGAPFDYWGQGTTVTVSS |
| 4 | MAb2 VL | DIQMTQSPSSLSASVGDRVTITCKASQDVGIAVAWYQQKPGKAPK |
| | | FLIYWASTRHTGVPDRFSGSGSGTDFTLTISSLQPEDFATYFCQQ |
| | | YRSYPLTFGQGTKLEIK |
| 5 | MAb3-VH | QVQLVQSGAEVKKPGSSVKVSCKASGYAFSSYWMNWVRQAPGQGL |
| | MAb4-VH | EWIGQIYPGDDDTNYNAKFQGRVTITVDKSTTTAYMELSSLRSED |
| | | TAVYFCAGSLMVTTGAPFDYWGQGTTVTVSS |
| 6 | MAb3-VL | DIQMTQSPSSLSASVGDRVTITCKASQDVGIAVAWYQQKPGKAPK |
| | MAb4-VL | FLIYYASHRGWGVPDRFSGSGSGTDFTLTISSLQPEDFATYFCQQ |
| | | YRSYPLTFGQGTKLEIK |
| 7 | MAb4 full | QVQLVQSGAEVKKPGSSVKVSCKASGYAFSSYWMNWVRQAPGQGL |
| | length heavy | EWIGQIYPGDDDTNYNAKFQGRVTITVDKSTTTAYMELSSLRSED |
| | chain (HC) | TAVYFCAGSLMVTTGAPFDYWGQGTTVTVSSASTKGPSVFPLAPS |
| | IgG1 format | SKSTSGGTAALGCLVKDYFPEPVTVSWNSGALTSGVHTFPAVLQS |
| | | SGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDV |
| | | SHEDPEVKFNWYVDGVEVHNAKTKPREEQYNSTYRVVSVLTVLHQ |
| | | DWLNGKEYKCKVSNKALPAPIEKTISKAKGQPREPQVYTLPPSRE |
| | | EMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPG |
| 8 | MAb4 full | DIQMTQSPSSLSASVGDRVTITCKASQDVGIAVAWYQQKPGKAPK |
| | length light | FLIYYASHRGWGVPDRFSGSGSGTDFTLTISSLQPEDFATYFCQQ |
| | chain (LC) | YRSYPLTFGQGTKLEIKRTVAAPSVFIFPPSDEQLKSGTASVVCL |
| | human kappa | LNNFYPREAKVQWKVDNALQSGNSQESVTEQDSKDSTYSLSSTLT |
| | format | LSKADYEKHKVYACEVTHQGLSSPVTKSENRGEC |
| 9 | MAb4 heavy | QVQLVQSGAEVKKPGSSVKVSCKASGYAFSSYWMNWVRQAPGQGL |
| | chain (HC) | EWIGQIYPGDDDTNYNAKFQGRVTITVDKSTTTAYMELSSLRSED |
| | IgG1 format | TAVYFCAGSLMVTTGAPFDYWGQGTTVTVSSASTKGPSVFPLAPS |
| | with M428L | SKSTSGGTAALGCLVKDYFPEPVTVSWNSGALTSGVHTFPAVLQS |
| | and N434S | SGLYSLSSVVTVPSSSLGTQTYICNVNHKPSNTKVDKRVEPKSCD |
| | half-life | KTHTCPPCPAPELLGGPSVFLFPPKPKDTLMISRTPEVTCVVVDV |
| | extension | SHEDPEVKFNWYVDGVEVHNAKTKPREEQYNSTYRVVSVLTVLHQ |
| | substitutions | DWLNGKEYKCKVSNKALPAPIEKTISKAKGQPREPQVYTLPPSRE |
| | (underlined) | EMTKNQVSLTCLVKGFYPSDIAVEWESNGQPENNYKTTPPVLDSD |
| | | GSFFLYSKLTVDKSRWQQGNVFSCSVLHEALHSHYTQKSLSLSPG |
| 10 | MAb4 heavy | QVQLVQSGAEVKKPGSSVKVSCKASGYAFSSYWMNWVRQAPGQGL |
| | chain (HC) | EWIGQIYPGDDDTNYNAKFQGRVTITVDKSTTTAYMELSSLRSED |
| | IgG1 format | TAVYFCAGSLMVTTGAPFDYWGQGTTVTVSSASTKGPSVFPLAPS |
| | with M252Y, | SKSTSGGTAALGCLVKDYFPEPVTVSWNSGALTSGVHTFPAVLQS |
| | S254T, and | SGLYSLSSVVTVPSSSLGTQTYICNVNHKPSNTKVDKRVEPKSCD |
| | T256E half- | KTHTCPPCPAPELLGGPSVFLFPPKPKDTLYITREPEVTCVVVDV |
| | life extension | SHEDPEVKFNWYVDGVEVHNAKTKPREEQYNSTYRVVSVLTVLHQ |
| | substitutions | DWLNGKEYKCKVSNKALPAPIEKTISKAKGQPREPQVYTLPPSRE |
| | (underlined) | EMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPG |

In some embodiments, the antibody or antigen binding fragment comprises a sequence selected from one or more of the following sequences: SEQ ID NO: 1; SEQ ID NO: 2; SEQ ID NO: 3; SEQ ID NO: 4; SEQ ID NO: 5, SEQ ID NO: 6, SEQ ID NO: 7, SEQ ID NO: 8, SEQ ID NO: 9, SEQ ID NO: 10, or a variant thereof.

In some embodiments, the antibody or antigen binding fragment comprises a heavy chain variable region comprising the amino acid sequence of SEQ ID NO:1, 3, or 5, and a light chain variable region comprising the amino acid sequence of SEQ ID NO: 2, 4, or 6.

In some embodiments, the antibody or antigen binding fragment comprises a heavy chain variable region comprising the amino acid sequence of SEQ ID NO: 1, and a light chain variable region comprising the amino acid sequence of SEQ ID NO: 2, 4, or 6.

In some embodiments, the antibody or antigen binding fragment comprises a heavy chain variable region comprising the amino acid sequence of SEQ ID NO:3, and a light chain variable region comprising the amino acid sequence of SEQ ID NO: 2, 4, or 6.

In some embodiments, the antibody or antigen binding fragment comprises a heavy chain variable region comprising the amino acid sequence of SEQ ID NO:5, and a light chain variable region comprising the amino acid sequence of SEQ ID NO: 2, 4, or 6.

In some embodiments, the antibody or antigen binding fragment comprises a heavy chain variable region comprising the amino acid sequence of SEQ ID NO: 1 and a light chain variable region comprising the amino acid sequence of SEQ ID NO: 2, or the antibody comprises a heavy chain comprising the amino acid sequence of SEQ ID NO: 1, and a light chain comprising the amino acid sequence of SEQ ID NO:2.

In some embodiments, the antibody or antigen binding fragment comprises a heavy chain variable region comprising the amino acid sequence of SEQ ID NO: 3 and a light chain variable region comprising the amino acid sequence of SEQ ID NO: 4, or the antibody comprises a heavy chain comprising the amino acid sequence of SEQ ID NO: 3, and a light chain comprising the amino acid sequence of SEQ ID NO:4.

In some embodiments, the antibody or antigen binding fragment comprises a heavy chain variable region comprising the amino acid sequence of SEQ ID NO: 5 and a light chain variable region comprising the amino acid sequence of SEQ ID NO: 6. In some embodiments, the antibody comprises a heavy chain comprising the amino acid sequence of SEQ ID NO: 5, and a light chain comprising the amino acid sequence of SEQ ID NO:6.

In some embodiments, the antibody or antigen binding fragment comprises a heavy chain variable region comprising the amino acid sequence of SEQ ID NO: 5 or the antibody comprises a heavy chain comprising the amino acid sequence of SEQ ID NO: 5.

In some embodiments, the antibody or antigen binding fragment comprises a light chain variable region comprising the amino acid sequence of SEQ ID NO: 6, or the antibody comprises a light chain comprising the amino acid sequence of SEQ ID NO:6.

In some embodiments, the antibody comprises a heavy chain comprising the amino acid sequence of SEQ ID NO: 1, 3, 5, 7, 9, or 10 and a light chain comprising the amino acid sequence of SEQ ID NO: 2, 4, 6, or 8.

In some embodiments, the antibody comprises a heavy chain comprising the amino acid sequence of SEQ ID NO: 7, 9, or 10, and a light chain comprising the amino acid sequence of SEQ ID NO: 8.

In some embodiments, the antibody comprises a heavy chain comprising the amino acid sequence of SEQ ID NO: 7 and a light chain comprising the amino acid sequence of SEQ ID NO: 8.

In some embodiments, the antibody comprises a heavy chain comprising the amino acid sequence of SEQ ID NO: 9 and a light chain comprising the amino acid sequence of SEQ ID NO: 8.

In some embodiments, the antibody comprises a heavy chain comprising the amino acid sequence of SEQ ID NO: 10 and a light chain comprising the amino acid sequence of SEQ ID NO: 8.

In some embodiments, the antibody or antigen binding fragment comprises a heavy chain variable region comprising an amino acid sequence that is at least 70%, 80%, 85%, 86%, 87% 88% 89% 90% 91% 92% 93% 94% 95% 96% 97% 98% or 99% identical to the amino acid sequence set forth in SEQ ID NOs: 1, 3, or 5.

An antibody or antigen binding fragment provided herein may comprise a heavy chain variable region comprising an amino acid sequence provided in SEQ ID NOs: 1, 3, or 5, with up to 1, 2, 3, 4, 5, 6, 7, 8, 9, 10, 11, 12, 13, 14, 15, 16, 17, 18, 19, 20, 21, 22, 23, 24, or 25 amino acid substitutions. For example, one or more substitutions may be found solely in the CDR regions, solely in the framework regions, or in both the CDR and framework regions. For example, the heavy chain variable region may have at least 70%, 80%, 85%, 86%, 87%, 88%, 89%, 90%, 91%, 92%, 93%, 94%, 95%, 96%, 97%, 98%, or 99% identity with the amino acid sequence of SEQ ID NO: 1, 3, or 5, wherein any variation from SEQ ID NO: 1, 3, or 5 does not occur within HCDR1, HCDR2, or HCDR3. For example, the variation is found in one or more framework regions. In some examples, the amino acid substitution(s) may be (a) conservative amino acid substitution(s). The antibodies described in this paragraph are referred to herein as “variants.” Such variants may be derived from a sequence provided herein, for example, by affinity maturation, site directed mutagenesis, random mutagenesis, or any other method known in the art or described herein. Alternatively, such variants may not be derived from a sequence provided herein and may, for example, be isolated de novo according to the methods provided herein for obtaining antibodies.

In some embodiments, an antibody or antigen binding fragment comprises a light chain variable region comprising an amino acid sequence that is at least 70%, 80%, 85%, 86%, 87%, 88% 89% 90% 91% 92% 93% 94% 95% 96% 97% 98% or 99% identical to the amino acid sequence set forth in SEQ ID NOs: 2, 4, or 6.

An antibody or antigen binding fragment provided herein may comprise a light chain variable region comprising an amino acid sequence provided in SEQ ID NOs: 2, 4, or 6, with up to 1, 2, 3, 4, 5, 6, 7, 8, 9, 10, 11, 12, 13, 14, 15, 16, 17, 18, 19, 20, 21, 22, 23, 24, or 25 amino acid substitutions. For example, one or more substitutions may be found solely in the CDR regions, solely in the framework regions, or in both the CDR and framework regions. For example, the light chain variable region may have at least 70%, 80%, 85%, 86%, 87%, 88%, 89%, 90%, 91%, 92%, 93%, 94%, 95%, 96%, 97%, 98%, or 99% identity with SEQ ID NO: 2, 4, or 6, wherein any variation from SEQ ID NO: 2, 4, or 6 does not occur within LCDR1, LCDR2, or LCDR3. For example, the variation is found in one or more framework regions. In some examples, the amino acid substitution(s) may be (a) conservative amino acid substitution(s). The antibodies described in this paragraph are referred to herein as “variants.” Such variants may be derived from a sequence provided herein, for example, by affinity maturation, site directed mutagenesis, random mutagenesis, or any other method known in the art or described herein. Alternatively, such variants may not be derived from a sequence provided herein and may, for example, be isolated de novo according to the methods provided herein for obtaining antibodies.

The heavy chain variable region (VH) and the light chain variable region (VL) sequences can be in any format, including, but not limited to an scFv format where the VH and VL regions are linked with a peptide linker. Examples of peptide linkers that can be used to link various peptides provided for herein include, but are not limited to: (GGGGS)ₙ (SEQ ID NO: 35); (GGGGA)ₙ (SEQ ID NO: 36), or any combination thereof, wherein each n is independently 1-5. In some embodiments, the variable regions, e.g., the heavy chain variable region and light chain variable region, are not linked with a linker, e.g., a peptide linker, whereas in other embodiments, the variable regions, e.g., the heavy chain variable region and light chain variable region, are linked with a linker, e.g., a peptide linker

In some embodiments, the antibody comprises a heavy chain constant region of a class selected from IgG, IgA, IgD, IgE, and IgM. In some embodiments, the antibody comprises a heavy chain constant region of the class IgG and a subclass selected from IgG1, IgG2, IgG3, and IgG4.

In some embodiments, the antibody comprises an IgG1 heavy chain constant region. In some embodiments, the IgG1 heavy chain constant region comprises a wild type (WT) sequence. In some embodiments, the IgG1 heavy chain constant region comprises an M252Y, an S254T, and a T256E mutation or an M428L and a N434S mutation as numbered according to the EU numbering index. In some embodiments, the IgG1 heavy chain constant region comprises an M252Y, an S254T, and a T256E mutation as numbered according to the Kabat EU numbering index. In the heavy chain amino acid sequence disclosed herein as SEQ ID NO:9, which contains the M428L and a N434S mutations, the M428L mutation occurs at residue 432 of the heavy chain sequence and the N434S mutation occurs at residue 438 of the heavy chain sequence. In the heavy chain amino acid sequence disclosed herein as SEQ ID NO: 10, which contains the M252Y, an S254T, and a T256E mutations, the M252Y mutation occurs at residue 256 of the heavy chain sequence, S254T occurs at reside 258 of the heavy chain sequence, and T256E, occurs at residue 260 of the heavy chain sequence.

In some embodiments, the heavy chain comprises the amino acid sequence as set forth in SEQ ID NOs: 10, 9, or 7.

In some embodiments, the heavy chain comprises the amino acid sequence as set forth in SEQ ID NO: 10.

In some embodiments, the heavy chain comprises the amino acid sequence as set forth in SEQ ID NO: 9.

In some embodiments, the antibody, or antigen binding fragment thereof, comprises a peptide having a sequence as set forth in any of SEQ ID NOs: 1-10 and 11-19.

In some embodiments, an antibody, or antigen binding fragment thereof, comprises a sequence of, or a variant of any of the foregoing.

In some embodiments, the antibody, or antigen binding fragment thereof, comprises the amino acid sequence of SEQ ID NOs: 1, 3, 5, 7, 9, or 10, or a variant of any of the foregoing. In some embodiments, the antibody, or antigen binding fragment thereof, comprises the amino acid sequence of SEQ ID NOs: 2, 4, 6, or 8, or a variant of any of the foregoing.

In some embodiments, an antibody, or antigen binding fragment thereof, comprises a V_{L} peptide having the amino acid sequence of SEQ ID NO: 2, 4, 6, or 8, or any combination thereof. The V_{L} peptide can comprise a variant of any of these sequences as provided for herein.

In some embodiments, an antibody, or antigen binding fragment thereof, comprises a V_{H} peptide having the amino acid sequence of SEQ ID NO: 1, 3, 5, 7, 9, or 10, or any combination thereof. The V_{H} peptide can comprise a variant of any of these sequences as provided for herein.

In some embodiments, an antibody, or antigen binding fragment thereof, comprises a V_{H} peptide and a V_{L} peptide, wherein the V_{H} peptide comprises the sequence of SEQ ID NO: 1, 3, 5, 7, 9, or 10, and the V_{L} peptide comprises the sequence of SEQ ID NO: 2, 4, 6, or 8.

In some embodiments, an antibody, or antigen binding fragment thereof, comprises a V_{H} peptide and a V_{L} peptide, wherein the V_{H} peptide comprises a sequence that is at least 70%, 80%, 85%, 86%, 87%, 88%, 89%, 90%, 91%, 92%, 93%, 94%, 95%, 96%, 97%, 98%, or 99% identical to a sequence of SEQ ID NO: 1, 3, 5, 7, 9, or 10, and the V_{L} peptide comprises a sequence that is at least 85% 86% 87% 88% 89% 90% 91% 92% 93% 94% 95% 96% 97%, 98%, or 99% identical to a sequence of SEQ ID NO: 2, 4, 6, or 8.

In some embodiments, an antibody, or antigen binding fragment thereof, comprises a V_{H} peptide and a V_{L} peptide, wherein the V_{H} peptide comprises a sequence that is at least 85%, 86% 87% 88% 89% 90% 91% 92% 93% 94% 95% 96% 97% 98% or 99% identical to a sequence of SEQ ID NO: 1, 3, 5, 7, 9, or 10, and the V_{L} peptide comprises a sequence that is at least 85% 86% 87% 88% 89% 90% 91% 92% 93% 94% 95% 96% 97% 98% or 99% identical to a sequence of SEQ ID NO: 2, 4, 6, or 8, provided that the V_{L} peptide comprises a light chain CDR having the sequence of SEQ ID NO: 11, 12, 13, 18, 20, 21, 22, 26, 27, or 28 and the V_{H} peptide comprises a heavy chain CDR having the sequence of SEQ ID NO: 14, 15, 16, 17, 19, 23, 24, 25, 29, 30, 31, 43, or 44. In some embodiments, the CDRs in the V_{H} or V_{L} chain are as set forth in the combinations provided for herein.

In some embodiments, an antibody, or antigen binding fragment thereof, comprises a V_{H} peptide and a V_{L} peptide, wherein the V_{H} peptide comprises a sequence that is at least 85%, 86% 87% 88% 89% 90% 91% 92% 93% 94% 95% 96% 97% 98% or 99% identical to a sequence of SEQ ID NO: 1, 3, 5, 7, 9, or 10, and the V_{L} peptide comprises a sequence that is at least 85% 86% 87% 88% 89% 90% 91% 92% 93% 94% 95% 96% 97% 98% or 99% identical to a sequence of SEQ ID NO: 2, 4, 6, or 8, provided that the V_{L} peptide comprises an LCDR1 having a sequence selected from SEQ ID NO: 11, 20, or 27; an LCDR2 having a sequence selected from SEQ ID NO: 12, 18, 21, and 26; and an LCDR3 having a sequence selected from SEQ ID NO: 13, 22, and 28; and the V_{H} peptide comprises an HCDR1 having a sequence selected from SEQ ID NO: 14, 23, 29, or 43; a HCDR2 having a sequence selected from SEQ ID NO: 15, 17, 19, 24, 30, or 44; and an HCDR3 having a sequence selected from SEQ ID NO: 16, 25, or 31.

In some embodiments, an antibody, or antigen binding fragment thereof, comprises a V_{H} peptide and a V_{L} peptide, wherein the V_{H} peptide comprises a sequence that is at least 85%, 86% 87% 88% 89% 90% 91% 92% 93% 94% 95% 96% 97% 98% or 99% identical to a sequence of SEQ ID NO: 1, 3, 5, 7, 9, or 10, and the V_{L} peptide comprises a sequence that is at least 85% 86% 87% 88% 89% 90% 91% 92% 93% 94% 95% 96% 97% 98% or 99% identical to a sequence of SEQ ID NO: 2, 4, 6, or 8, provided that the V_{L} peptide comprises an LCDR1 having a sequence selected from SEQ ID NO: 11, 20, or 27, wherein the LCDR1 comprises at most 1 conservative amino acid substitution; an LCDR2 having a sequence selected from SEQ ID NO: 12, 18, 21, and 26, wherein the LCDR2 comprises at most 1 conservative amino acid substitution; and an LCDR3 having a sequence selected from SEQ ID NO: 13, 22, and 28, wherein the LCDR3 comprises at most 1 conservative amino acid substitution; and the V_{H} peptide comprises an HCDR1 having a sequence selected from SEQ ID NO: 14, 23, 29, or 43, wherein the HCDR1 comprises at most 1 conservative amino acid substitution; an HCDR2 having a sequence selected from SEQ ID NO: 15, 17, 19, 24, 30, or 44, wherein the HCDR2 comprises at most 1 conservative amino acid substitution; and an HCDR3 having a sequence selected from SEQ ID NO: 16, 25, or 31 wherein the HCDR3 comprises at most 1 conservative amino acid substitution.

In some embodiments, an antibody, or antigen binding fragment thereof, comprises a V_{H} peptide and a V_{L} peptide, wherein the V_{H} peptide comprises the sequence of SEQ ID NO: 1 and the V_{L} peptide comprises the sequence of SEQ ID NO: 2.

In some embodiments, an antibody, or antigen binding fragment thereof, comprises a V_{H} peptide and a V_{L} peptide, wherein the V_{H} peptide comprises the sequence of SEQ ID NO: 1 and the V_{L} peptide comprises the sequence of SEQ ID NO: 4, 6, or 8.

In some embodiments, an antibody, or antigen binding fragment thereof, comprises a V_{H} peptide and a V_{L} peptide, wherein the V_{H} peptide comprises a sequence of SEQ ID NO: 3 and the V_{L} peptide comprises a sequence of SEQ ID NO: 4.

In some embodiments, an antibody, or antigen binding fragment thereof, comprises a V_{H} peptide and a V_{L} peptide, wherein the V_{H} peptide comprises a sequence of SEQ ID NO: 3 and the V_{L} peptide comprises a sequence of SEQ ID NO: 2, 6, or 8.

In some embodiments, an antibody, or antigen binding fragment thereof, comprises a V_{H} peptide and a V_{L} peptide, wherein the V_{H} peptide comprises a sequence of SEQ ID NO: 5 and the V_{L} peptide comprises a sequence of SEQ ID NO: 6.

In some embodiments, an antibody, or antigen binding fragment thereof, comprises a V_{H} peptide and a V_{L} peptide, wherein the V_{H} peptide comprises a sequence of SEQ ID NO: 5 and the V_{L} peptide comprises a sequence of SEQ ID NO: 2, 4 or 8.

In some embodiments, an antibody, or antigen binding fragment thereof, comprises a V_{H} peptide and a V_{L} peptide, wherein the V_{H} peptide comprises a sequence of SEQ ID NO: 7 and the V_{L} peptide comprises a sequence of SEQ ID NO: 6.

In some embodiments, an antibody, or antigen binding fragment thereof, comprises a V_{H} peptide and a V_{L} peptide, wherein the V_{H} peptide comprises a sequence of SEQ ID NO: 7 and the V_{L} peptide comprises a sequence of SEQ ID NO: 8.

In some embodiments, an antibody, or antigen binding fragment thereof, comprises a V_{H} peptide and a V_{L} peptide, wherein the V_{H} peptide comprises a sequence of SEQ ID NO: 7 and the V_{L} peptide comprises a sequence of SEQ ID NO: 2 or 4

In some embodiments, an antibody, or antigen binding fragment thereof, comprises a V_{H} peptide and a V_{L} peptide, wherein the V_{H} peptide comprises a sequence of SEQ ID NO: 9 and the V_{L} peptide comprises a sequence of SEQ ID NO: 8.

In some embodiments, an antibody, or antigen binding fragment thereof, comprises a V_{H} peptide and a V_{L} peptide, wherein the V_{H} peptide comprises a sequence of SEQ ID NO: 9 and the V_{L} peptide comprises a sequence of SEQ ID NO: 6.

In some embodiments, an antibody, or antigen binding fragment thereof, comprises a V_{H} peptide and a V_{L} peptide, wherein the V_{H} peptide comprises a sequence of SEQ ID NO: 9 and the V_{L} peptide comprises a sequence of SEQ ID NO: 2, or 4.

In some embodiments, an antibody, or antigen binding fragment thereof, comprises a V_{H} peptide and a V_{L} peptide, wherein the V_{H} peptide comprises a sequence of SEQ ID NO: 10 and the V_{L} peptide comprises a sequence of SEQ ID NO: 8.

In some embodiments, an antibody, or antigen binding fragment thereof, comprises a V_{H} peptide and a V_{L} peptide, wherein the V_{H} peptide comprises a sequence of SEQ ID NO: 10 and the V_{L} peptide comprises a sequence of SEQ ID NO: 6.

In some embodiments, an antibody, or antigen binding fragment thereof, comprises a V_{H} peptide and a V_{L} peptide, wherein the V_{H} peptide comprises a sequence of SEQ ID NO: 10 and the V_{L} peptide comprises a sequence of SEQ ID NO: 2 or 4

In addition to these specific combinations, any of the V_{H} peptides disclosed herein can be combined with any of the V_{L} peptides disclosed herein.

As provided for herein, the different peptides (V_{H} or V_{L}) described herein can be linked with a peptide linker or not linked with a peptide linker and instead for a contiguous sequence. In some embodiments, the peptide linker comprises a sequence of (GGGGS)ₙ (SEQ ID NO: 35); (GGGGA)ₙ (SEQ ID NO: 36), or any combination thereof, wherein each n is independently 1-5. The linked peptide format can be represented by a formula of V_{H}—Z-V_{L} or V_{L}-Z-V_{H}, wherein Z is the peptide linker. In some embodiments, Z is (GGGGS)ₙ (SEQ ID NO: 35); (GGGGA)ₙ (SEQ ID NO: 36), or any combination thereof, wherein each n is independently 1-5.

### II. Antibody Epitope Binding

In some embodiments, the antibody binds to an epitope on human plasma kallikrein comprising at least one of residues K550 or 8551, of wild type human plasma kallikrein (SEQ ID NO: 32). In some embodiments, the antibody binds to an epitope on human plasma kallikrein comprising at least one of residues K585 or S597 of wild type human plasma kallikrein (SEQ ID NO: 32). In some embodiments, the antibody binds to an epitope on human plasma kallikrein comprising at least one of residues K585, H586, N587, G588, M589, W590, R591, L592, V593, G594, I595, T596, or S597 of wild type human plasma kallikrein (SEQ ID NO: 32). In some embodiments, the antibody binds to an epitope on human plasma kallikrein comprising at least one of residues Y617 or T625 of wild type human plasma kallikrein (SEQ ID NO: 32). In some embodiments, the antibody binds to an epitope on human plasma kallikrein comprising at least one of residues Y617, M618, D619, W620, I621, L622, E623, K624, or T625 of wild type human plasma kallikrein (SEQ ID NO: 32). In some embodiments, the antibody binds to an epitope on human plasma kallikrein comprising at least one of residues K550, R551, K585, S597, Y617 or T625 of wild type human plasma kallikrein (SEQ ID NO: 32). In some embodiments, the antibody binds to an epitope on human plasma kallikrein comprising at least one of residues K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least one of the following residues: K550, R551, K585, S597, Y617, or T625 of human plasma kallikrein listed in SEQ ID NO: 32.

In some embodiments, the antibody or antigen binding fragment thereof binds to at least two of the following residues: K550, R551, K585, S597, Y617, or T625 of human plasma kallikrein listed in SEQ ID NO: 32.

In some embodiments, the antibody or antigen binding fragment thereof binds to at least three of the following residues: K550, R551, K585, S597, Y617, or T625 of human plasma kallikrein listed in SEQ ID NO: 32.

In some embodiments, the antibody or antigen binding fragment thereof binds to at least four of the following residues: K550, R551, K585, S597, Y617, or T625 of human plasma kallikrein listed in SEQ ID NO: 32.

In some embodiments, the antibody or antigen binding fragment thereof binds to at least five of the following residues: K550, R551, K585, S597, Y617, or T625 of human plasma kallikrein listed in SEQ ID NO: 32.

In some embodiments, the antibody or antigen binding fragment thereof binds to the following residues: K550, R551, K585, S597, Y617, or T625 of human plasma kallikrein listed in SEQ ID NO: 32.

In some embodiments, the antibody or antigen binding fragment thereof binds to at least one of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least two, three, four, five, six, seven, eight, nine, ten, eleven, twelve, thirteen, fourteen, fifteen, sixteen, seventeen, eighteen, nineteen, twenty, twenty-one, twenty-two, twenty-three, or twenty-four of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least two of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least three of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least four of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least five of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least six of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least seven of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least eight of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least nine of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least ten of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least eleven of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least twelve of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least thirteen of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least fourteen of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least fifteen of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least sixteen of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least seventeen of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least eighteen of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least nineteen of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least twenty of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least twenty-one of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least twenty-two of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least twenty-three of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least twenty-four of the following residues: K550, R551, K585, H586, N587, G588, M589, W590, R591, L592, V593, G594, I595, T596, S597, Y617, M618, D619, W620, I621, L622, E623, K624, or T625 of wild type human plasma kallikrein (SEQ ID NO: 32).

In some embodiments, the antibody or antigen binding fragment thereof binds to at least K550 of wild type human plasma kallikrein (SEQ ID NO: 32). In some embodiments, the antibody or antigen binding fragment thereof binds to at least R551 of wild type human plasma kallikrein (SEQ ID NO: 32). In some embodiments, the antibody or antigen binding fragment thereof binds to at least K585 of wild type human plasma kallikrein (SEQ ID NO: 32). In some embodiments, the antibody or antigen binding fragment thereof binds to at least H586 of wild type human plasma kallikrein (SEQ ID NO: 32). In some embodiments, the antibody or antigen binding fragment thereof binds to at least N587 of wild type human plasma kallikrein (SEQ ID NO: 32). In some embodiments, the antibody or antigen binding fragment thereof binds to at least G588 of wild type human plasma kallikrein (SEQ ID NO: 32). In some embodiments, the antibody or antigen binding fragment thereof binds to at least M589 of wild type human plasma kallikrein (SEQ ID NO: 32). In some embodiments, the antibody or antigen binding fragment thereof binds to at least W590 of wild type human plasma kallikrein (SEQ ID NO: 32). In some embodiments, the antibody or antigen binding fragment thereof binds to at least R591 of wild type human plasma kallikrein (SEQ ID NO: 32). In some embodiments, the antibody or antigen binding fragment thereof binds to at least L592 of wild type human plasma kallikrein (SEQ ID NO: 32). In some embodiments, the antibody or antigen binding fragment thereof binds to at least V593 of wild type human plasma kallikrein (SEQ ID NO: 32). In some embodiments, the antibody or antigen binding fragment thereof binds to at least G594 of wild type human plasma kallikrein (SEQ ID NO: 32). In some embodiments, the antibody or antigen binding fragment thereof binds to at least I595 of wild type human plasma kallikrein (SEQ ID NO: 32). In some embodiments, the antibody or antigen binding fragment thereof binds to at least T596 of wild type human plasma kallikrein (SEQ ID NO: 32). In some embodiments, the antibody or antigen binding fragment thereof binds to at least S597 of wild type human plasma kallikrein (SEQ ID NO: 32). In some embodiments, the antibody or antigen binding fragment thereof binds to at least Y617 of wild type human plasma kallikrein (SEQ ID NO: 32). In some embodiments, the antibody or antigen binding fragment thereof binds to at least M618 of wild type human plasma kallikrein (SEQ ID NO: 32). In some embodiments, the antibody or antigen binding fragment thereof binds to at least D619 of wild type human plasma kallikrein (SEQ ID NO: 32). In some embodiments, the antibody or antigen binding fragment thereof binds to at least W620 of wild type human plasma kallikrein (SEQ ID NO: 32). In some embodiments, the antibody or antigen binding fragment thereof binds to at least I621 of wild type human plasma kallikrein (SEQ ID NO: 32). In some embodiments, the antibody or antigen binding fragment thereof binds to at least L622 of wild type human plasma kallikrein (SEQ ID NO: 32). In some embodiments, the antibody or antigen binding fragment thereof binds to at least E623 of wild type human plasma kallikrein (SEQ ID NO: 32). In some embodiments, the antibody or antigen binding fragment thereof binds to at least K624 of wild type human plasma kallikrein (SEQ ID NO: 32). In some embodiments, the antibody or antigen binding fragment thereof binds to at least T625 of wild type human plasma kallikrein (SEQ ID NO: 32).

In some embodiments, the antibody or antigen binding fragment thereof binds to a linear epitope of human plasma kallikrein. For example, the linear epitope has the amino acid sequence of SEQ ID NO: 37, 38, or 39. The antibody or antigen binding fragment thereof may bind to one, two or three of these linear epitopes. In further embodiments, the antibody or antigen binding fragment does not bind to linear epitopes having the sequences of SEQ ID NO:40, 41, and/or 42, and/or any amino acid residue of the amino acid sequences of SEQ ID NO:40, 41, and/or 42.

### III. Antibody Conjugates

The antibodies provided for herein may also be conjugated to a chemical moiety. The chemical moiety may be, inter al/a, a polymer, a radionuclide or a cytotoxic factor. The chemical moiety may be an MRI-detectable label. In some embodiments, this can be referred to as an antibody drug conjugate. In some embodiments, the chemical moiety is a polymer which increases the half-life of the antibody molecule in the body of a subject. Suitable polymers include, but are not limited to, polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee, et al., (1999) (Bioconj. Chem. 10:973-981) discloses PEG conjugated single-chain antibodies. Wen, et al., (2001) (Bioconj. Chem. 12:545 553) disclose conjugating antibodies with PEG which is attached to a radiometal chelator (diethylenetriaminpentaacetic acid (DTPA)). Examples of chemical moieties include, but are not limited to, anti-mitotics, such as calicheamicins (e.g. ozogamicin), monomethyl auristatin E, mertansine, and the like. Other examples include, but are not limited to, biologically active anti-microtubule agents, alkylating agents and DNA minor groove binding agents. The chemical moiety can be linked to the antibody through a linking group (maleimide), a cleavable linker, such as a cathepsin cleavable linkers (valine-citrulline), and in some embodiments, one or more spacers (e.g. para-aminobenzylcarbamate).

The antibodies and antibody fragments of the invention may also be conjugated with labels such as ⁹⁹Tc, ⁹⁰Y, ¹¹¹In, ³²P, ¹⁴C, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰Co ⁵⁹Fe, ⁵⁷Se, ¹⁵²Eu, ⁶⁷CU, ²¹⁷Ci, ²¹¹At, ²¹²Pb, ⁴⁷Sc, ¹⁰⁹Pd, ²³⁴Th, and ⁴⁰K, ¹⁵⁷Gd, ⁵⁵Mn, ⁵²Tr and ⁵⁶Fe.

The antibodies and antibody fragments may also be conjugated with fluorescent or chemiluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

The antibody molecules may also be conjugated to a cytotoxic factor such as diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, Momordica charantia inhibitor, curcin, cretin, Saponaria officinalis inhibitor, mitogellin, restrictocin, phenomycin, and enomycin.

Any method known in the art for conjugating the antibody molecules of the invention to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating antibodies are conventional and very well known in the art.

In some embodiments, antibodies (e.g. an anti-plasma kallikrein antibody) are provided herein. In some embodiments, the antibody is a recombinant antibody that binds to plasma kallikrein. In some embodiments, the plasma kallikrein protein is a human plasma kallikrein protein. In some embodiments, the antibody does not specifically bind to prekallikrein protein. As used herein, the term “recombinant antibody” refers to an antibody that is not naturally occurring. In some embodiments, the term “recombinant antibody” refers to an antibody that is not isolated from a human subject.

### IV. Nucleic Acids and Methods of Production

The invention also contemplates nucleic acids encoding each of the antibodies or antigen binding fragments disclosed herein, including nucleic acids encoding a heavy chain or a light chain, or variable regions of the heavy or light chain of such antibodies as disclosed herein. Accordingly, in some embodiments, a nucleic acid molecule encoding an antibody or fragment as disclosed herein is provided. In some embodiments, the nucleic acid encodes an amino acid sequence of an antibody or antigen binding fragment thereof, or of a heavy or light chain, or heavy or light chain variable region provided herein.

For example, in one embodiment, a nucleic acid encodes the heavy chain variable region of any one of SEQ ID NOs: 1, 3, or 5, while in another embodiment, a nucleic acid encodes the light chain variable region of any one of SEQ ID NOs: 2, 4, or 6. In certain embodiments, a nucleic acid encodes the heavy chain sequence of SEQ ID NOs: 7, 9, or 10, while in another, a nucleic acid encodes the light chain sequence of SEQ ID NO:8.

In one embodiment, a nucleic acid encodes a heavy chain or a heavy chain variable region comprising an HCDR1 having the amino acid sequence of SEQ ID NOs: 14, 23, 29, or 43; an HCDR2 having the amino acid sequence of SEQ ID NOs: 15, 17, 19, 24, 30, or 44; and an HCDR3 having the amino acid sequence of SEQ ID NOs: 16, 25, or 31.

In another embodiment, a nucleic acid encodes a light chain or a light chain variable region comprising an LCDR1 having the amino acid sequence of SEQ ID NOs: 11, 20, or 27; an LCDR2 having the amino acid sequence of SEQ ID NOs: 12, 18, 21, or 26; and an LCDR3 having the amino acid sequence of SEQ ID NOs: 13, 22, or 28.

In another embodiment, an antibody of the invention comprises a heavy chain variable region encoded by the nucleic acid sequence of SEQ ID NO: 33 and a light chain variable region encoded by the nucleic acid sequence of SEQ ID NO: 34. An antibody of the invention may also comprise a heavy chain encoded by the nucleic acid sequence of SEQ ID NO: 48 and a light chain encoded by the nucleic acid sequence of SEQ ID NO: 49.

As described herein the production of antibodies with a known sequence is routine and can be done by any method. Methods for producing antibodies, such as those disclosed herein, are known in the art. Once the sequences are known, the antibodies can also be generated according to known methods, including according the examples provided herein.

For example, DNA molecules encoding light chain variable regions and/or heavy chain variable regions can be chemically synthesized using the sequence information provided herein. Synthetic DNA molecules can be ligated to other appropriate nucleotide sequences, including, e.g., constant region coding sequences, and expression control sequences, to produce conventional gene expression constructs encoding the desired antibodies. Production of defined gene constructs is within routine skill in the art. Alternatively, the sequences provided herein can be cloned out of hybridomas by conventional hybridization techniques or polymerase chain reaction (PCR) techniques, using synthetic nucleic acid probes whose sequences are based on sequence information provided herein, or prior art sequence information regarding genes encoding the heavy and light chains of murine antibodies in hybridoma cells.

Nucleic acids encoding desired antibodies can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the immunoglobulin light and/or heavy chain variable regions. Accordingly, the invention provides vectors comprising nucleic acids encoding the antibodies or fragments thereof of the invention, as well as cells comprising such nucleic acids or vectors.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed secreted protein accumulates in refractile or inclusion bodies, and can be harvested after disruption of the cells by French press or sonication. The refractile bodies then are solubilized, and the proteins refolded and cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. This expression vector optionally contains sequences encoding all or part of a constant region, enabling an entire, or a part of, a heavy or light chain to be expressed. The gene construct can be introduced into eukaryotic host cells using conventional techniques. The host cells express V_{L} or V_{H} fragments, V_{L}-V_{H} heterodimers, V_{H}-V_{L} or V_{L}-V_{H} single chain polypeptides, complete heavy or light immunoglobulin chains, or portions thereof, each of which may be attached to a moiety having another function (e.g., cytotoxicity). In some embodiments, a host cell is transfected with a single vector expressing a polypeptide expressing an entire, or part of, a heavy chain (e.g., a heavy chain variable region) or a light chain (e.g., a light chain variable region). In some embodiments, a host cell is transfected with a single vector encoding (a) a polypeptide comprising a heavy chain variable region and a polypeptide comprising a light chain variable region, or (b) an entire immunoglobulin heavy chain and an entire immunoglobulin light chain. In some embodiments, a host cell is co-transfected with more than one expression vector (e.g., one expression vector expressing a polypeptide comprising an entire, or part of, a heavy chain or heavy chain variable region, and another expression vector expressing a polypeptide comprising an entire, or part of, a light chain or light chain variable region).

A polypeptide comprising an immunoglobulin heavy chain variable region or light chain variable region can be produced by growing (culturing) a host cell transfected with an expression vector encoding such a variable region, under conditions that permit expression of the polypeptide. Following expression, the polypeptide can be harvested and purified or isolated using techniques known in the art, e.g., affinity tags such as glutathione-S-transferase (GST) or histidine tags.

A monoclonal antibody that binds plasma kallikrein, or an antigen-binding fragment of the antibody, can be produced by growing (culturing) a host cell transfected with: (a) an expression vector that encodes a complete or partial immunoglobulin heavy chain, and a separate expression vector that encodes a complete or partial immunoglobulin light chain; or (b) a single expression vector that encodes both chains (e.g., complete or partial heavy and light chains), under conditions that permit expression of both chains. The intact antibody (or antigen-binding fragment) can be harvested and purified or isolated using techniques known in the art, e.g., Protein A, Protein G, affinity tags such as glutathione-S-transferase (GST) or histidine tags. It is within ordinary skill in the art to express the heavy chain and the light chain from a single expression vector or from two separate expression vectors.

The nucleic acid sequence encoding an antibody described herein can be genomic DNA or cDNA, or RNA (e.g. mRNA) which encodes at least one of the variable regions described herein. A convenient alternative to the use of chromosomal gene fragments as the source of DNA encoding the V region antigen-binding segment is the use of cDNA for the construction of chimeric immunoglobulin genes, e.g., as reported by Liu et al. (Proc. Natl. Acad. Sci., USA 84:3439 (1987) and J. Immunology 139:3521 (1987), which references are hereby entirely incorporated herein by reference. The use of cDNA requires that gene expression elements appropriate for the host cell be combined with the gene in order to achieve synthesis of the desired protein. The use of cDNA sequences is advantageous over genomic sequences (which contain introns), in that cDNA sequences can be expressed in bacteria or other hosts which lack appropriate RNA splicing systems.

For example, a cDNA encoding a V region antigen-binding segment able to detect, bind, to or neutralize plasma kallikrein can be provided using known methods based on the use of the amino acid sequences provided herein. Because the genetic code is degenerate, more than one codon can be used to encode a particular amino acid (Watson, et al., infra). Using the genetic code, one or more different oligonucleotides can be identified, each of which would be capable of encoding the amino acid. The probability that a particular oligonucleotide will, in fact, constitute the actual XXX-encoding sequence can be estimated by considering abnormal base pairing relationships and the frequency with which a particular codon is actually used (to encode a particular amino acid) in eukaryotic or prokaryotic cells expressing an antibody or fragment. Such “codon usage rules” are disclosed by Lathe, et al., J. Molec. Biol. 183:1 12 (1985). Using the “codon usage rules” of Lathe, a single oligonucleotide, or a set of oligonucleotides, that contains a theoretical “most probable” nucleotide sequence capable of encoding an antibody variable or constant region sequences is identified.

V. Pharmaceutical Compositions and their Administration

The plasma kallikrein binding proteins disclosed herein, including antibodies and antigen binding fragments thereof, and including nucleic acid molecules encoding the same, can be formulated as pharmaceutical compositions, e.g., for administration to a subject.

As used herein, the term “pharmaceutical composition” refers to the combination of an active agent or agents (such as an antibody or antigen binding fragment described herein) with a carrier (inert or active) that is suitable for diagnostic or therapeutic use in vivo or ex vivo.

Pharmaceutical compositions containing antibodies, fragments thereof, or nucleic acids encoding such antibodies, as disclosed herein, can be presented in a dosage unit form and can be prepared by any suitable method. In some embodiments, to prepare pharmaceutical or sterile compositions of the anti-plasma kallikrein antibodies or other proteins provided herein, the antibody or antigen binding fragment thereof or other proteins provided herein are admixed with a pharmaceutically acceptable carrier or excipient. A pharmaceutical composition should be formulated to be compatible with its intended route of administration. Useful formulations can be prepared by methods known in the pharmaceutical art. For example, see Remington's Pharmaceutical Sciences, 18th ed. (Mack Publishing Company, 1990).

The phrases “pharmaceutically acceptable” and “pharmacologically acceptable,” as used herein, refer to compounds, molecular entities, compositions, materials, and/or dosage forms that do not produce an adverse, allergic or other untoward reaction when administered to an animal, or a human, as appropriate. For human administration, preparations should meet sterility, pyrogenicity, and general safety and purity standards as required by FDA Office of Biologics standards. “Pharmaceutically acceptable” and “pharmacologically acceptable” can mean approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

As used herein, “carrier” refers to a material, composition or vehicle, such as a liquid or solid filler, diluent, excipient, solvent or encapsulating material, involved in carrying or transporting a pharmaceutical agent such as an antibody or binding fragment thereof from one organ, or portion of the body, to another organ, or portion of the body.

As used herein, “pharmaceutically acceptable excipient” refers to a substance that aids the administration of an active agent, e.g., an antibody or binding fragment thereof, to and/or absorption by a subject and can be included in the compositions of the present invention without causing a significant adverse toxicological effect on the patient. Non-limiting examples of pharmaceutically acceptable excipients include water, NaCl, normal saline solutions, such as a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, dextrose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compositions of the invention. For examples of excipients, see Martin, Remington's Pharmaceutical Sciences, 15th Ed., Mack Publ. Co., Easton, PA (1975).

Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, NJ), phosphate buffered saline (PBS) or dextrose solution. The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyethylene glycol), and suitable mixtures thereof.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

Formulations of the antibodies provided herein may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY). In some embodiments, the antibodies are diluted to an appropriate concentration in a sodium acetate solution pH 5-6, and NaCl or sucrose is added for tonicity. Additional agents, such as polysorbate 20 or polysorbate 80, may be added to enhance stability.

Toxicity and therapeutic efficacy of the antibody compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, antibodies exhibiting high therapeutic indices are desirable. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

Antibody compositions of the invention may be administered to a subject, for example, in accordance with the Physicians' Desk Reference 2003 (Thomson Healthcare; 57th edition (Nov. 1, 2002)).

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial.

In some embodiments, the antibody or antigen binding fragment thereof can be administered by an invasive route such as by injection. In some embodiments, the antibodies or antigen binding fragment thereof, or pharmaceutical composition thereof, is administered intravenously, subcutaneously, intramuscularly, intraarterially, intra-articularly (e.g. in arthritis joints), intratumorally, or by inhalation, aerosol delivery. Administration by non-invasive routes (e.g., orally; for example, in a pill, capsule or tablet) is also within the scope of the present embodiments. Administration by intravenous infusion is one manner in which the antibodies may be administered to a subject.

In some embodiments, the anti-plasma kallikrein antibody, or antigen binding fragment thereof, is administered in combination with at least one additional therapeutic agent, such as, but not limited to any therapeutic used to treat the disorders provided for herein. In some embodiments, the antibody is administered in combination with another treatment for the disorders provided for herein. For example, the anti-plasma kallikrein antibody of the invention may be administered with one or more additional therapies used to treat HAE, such as BERINERT® (C1 esterase inhibitor), FIRAZYR® (icatibant injection), KALBITOR® (ecallantide), and RUCONEST® (C1 esterase inhibitor, contestat alfa), CINRYZE® (C1 esterase inhibitor), HAEGARDA® (C1 esterase inhibitor subcutaneous), TAKHZYRO® (lanadelumab-flyo), ORLADEYO™ (berotralstat), garadacimab, donidalorsen, synthetic 17-α-alkylated androgens such as danazol and stanozolol, and/or antifibrinolytic agents such as aminocaproic acid. The anti-plasma kallikrein antibody may be administered simultaneously with one or more aforementioned additional therapies, e.g., they may be co-administered. The anti-plasma kallikrein antibody may be administered sequentially, either before or after, one of the aforementioned therapies, for example, on different days or different weeks or even in different months than any of the aforementioned additional therapies.

Compositions can be administered with medical devices known in the art. For example, a pharmaceutical composition of the invention can be administered by injection with a hypodermic needle, including, e.g., a prefilled syringe or autoinjector.

The pharmaceutical compositions may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556.

The pharmaceutical compositions may also be administered by infusion. Examples of well-known implants and modules form administering pharmaceutical compositions include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art. Infusion may also be carried out by intravenous delivery of an antibody in a pharmaceutically acceptable carrier, such as physiologic saline, over a defined time period. Intravenous delivery of the pharmaceutical composition may occur via a port implanted into a vein or artery in the subject.

Alternately, one may administer the antibody in a local rather than systemic manner, for example, via injection or in a depot or sustained release formulation. Furthermore, one may administer the antibody in a targeted drug delivery system, for example, in a liposome coated with a tissue-specific antibody.

The administration regimen depends on several factors, including the serum or tissue turnover rate of the therapeutic antibody, the level of symptoms, the immunogenicity of the therapeutic antibody, and the accessibility of the target cells in the biological matrix. Preferably, the administration regimen delivers sufficient therapeutic antibody to effect improvement in the target disease state, while simultaneously minimizing undesired side effects. Accordingly, the amount of biologic delivered depends in part on the particular therapeutic antibody and the severity of the condition being treated. Guidance in selecting appropriate doses of therapeutic antibodies is available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert, et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602).

Determination of the appropriate dose can be made by the clinician, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent. In the case of human subjects, for example, chimeric, humanized and fully human antibodies are may be desirable.

Antibodies or antigen binding fragments thereof can be provided by continuous infusion, or by doses administered, e.g., daily, 1-7 times per week, weekly, bi-weekly, monthly, bimonthly, or quarterly. In some embodiments, a total weekly dose is generally at least 0.05 μg/kg body weight, more generally at least 0.2 μg/kg, 0.5 μg/kg, 1 μg/kg, 10 μg/kg, 100 μg/kg, 0.25 mg/kg, 1.0 mg/kg, 2.0 mg/kg, 5.0 mg/ml, 10 mg/kg, 25 mg/kg, 50 mg/kg or more (see, e.g., Yang, et al. (2003) New Engl. J. Med. 349:427-434; Herold, et al. (2002) New Engl. J. Med. 346:1692-1698; Liu, et al. (1999) J. Neurol. Neurosurg. Psych. 67:451-456; Portielji, et al. (2003) Cancer Immunol. Immunother. 52:133-144). Doses may also be provided to achieve a pre-determined target concentration of the antibody in the subject's serum, such as 0.1, 0.3, 1, 3, 10, 30, 100, 300 μg/ml or more. In other embodiments, an antibody is administered subcutaneously or intravenously, on a weekly, biweekly, “every 4 weeks,” monthly, bimonthly, or quarterly basis at 10, 20, 50, 80, 100, 200, 500, 1000, 1500, 2000, or 2500 mg/subject.

### VI. Methods of Treatment and Pharmaceutical Uses

The plasma kallikrein binding proteins disclosed herein, including antibodies and antigen binding fragments thereof, can be used to treat disease in a subject.

As used herein, the terms “treat” or “treatment” includes inhibition or postponement of the development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms.

As used herein, the terms “therapeutically effective amount”, “therapeutically effective dose” and “effective amount” refer to an amount of the antibody, or antigen binding fragment thereof, that, when administered alone or in combination with an additional therapeutic agent to a cell, tissue, or subject, is effective to cause a measurable improvement in one or more symptoms of a disease or condition or the progression of such disease or condition, e.g. HAE or a condition in which plasma kallikrein is known to have caused the pathology observed. A therapeutically effective dose further refers to that amount of the antibody or binding fragment thereof sufficient to result in at least partial amelioration of symptoms, e.g., treatment, healing, prevention or amelioration of the relevant medical condition, e.g., HAE or a condition in which plasma kallikrein is known to have caused the pathology observed, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity. In some embodiments, an amount is a therapeutically effective amount if it is an amount that can be used to treat or prevent symptoms of HAE, or a condition in which plasma kallikrein is known to have caused the pathology observed. For example, a therapeutically effective amount of antibodies or binding fragments thereof of the invention may reduce the frequency of an HAE attack, or may reduce the severity of an HAE attack, for example, the level of edema experienced by the subject during an attack.

The term “subject,” as used herein, refers to any organism, such as an animal, including a mammal (e.g., rat, mouse, dog, cat, rabbit) and, for example, a human. In one embodiment, the subject is a human. A subject can be also be referred to as a patient. In some embodiments, the subject is a subject in need thereof. A subject that is “in need thereof” refers to a subject that has been identified as requiring treatment for the condition that is to be treated and is treated with the specific intent of treating such condition. The conditions can be, for example, any of the conditions described herein. “Subject” is used interchangeably with “patient.”

In some embodiments, the methods comprise administering a therapeutically or prophylactically effective amount of one or more antibodies or antigen binding fragments of the antibodies described herein or a pharmaceutical composition containing the therapeutically effective amount to a susceptible subject or to one exhibiting a condition in which plasma kallikrein is known to have caused the pathology observed. Any active form of the antibody can be administered, including, but not limited to scFv, Fab and F(ab′)₂ fragments and other forms of antibodies provided for herein.

As used herein, “a plasma kallikrein associated pathology” refers to conditions that are caused by the function or aberrant expression of plasma kallikrein. These conditions include, but are not limited to, hereditary angioedema, bradykinin dependent edema, diabetic macular edema, retinal edema, Factor XII-associated cold autoinflammatory syndrome (FACAS), rheumatoid arthritis, gout, intestinal bowel disease, oral mucositis, neuropathic pain, inflammatory pain, spinal stenosis-degenerative spine disease, arterial or venous thrombosis, post-operative ileus, aortic aneurysm, osteoarthritis, vasculitis, edema, cerebral edema, pulmonary embolism, stroke, clotting induced by ventricular assistance devices or stents, head trauma or pen-tumor brain edema, sepsis, acute middle cerebral artery (MCA) ischemic event (stroke), restenosis (e.g., after angioplasty), systemic lupus erythematosus nephritis, and burn injury. In some embodiments, the pathology is hereditary angioedema. In some embodiments, the pathology is bradykinin dependent edema.

In some embodiments, the antibodies provided herein are used to treat a subject who has or is suspected of having a plasma kallikrein associated disorder. In some embodiments, the plasma kallikrein associated disorder is selected from the group consisting of hereditary angioedema, bradykinin dependent edema, diabetic macular edema, retinal edema, Factor XII-associated cold autoinflammatory syndrome (FACAS), rheumatoid arthritis, gout, intestinal bowel disease, oral mucositis, neuropathic pain, inflammatory pain, spinal stenosis-degenerative spine disease, arterial or venous thrombosis, post-operative ileus, aortic aneurysm, osteoarthntis, vasculitis, edema, cerebral edema, pulmonary embolism, stroke, clotting induced by ventricular assistance devices or stents, head trauma or peni-tumor brain edema, sepsis, acute middle cerebral artery (MCA) ischemic event (stroke), restenosis (e.g., after angioplasty), systemic lupus erythematosus nephritis, and burn injury.

In some embodiments, the plasma kallikrein binding protein reduces aberrant clotting associated with the contact activation system (i.e., intrinsic activation system) by at least 10% as measured by e.g., an APTT clotting assay (e.g., by at least 20%, at least 30%, at least 40%, at least 50%, at least 60%, at least 70%, at least 80%, at least 90%, at least 95%, at least 99%, or even 100% (i.e., no detectable aberrant clotting)).

In some embodiments, the antibodies provided herein can be used for the treatment of a disorder described herein, e.g., hereditary angioedema, diabetic macular edema, retinal edema, Factor XII-associated cold autoinflammatory syndrome (FACAS), rheumatoid arthritis, gout, intestinal bowel disease, oral mucositis, neuropathic pain, inflammatory pain, spinal stenosis-degenerative spine disease, arterial or venous thrombosis, post-operative ileus, aortic aneurysm, osteoarthritis, vasculitis, edema, hereditary angioedema, cerebral edema, pulmonary embolism, stroke, clotting induced by ventricular assistance devices or stents, head trauma or peri-tumor brain edema, sepsis, acute middle cerebral artery (MCA) ischemic event (stroke), restenosis (e.g., after angioplasty), systemic lupus erythematosus nephritis, or burn injury; or to promote wound healing. In some embodiments, the disorder is hereditary angioedema or bradykinin dependent edema. In some embodiments, the disorder is hereditary angioedema.

In some embodiments, the antibodies provided for herein are used for the manufacture of a medicament for the treatment of a disorder described herein, e.g., hereditary angioedema, diabetic macular edema, retinal edema, Factor XII-associated cold autoinflammatory syndrome (FACAS), rheumatoid arthritis, gout, intestinal bowel disease, oral mucositis, neuropathic pain, inflammatory pain, spinal stenosis-degenerative spine disease, arterial or venous thrombosis, post-operative ileus, aortic aneurysm, osteoarthritis, vasculitis, edema, hereditary angioedema, cerebral edema, pulmonary embolism, stroke, clotting induced by ventricular assistance devices or stents, head trauma or peri-tumor brain edema, sepsis, acute middle cerebral artery (MCA) ischemic event (stroke), restenosis (e.g., after angioplasty), systemic lupus erythematosus nephritis, or burn injury; or for the manufacture of a medicament for wound healing.

Treatment of individuals may comprise the administration of a therapeutically effective amount of the antibodies or antigen binding fragments described herein, or pharmaceutical compositions containing such antibodies or fragments. The antibodies can be provided in a kit, such as those provided herein. The antibodies can be used or administered alone or in admixture with another therapeutic, analgesic, or diagnostic agent, such as provided for herein. In providing a patient with an antibody, or fragment thereof, capable of binding to plasma kallikrein, or an antibody capable of protecting against plasma kallikrein in a recipient patient, the dosage of administered agent will vary depending upon such factors as the patient's age, weight, height, sex, general medical condition, previous medical history, etc.

An antibody capable of treating a condition associated with plasma kallikrein activity or used to treat a plasma kallikrein related pathology, is intended to be provided to subjects in an amount sufficient to affect a reduction, resolution, or amelioration in the plasma kallikrein related symptom or pathology. Such a pathology includes, but is not limited to hereditary angioedema, bradykinin dependent edema, hereditary angioedema, diabetic macular edema, retinal edema, Factor XII-associated cold autoinflammatory syndrome (FACAS), rheumatoid arthritis, gout, intestinal bowel disease, oral mucositis, neuropathic pain, inflammatory pain, spinal stenosis-degenerative spine disease, arterial or venous thrombosis, post-operative ileus, aortic aneurysm, osteoarthritis, vasculitis, edema, cerebral edema, pulmonary embolism, stroke, clotting induced by ventricular assistance devices or stents, head trauma or pen-tumor brain edema, sepsis, acute middle cerebral artery (MCA) ischemic event (stroke), restenosis (e.g., after angioplasty), systemic lupus erythematosus nephritis, and burn injury. For example, the pathology is hereditary angioedema (HAE).

Accordingly, in some embodiments, methods of treating a subject with a plasma kallikrein mediated disorder are provided. In some embodiments, the method comprises administering a pharmaceutical composition comprising a therapeutically effective amount of an antibody, or antigen binding fragment thereof, as provided herein. In some embodiments, the disorder is hereditary angioedema, bradykinin dependent edema, hereditary angioedema, diabetic macular edema, retinal edema, Factor XII-associated cold autoinflammatory syndrome (FACAS), rheumatoid arthritis, gout, intestinal bowel disease, oral mucositis, neuropathic pain, inflammatory pain, spinal stenosis-degenerative spine disease, arterial or venous thrombosis, post-operative ileus, aortic aneurysm, osteoarthritis, vasculitis, edema, cerebral edema, pulmonary embolism, stroke, clotting induced by ventricular assistance devices or stents, head trauma or peri-tumor brain edema, sepsis, acute middle cerebral artery (MCA) ischemic event (stroke), restenosis (e.g., after angioplasty), systemic lupus erythematosus nephritis, and burn injury. In some embodiments, the disorder is hereditary angioedema. In some embodiments, the disorder is bradykinin dependent edema.

Methods of reducing or inhibiting plasma kallikrein activity in a subject in need thereof are also disclosed herein. For example, the antibodies or binding fragments thereof disclosed herein can be administered to a subject in a therapeutically effective amount to reduce or inhibit the activity of plasma kallikrein, such as human plasma kallikrein in that subject. The subject may have a plasma kallikrein associated disorder as disclosed herein, such as HAE. In some embodiments, plasma kallikrein activity may be reduced or inhibited in an amount sufficient to prevent an HAE attack in the subject for at least one month, two months, three months, four months, five months, six months, or more.

Methods of reducing or inhibiting bradykinin production or activity in a subject in need thereof are also disclosed herein. For example, the antibodies or binding fragments disclosed herein can be administered to a subject in a therapeutically effective amount to reduce the level or activity of bradykinin in the subject. The subject may have a plasma kallikrein associated disorder as disclosed herein, such as HAE. In some embodiments, bradykinin production or activity may be reduced or inhibited in an amount to prevent an HAE attack in the subject for at least one month, two months, three months, four months, five months, six months, or more.

As provided for herein, the antibodies, or antigen binding fragments thereof, can be administered with other therapeutics, which may include one or more additional therapies used to treat HAE, such as BERINERT® (C1 esterase inhibitor), FIRAZYR® (icatibant injection), KALBITOR® (ecallantide), and RUCONEST® (C1 esterase inhibitor, contestat alfa), CINRYZE® (C1 esterase inhibitor), HAEGARDA® (C1 esterase inhibitor subcutaneous), TAKHZYRO® (lanadelumab-flyo), ORLADEYO™ (berotralstat), garadacimab, donidalorsen, synthetic 17-α-alkylated androgens such as danazol and stanozolol, and/or antifibrinolytic agents such as aminocaproic acid. The additional therapy(ies) can be administered simultaneously or sequentially with the antibodies disclosed herein.

Kits are also provided which are useful for carrying out embodiments described herein. The present kits can comprise a first container containing or packaged in association with the above-described antibodies. The kit may also comprise another container containing or packaged in association solutions necessary or convenient for carrying out the embodiments. The containers can be made of glass, plastic or foil and can be a vial, bottle, pouch, tube, bag, etc. The kit may also contain written information, such as procedures for carrying out the embodiments or analytical information, such as the amount of reagent contained in the first container means. The container may be in another container apparatus, e.g. a box or a bag, along with the written information.

In some embodiments, antibodies that bind to a plasma kallikrein protein are provided. In some embodiments, the antibody is isolated. In some embodiments, the antibody binds specifically to plasma kallikrein.

In some embodiments, the antibody inhibits or neutralizes the function of a plasma kallikrein protein, e.g., human plasma kallikrein. As used herein, the term “neutralize” means that the activity or function of the protein is inhibited. The inhibition can be complete or partial. In some embodiments, the activity or function of the protein is inhibited at least 10, 20, 30, 40, 50, 60, 70, 80, 90, 95, or 99%. The percent inhibition can be based upon the function or activity of the protein in the absence of the antibody. In some embodiments, the antibody inhibits the clotting function facilitated by plasma kallikrein. Accordingly, the invention provides methods of modulating plasma kallikrein activity, such as contacting plasma kallikrein with a plasma kallikrein antibody as disclosed herein, or a pharmaceutical composition comprising that antibody, to modulate the activity of the plasma kallikrein, such as human plasma kallikrein. In some embodiments, the method comprises administering to a subject the antibodies as provided for herein, or a pharmaceutical composition comprising the same, to modulate plasma kallikrein activity in the subject.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise. The expression “and/or” in connection with three or more recited objects should be understood to have the same meaning unless otherwise understood from the context.

It should be understood that the expression “at least one of” includes individually each of the recited objects after the expression and the various combinations of two or more of the recited objects unless otherwise understood from the context and use.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±5% variation from the nominal value unless otherwise indicated or inferred from the context.

The term “in combination with” as used herein means that the described agents can be administered to an animal or subject together in a mixture, concurrently as single agents or sequentially as single agents in any order.

Where a molecular weight is provided and not an absolute value, for example, of a polymer, then the molecular weight should be understood to be an average molecule weight, unless otherwise stated or understood from the context.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

It should be understood that the order of steps or order for performing certain actions is immaterial so long as the present invention remain operable. Moreover, two or more steps or actions may be conducted simultaneously.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The subject matter is now described with reference to the following examples. These examples are provided for the purpose of illustration only and the claims should in no way be construed as being limited to these examples, but rather should be construed to encompass any and all variations which become evident as a result of the teaching provided herein. Those of skill in the art will readily recognize a variety of non-critical parameters that could be changed or modified to yield essentially similar results.

### EXAMPLES

### Example 1: Generation of Novel Plasma Kallikrein Antibodies

Five prekallikrein (prekal) knock-out mice were immunized via the hock route with active human plasma kallikrein (pkal, Enzyme Research Labs, South Bend, IN). When serum titers indicated a robust immune response, draining lymph nodes were harvested, and lymphocytes were fused with a myeloma line using electrofusion. Resulting hybridomas were plated in thirty 384-well plates, and screening by ELISA for binding to human pkal and prekal. Hybridoma lines with preferential binding to pkal were subcloned and expanded.

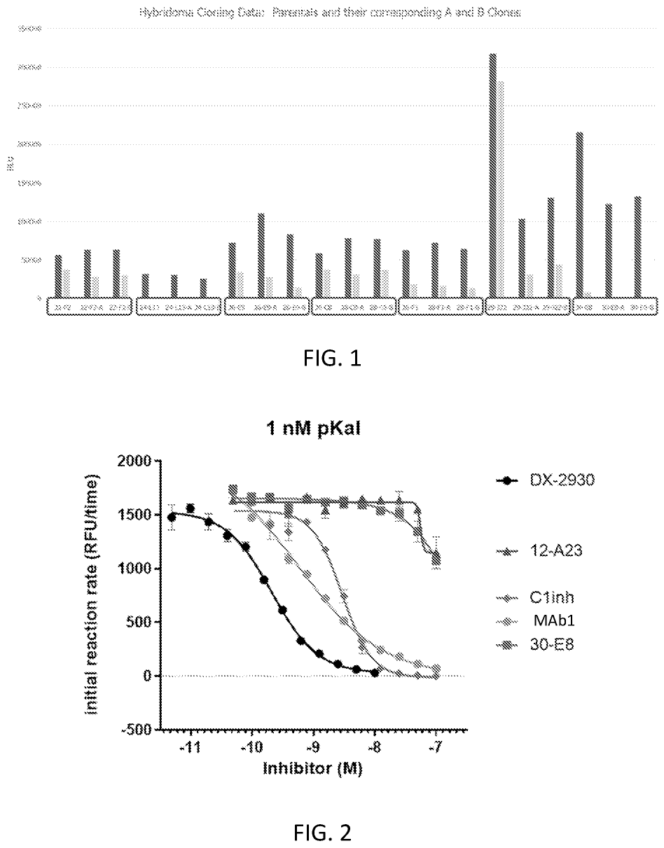

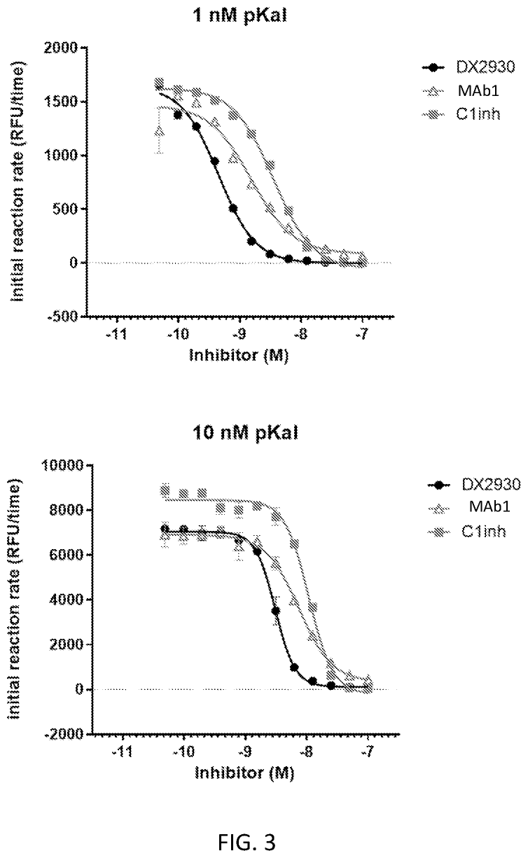

Monoclonal IgGs were retested for pkal and prekal binding by ELISA, and were screened for inhibition of pkal (1 nM enzyme) enzymatic activity in a fluorogenic peptide substrate assay (Kenniston et al. J Biol Chem. 2014 Aug. 22; 289(34):23596-608). As shown in FIG. 1, three monoclonal lineages, including 24L 13, were confirmed to be selective for pkal. FIG. 2 shows that one lineage (24L13, antibody named MAb1) was also inhibitory in the enzyme assay (Kenniston, 2014) compared to a positive control, Clinh, and another antibody, DX-2930, which is described in U.S. Pat. No. 8,816,055. This hybridoma was sequenced to determine heavy and light chain variable domains (SEQ ID NO: 1 and SEQ ID NO: 2; MAb1). As shown in FIG. 3, recombinantly expressed MAb1 retained the inhibitory activity of hybridoma derived IgG. The nucleic acid sequence of the heavy chain variable domain of MAb1 is provided as SEQ ID NO: 33 and the nucleic acid sequence of the light chain variable domain of MAb1 is provided as SEQ ID NO: 34.

Nearest human germline sequences for the murine variable domains were identified by comparison with human germline sequences listed at IMGT (imgt.org) Nested sets of framework modifications were designed for both the heavy and light chains to evolve the murine sequences towards human germlines, pairwise expressed, and tested for binding affinity to pkal, and potency of inhibition of pkal enzymatic activity. Variant VH5_VL4 (MAb2) maintained both binding affinity and inhibitory potency, and was optimally humanized (SEQ ID NO: 3, SEQ ID NO: 4). In addition, an NG structural liability in heavy chain CDR2 was successfully modified to NA to reduce the risk of deamidation (SEQ ID NO: 5).

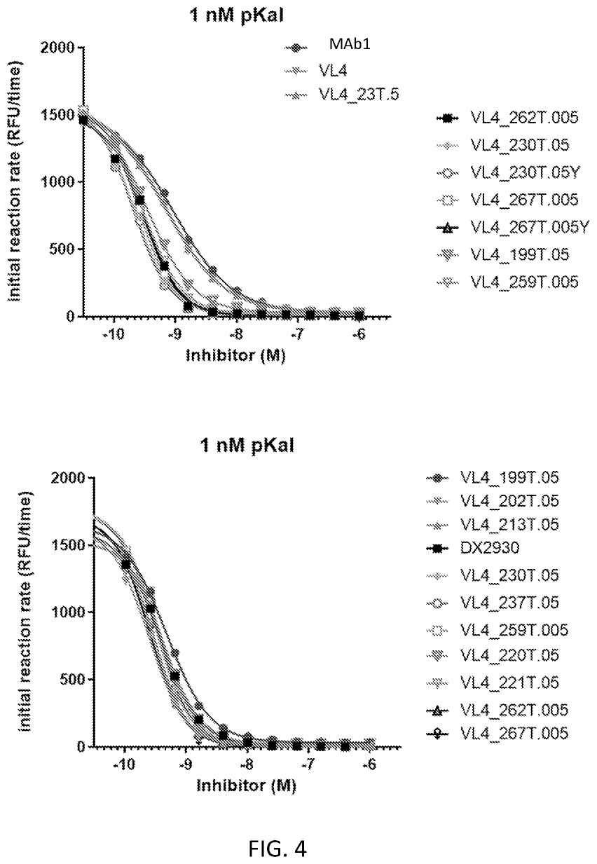

MAb1 light chain CDR sequences were diversified and a Fab phage display library was constructed. Successive selections with pkal and deselections with prekal were used to enrich for pkal selective clones. Selective clones were expressed as IgG and tested for inhibition of pkal enzymatic activity (FIG. 4). Light chain CDR2 variant clone VL4_213T.05 (MAb3-VL; SEQ ID NO: 6) was identified as a selective variant with high potency of inhibition of pkal. This light chain CDR2, grafted into the humanized VL4 variant (SEQ ID NO: 6, MAb3-VL), maintained selectivity and activity when expressed with the humanized, liability fixed heavy chain variable region (SEQ ID NO: 5, MAb3-VH). The humanized and optimized construct of the MAb3 VL and VH sequences was named MAb4 (heavy chain SEQ ID NO: 7, light chain SEQ ID NO: 8) when expressed as full length human IgG1 kappa format.

To further optimize the therapeutic potential of MAb4, the heavy chain constant domains were modified by introducing LS (corresponding to M428L and N434S according to EU numbering) or YTE mutations (corresponding to M252Y, S254T, and T256E according to EU numbering) (SEQ ID 9 and SEQ ID 10, respectively). When expressed with light chain SEQ ID NO: 8, these humanized IgG constructs were designated as MAb4-LS and MAb4-YTE, respectively.

### Example 2: In Vitro Characterization of Plasma Kallikrein Antibodies

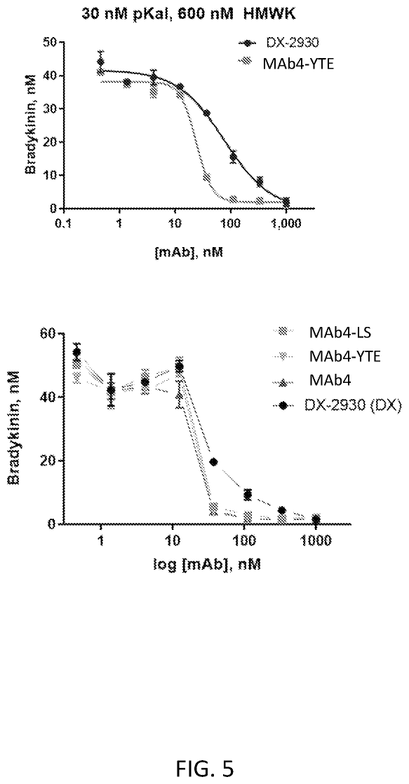

The in vitro potency of MAb4 and its Fc variants, MAb4-YTE and MAb4-LS, as compared to DX-2930, a commercially available monoclonal antibody known to inhibit plasma kallikrein and the subject of clinical studies for the treatment of HAE, was tested in a physiologically relevant functional assay. Specifically, the ability of each antibody to inhibit pkal cleavage of its natural substrate, High Molecular Weight Kininogen (HMWK) was observed. In these experiments 10 or 30 nM pkal was preincubated with inhibitory antibodies for 1 hr at RT, then 600 nM HMWK was added and incubated for 20 minutes and the reaction was quenched by adding 0.5 μM AEBSF. The concentration of bradykinin (BK) released from HMWK as catalyzed by pKal was measured using 1/100 dilution of the reaction product in a commercial ELISA kit (Enzo, Farmingdale, NY). A comparison of the newly generated antibodies and the reference antibody DX-2930 in a pKal inhibition assay is shown in FIG. 5. Mab4 and the Mab4-YTE and Mab4-LS constructs showed more potent inhibition of pKal and steeper Hill slopes as compared to DX-2930. In this context, MAb4 and its Fc variants are potent inhibitors of HMWK cleavage by pkal, and exhibit a Hill slope ˜2 (see, e.g., FIG. 5), consistent with the hypothesis that MAb4 and its Fc variants may be allosteric inhibitors of pkal. Furthermore, the introduction of the YTE or LS modifications into the Mab4 construct did not materially impact the potency of the constructs in the inhibition assay.

The HMWK assay parameters were physiologically relevant as the concentration of high molecular weight kininogen used is the concentration that circulates in humans and the concentration of plasma kallikrein used is in the range of what has been estimated in plasma from patients with hereditary angioedema (HAE) during an attack. Both Mab4-YTE and DX-2930 showed a dose-dependent inhibition of bradykinin production, indicating reduced plasma kallikrein activity and Mab4-YTE exhibited a greater potency for inhibition of plasma kallikrein activity and bradykinin release than DX-2930, as shown in FIG. 5.

The IC₉₀ values for the antibodies were also determined to provide an index of relative potency. In HAE, the therapeutically relevant level of inhibition to prevent HAE attacks is thought to be approximately 90% inhibition of plasma kallikrein. This corresponds to a measurement referred to as IC90, which is the concentration of an agent that results in 90% inhibitory effect. In the HMWK functional assay, as shown in FIG. 5, the IC₉₀ value is the concentration of antibody that results in 90% inhibition of kallikrein activity and is believed to be the therapeutically relevant level of inhibition. The IC₉₀ value for DX-2930 is 300 nM, whereas the IC₉₀ values for MAb4 and its Fc variants is 30 nM. Therefore, this in vitro HMWK functional assay showed that MAb4-YTE had approximately 10-fold higher potency than DX-2930. The results of this assay are consistent with observed clinical efficacy for DX-2930, as clinical trials with DX-2930 have shown that steady state plasma levels of DX-2930 of 200-300 nM are required to optimally reduce the HAE attack rate and maximize attack-free duration.

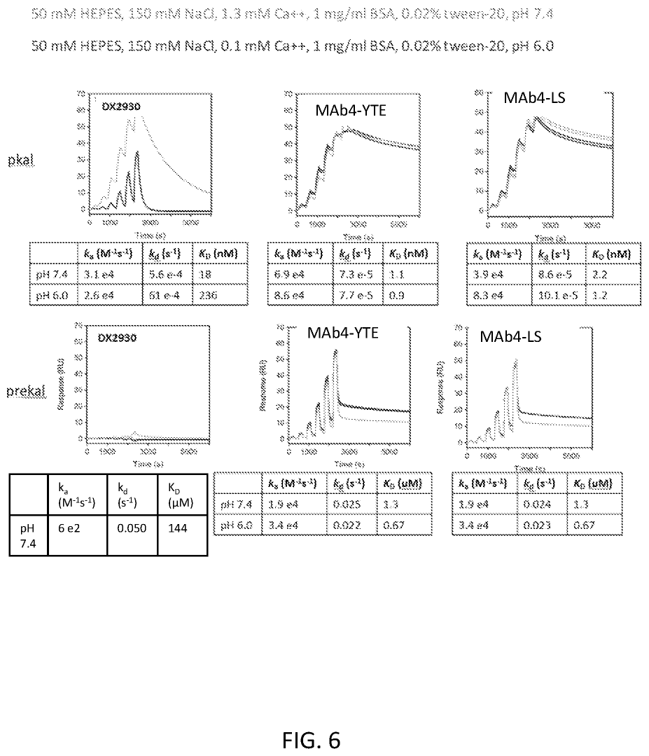

Kinetic properties and selectivity of lead pkal antibodies were examined by SPR at 37° C. and either pH 7.4 or pH 6.0. MAb4-LS (SEQ ID NO: 9 & 8) and MAb4-YTE (SEQ ID NO: 10 & 8) exhibited low single digit nanomolar affinity for pkal, with substantial selectivity over prekal (FIG. 6). In fact, MAb4-LS and MAb4-YTE, as well as DX-2930, have greater than 1000 fold higher affinity for plasma kallikrein than prekallikrein. (FIG. 6) The K_{D} for MAb4-YTE with respect to human plasma kallikrein was 1.1 nM at pH 7.4 and 0.9 nM at pH 6.0, while the K_{D} for MAb4-LS was 2.2 nM at pH 7.4 and 1.2 nM at pH 6.0. These antibodies did not demonstrate any significant pH dependence in target binding to human plasma kallikrein, in particular within the range of pH 6.0 to pH 7.4. In contrast, reference antibody DX-2930 demonstrated a K_{D} of 18 nM at pH 7.4 and 236 nM at pH 6.0 (FIG. 6). As a result, the Mab-YTE and Mab4-LS antibodies have a stronger binding affinity for plasma kallikrein (greater than 5 fold, e.g., 8-16 fold) than the reference antibody DX-2930, consistent with the approximately 10-fold greater potency of Mab4-YTE as compared to DX-2930 in the functional plasma kallikrein inhibition assay. Furthermore, the DX-2930 antibody showed considerable pH sensitivity under the same conditions. For example, the DX-2930 antibody displayed a greater than 10-fold reduction in K_{D} when measured at pH 6 versus pH 7.4. Table 7 shows the Kₐ, K_{d} and K_{D} values for MAb4-YTE and DX-2930 based on SPR binding as described above at pH 7.4, 37° C.

TABLE 7

| | Kₐ (1/Ms) | K_{d} (1/s) | K_{D} (nM) |
|---|---|---|---|
| MAb4-YTE | 6.9 × 10⁴ | 7.3 × 10⁻⁵ | 1.1 |
| DX-2930 | 3.1 × 10⁴ | 5.6 × 10⁻⁴ | 18 |

Collectively, the data presented in FIG. 6 shows that the antibodies Mab4-YTE or Mab4-LS have one of more of the following features:
(a) a K_{D} that is substantially pH independent between pH 6.0-7.4 (e.g., as determined by SPR at 37° C. in a buffer containing 50 mM HEPES, 150 mM NaCl, 0.1 or 1.3 mM Ca²⁺, 1 mg/mL BSA, 0.02% Tween-20)
(b) a K_{D} of between 0.1 nM and 5 nM for human plasma kallikrein (e.g., as determined by SPR at 37° C. in a buffer containing 50 mM HEPES, 150 mM NaCl, 0.1 or 1.3 mM Ca²⁺, 1 mg/mL BSA, 0.02% Tween-20, pH 6.0 or 7.4); and
(c) a K_{D} of between 0.1 nM and 5 nM for human plasma kallikrein and a K_{D} of between 250 nM and 2,000 nM for human prekallikrein (e.g., as determined by SPR at 37° C. in a buffer containing 50 mM HEPES, 150 mM NaCl, 0.1 or 1.3 mM Ca²⁺, 1 mg/mL BSA, 0.02% Tween-20, pH 6.0 or 7.4.

hFcRn binding of the plasma kallikrein antibodies with the Fc mutations (MAb4-LS and MAb4-YTE) was also assessed. Two dilution series of MAb4-LS and MAb4-YTE were made: 2000 nM to 31.25 nM MAb4-LS and MAb4-YTE at pH 6.0 and 2000 nM to 500 nM at pH 7.4 in PBS with 0.05% P20 (polysorbate 20). IgG1 was used as a control antibody. The antibody samples were assessed for FcRn binding via SPR using a Biacore T200. The Biacore chip was a CMS coupled with human FcRn (Sino Biological, Cat. No. CT009-H08H). Running buffer used was PBS 0.05% P20 and pH 6 or pH 7.4. The hFcRn binding at pH 6.0 is shown in Table 8. Both MAb4-LS and MAb4-YTE had increased pH-dependent hFcRn binding due to reduced off rates at pH 6.0 (10 fold slower) as compared to unmodified MAb4 (3.10×10² and 2.77×10² K_{d} as compared to 2.29×10¹ K_{d}) and as compared to DX-2930 (1.67×10¹ K_{d}). At pH 7.4, there was no evidence of binding for all three of the antibodies, suggesting that FcRn binding of MAb4, MAb4-LS and MAb4-YTE is pH dependent [data not shown]. Thus, without wishing to be bound by theory, both the MAb4-LS and MAb4-YTE antibodies may bind better to the FcRn in the endosome (pH 6) and weaker to the FcRn in the blood (pH 7.4) as compared to the parental MAb4 antibody, resulting in antibody recycling back into the blood via the IgG-FcRn recycling pathway, thereby increasing the circulating half-life of MAb4-LS and MAb4-YTE by approximately 3 to 4 fold (see Table 10 in Example 3) over MAb4 and DX-2930.

TABLE 8
hFcRn Binding at pH 6.0

| | Kₐ (1/Ms) | K_{d} (1/s) | K_{D} (M) |
|---|---|---|---|
| MAb4 | 2.7 × 10⁵ | 2.29 × 10⁻¹ | 8.48 × 10⁻⁷ |
| MAb4-LS | 2.96 × 10⁵ | 3.10 × 10⁻² | 1.05 × 10⁻⁷ |
| MAb4-YTE | 1.84 × 10⁵ | 2.77 × 10⁻² | 1.50 × 10⁻⁷ |
| DX-2930 | 8.66 × 10⁵ | 1.67 × 10⁻¹ | 1.93 × 10⁻⁷ |

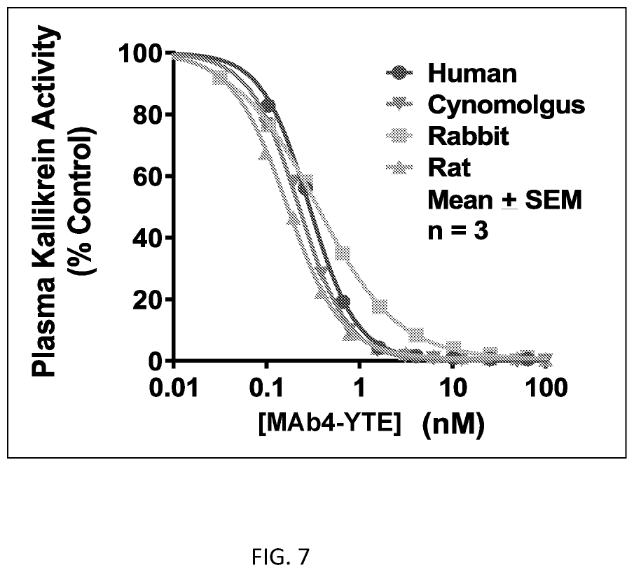

MAb4-YTE was also shown to potently inhibit not only human, but also cynomolgus monkey, rat and rabbit plasma kallikrein enzymatic activity as assessed using a small fluorogenic substrate of pKal, Pro-Phe-Arg-AMC (PFR-AMC). The MAb4-YTE antibody was incubated at the indicated concentrations as shown in the graph in FIG. 7 along with 1 nM pKal from each species for 1 hour at 37° C. in triplicate wells. 10 μM of the fluorogenic reporter substrate PFR-AMC was added and pKal activity was measured in a fluorescent plate reader in kinetic mode for 60 minutes. Linear Regression analyses on the first 40 min of substrate release data from each reaction well were performed to obtain the initial reaction rate (RFU/min) for each inhibitor concentration. The initial reaction rates were fitted (4-parameter fit) to obtain IC₅₀ values, and normalized to obtain percent enzyme activity within the upper and lower bounds of the fitted curve. The IC₅₀ data is shown in Table 9. The level of pKal activity plotted against the concentration of MAb4-YTE in nM is shown in FIG. 7. This data shows that MAb4-YTE is a potent inhibitor of not only human, but also monkey, rabbit, and rat pKal.

TABLE 9
IC₅₀ Values for MAb4-YTE

| | Human | Monkey | Rabbit | Rat |
|---|---|---|---|---|
| IC₅₀ (pM) | 290 | 223 | 352 | 167 |

### Example 3: In Vivo Pharmacokinetic Characterization of Plasma Kallikrein Antibodies

MAb4 (SEQ ID NO: 7 &8), MAb4-LS (SEQ ID NO: 9 & 8) and MAb4-YTE (SEQ ID NO: 10 & 8) were further characterized in cynomolgus pharmacokinetic studies, alongside another plasma kallikrein antibody, DX-2930. These studies of these antibodies were conducted concurrently, but were independent studies rather than a head-to-head comparison. Cynomolgus monkeys, LM-naïve or naive, 3.0-4.0 kg, male, N=18, were purchased from Hainan Jingang Biotech Co. LTD. The antibodies were prepared in 20 mM Histidine, 150 mM NaCl, 0.01% Tween 80, pH 6.5. In these studies, all antibodies were administered to male cynomolgus monkeys (N=3) via IV infusion over 10 min at 5 mg/kg. Blood was collected from cephalic and saphenous veins into pre-cooled citrate acid tubes at each time point. Samples were taken once a week for 42 days (DX-2930 and MAb4) or 84 days (MAb4-YTE and MAb4-LS). Blood samples were put on wet ice and centrifuged at 4° C. to obtain plasma within 15 minutes of sample collection. Plasma samples were stored at approximately −70 C. Presence of each anti-plasma kallikrein antibody in the plasma samples was determined via ELISA using a plate coated with anti-human antibody as the capture agent and human IgG-heavy and light chain monkey-adsorbed antibody as the detection reagent (Southern Biotech, Cat #: 2049-01 and Bethyl, Cat #A80-319P). ELISA plates were read using a SpectraMax M2.

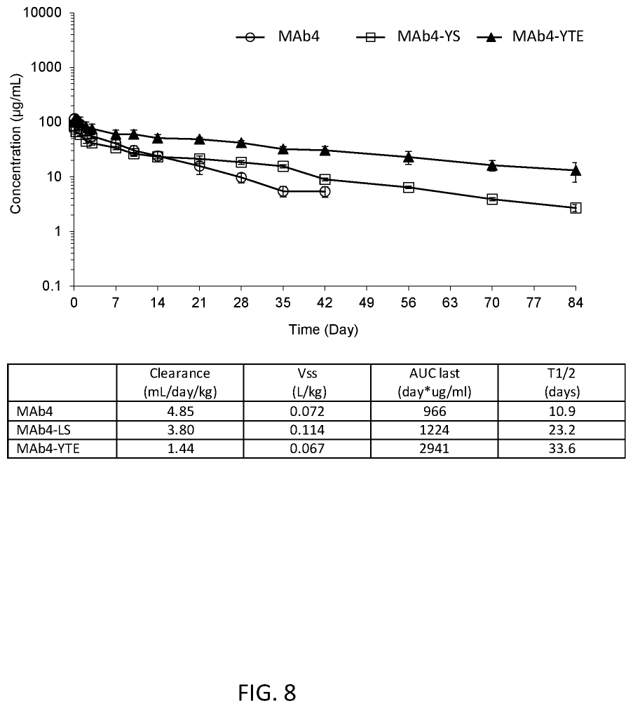

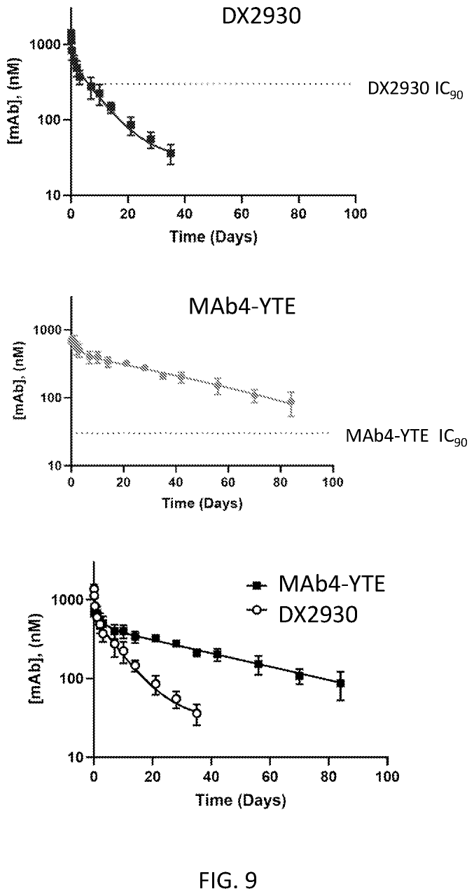

MAb4 and DX-2930 (MAb4 half-life shown in FIG. 8, DX-2930 half-life shown in FIG. 9) had terminal half-lives of 10-12 days, which is consistent with what has been reported in USFDA review documents and publications for DX-2930 in non-human primates. In contrast, MAb4-LS exhibited a terminal half-life of approximately 23 days and MAb4-YTE exhibited a terminal half-life of approximately 34 days (see, FIG. 8). The terminal half-life of MAb4-YTE is about 3 to 4-fold longer than that observed for DX-2930 and MAb4. These results demonstrate the superiority of the MAb4 antibodies with an extended half-life. This result was surprising because it was discovered that the introduction of the LS or YTE mutations into another plasma kallikrein antibody did not increase the half-life (data not shown). In addition, the increased inhibitory potency of the newly generated antibodies and the increased half-life indicate that effective amounts of MAb4-YTE or MAb4-LS may be maintained in vivo for longer periods compared to DX-2930. FIG. 9 shows the PK half-life of MAb4-YTE (middle panel) and DX-2930 (top panel) compared to each antibody's pKal IC90. (The data in the top panel and middle panel are superimposed on each other in the bottom panel to show both MAb4-YTE and DX-2930's plasma levels). DX-2930's plasma levels fall below predicted minimum therapeutic concentration (IC90) by approximately day 10, while MAb4-YTE remains above predicted minimum therapeutic concentration (IC90) for >84 days. Table 10 provides a summary of the PK data from the cynomolgus monkey studies. Without wishing to be bound by theory, the increased pH-dependent FcRn binding of the Fc modified antibodies described in Example 2 resulted in slower clearance and extended half-life in the cynomolgus monkeys.

TABLE 10
Cynomolgus PK

| | Vss (mL/kg) | CI (mL/day/kg) | T ½ (days) |
|---|---|---|---|
| | (Mean ± SD) | (Mean ± SD) | (Mean ± SD) |
| MAb4 | 72.2 ± 11.1 | 4.85 ± 0.87 | 10.9 ± 0.4 |
| MAb4-LS | 114.4 ± 9.6 | 3.80 ± 0.13 | 23.2 ± 3.5 |
| MAb4-YTE | 66.8 ± 6.1 | 1.44 ± 0.33 | 33.6 ± 8.3 |
| DX-2930 | 64.9 ± 15.5 | 5.09 ± 1.18 | 10.5 ± 1.6 |

An evaluation of the in vitro potency data and half-life results from the non-human primate studies suggests the potential duration of efficacy for the antibodies. Specifically, the measured plasma concentration of an antibody at each timepoint from the cynomolgus monkey studies can be used to predict an expected level of plasma kallikrein inhibition at that time point based on the plasma kallikrein inhibition observed for that concentration of antibody in the in vitro potency assay.

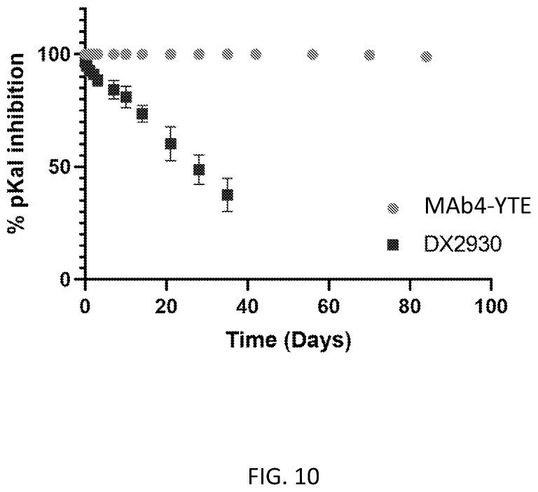

FIG. 10 provides a prophetic model based on plasma concentrations from cynomolgus PK studies and pKal inhibition determined in in vitro functional assay comparing the percent pKal inhibition in vivo with Mab4-YTE and DX-2930 based on the HMWK assay data and the in vivo PK data. In the cynomolgus monkey studies, DX-2930 plasma levels fell below the minim therapeutic concentration, or IC90, predicted by the in vitro potency assay by approximately day 10, and by day 20 were at levels that the in vitro potency assay predicted would result in approximately 50% inhibition of plasma kallikrein. In contrast, in the cynomolgus monkey studies, MAb4-YTE plasma levels remained above the IC90 predicted by the in vitro potency assay for 84 days, which was the full duration of the experiment.

These preclinical data suggest that at equal doses, MAb4-YTE would have a half-life in humans of several months and thus a significantly longer duration of action than DX-2930. This could potentially enable a lower dose of MAb4-YTE that would have a longer duration of action than DX-2930. Accordingly, MAb4-YTE could be an effective prophylactic therapy for patients with HAE by inhibiting the pathological activity of plasma kallikrein for an extended time period. In addition, the antibody could be dosed infrequently while maintaining therapeutically effective plasma levels necessary to prevent HAE attacks. It is possible that an antibody such as MAb4-YTE or MAb4-LS could therefore be administered at infrequent intervals, for example, every three months, or at even longer intervals. This would be a significant advantage over currently available therapies for HAE such DX-2930. MAb4-YTE is therefore likely to combine the benefits of infrequent dosing, lower dosing, and inhibiting HAE attacks over long periods of time.

### Example 4. In Vitro Characterization of Off-Target Binding of Plasma Kallikrein Antibodies

A membrane proteome array assay was performed to identify any non-specific binding of MAb4. No off target binding was observed for MAb4 (data not shown).

Additionally, the antibodies were tested to determine if they contained effector function. The antibodies were found not to have effector function (data not shown).

The specificity of the antibodies was also determined by evaluating whether the antibodies bound to unrelated serine proteases. The antibodies were screened against a panel of serine proteases (˜20) and no significant cross-reactivity binding was observed (data not shown).

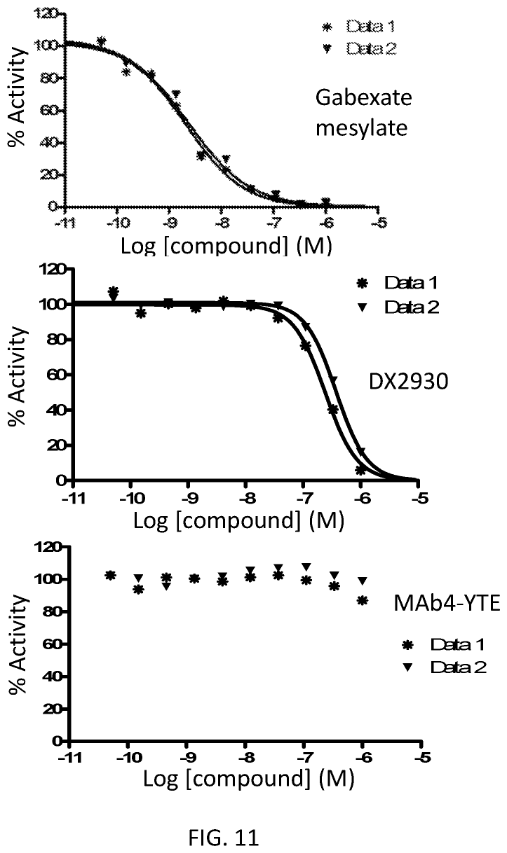

A trypsin inhibition assay was developed and performed to determine if MAb4 exhibited any cross-reactivity. 0.25 nM trypsin, human pancrease (Cat #16-19-032000, Athens Research and Technology Inc) and a dilution series of MAb4-YTE starting at 1 μM were incubated together for 2 hours with PFR-AMC (fluorogenic substrate, Bachem, cat #4004023) in buffer. Fluorescence intensity was measured every 5 minutes. Gabexate mesylate was used as a positive control, in a dilution series starting at 1 μM. The assay was performed in duplicate. The results are provided in FIG. 11 and Table 11. MAb4-YTE did not inhibit trypsin activity and no IC50 could be calculated. In contrast, antibody DX-2930 inhibited the activity of trypsin with an IC50 (243 nM to 376 nM) that was within DX-2930's potency range for inhibiting the functional activity of plasma kallikrein (˜300 nM). Thus, Mab4-YTE demonstrated a better protease selectivity profile relative to DX-2930.

TABLE 11

Compound IC50 (M)

| | Data 1 | Data 2 |
|---|---|---|
| Gabexate mesylate (GM) | 2.13E−09 | 2.33E−09 |
| DX-2930 | 2.43E−07 | 3.76E−07 |
| MAb4-YTE | NA | NA |

Additional serine protease inhibition assays were developed and performed to determine if MAb4 exhibited any cross-reactivity. MAb4-YTE and DX-2930 were tested in duplicate in a 10-dose assay with a 3-fold serial dilution starting at 1 μM against 16 proteases. Control compounds were tested in a 10-dose assay with 3-fold serial dilution starting at 10 μM or 100 uM (tPA, Matriptase 2, Kallikrein 5, Kallikrein 2, Kallikrein 1, Granzyme B, and APC). The protease activities were monitored as a time-course measurement of the increase in fluorescence signal from a fluorescently-labeled peptide substrate, and the initial linear portion of the signal slope (signal/min) was analyzed. % Enzyme activity for antibody titrations were calculated respective to vehicle controls in presence of PBS buffer as 100% Activity. IC50 values for proteinase inhibition by MAb4-YTE and DX-2930 are summarized in Table 12 below.

TABLE 12

| | Compound IC50 (M) | | | | Control | Control |
|---|---|---|---|---|---|---|
| | MAb4-YTE | | DX-2930 | | Compound | compound |
| Target: | Duplicate 1 | Duplicate 2 | Duplicate 1 | Duplicate 2 | IC50 (M) | ID |

| | | | | | |
|---|---|---|---|---|---|
| 1 | Activated | | | 6.48E−08 | Gabexate |
| | Protein C | | | | mesylate (GM) |
| 2 | Cathepsin G | | | 4.09E−06 | Chymostatin |
| 3 | Factor VIIa | | | 1.41E−07 | PCI 27483 |
| 4 | Factor XIa | | | 1.79E−07 | GM |
| 5 | Factor XIIa | | | 2.32E−08 | Nafamostat |
| | | | | | mesylate |
| 6 | Granzyme B | | | 1.78E−07 | IETD-CHO |
| 7 | Hepsin | *>1.00E−06 | *>1.00E−06 | 5.10E−08 | GM |
| 8 | Kallikrein 1 | | | 2.99E−06 | Leupeptin |
| 9 | Kallikrein 2 | | | 1.39E−07 | GM |
| 10 | Kallikrein 5 | | | 8.73E−06 | GM |
| 11 | Kallikrein 12 | | | 3.25E−08 | GM |
| 12 | Matriptase 2 | | | 2.01E−07 | GM |
| 13 | Plasmin | | | 2.53E−07 | GM |
| 14 | tPA | | | 1.06E−06 | GM |
| 15 | Thrombin a | *>1.00E−06 | *>1.00E−06 | 8.37E−07 | GM |
| 16 | Urokinase | | | 1.65E−08 | GM |

Empty cells indicate no inhibition or compound activity that could not be fit to an IC50 curve. The IC50 value higher than 1 μM was estimated based on the best curve fitting available.

No IC50 could be calculated for MAb4-YTE against any serine protease tested in this assay. IC50s greater than 1 μM (1.00E-06) were estimated for DX-2930 against Hepsin and Thrombin A. The difference in the serine protease inhibition profile was consistent with a greater protease selectivity for Mab4-YTE as compared to DX-2930.

In sum, MAb4-YTE did not inhibit trypsin activity or any tested serine protease and thus does not exhibit off target inhibitory activity. In contrast, the DX-2930 plasma kallikrein antibody showed significant trypsin inhibitory activity as well as off-target inhibitory activity with respect to Hepsin and Thrombin A, demonstrating the superior properties of MAb4-YTE's specificity and selectivity for its target due to lack of off-target activity.

### Example 5: Epitope Characterization of Plasma Kallikrein Antibodies

### Competition Binding Assay

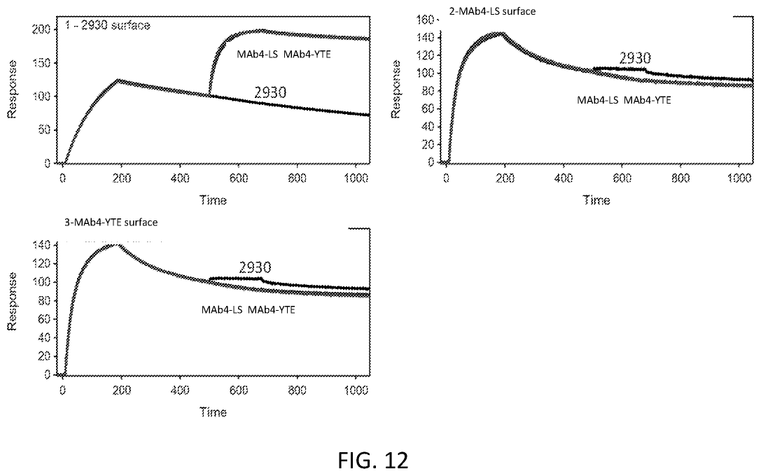

SPR based competition experiments (Biosensor Tools, Salt Lake City, UT) indicate that MAb4 binds to an epitope distinct from that of DX-2930 (FIG. 12), which has been shown to be an active site binding, competitive inhibitor (Kenniston, 2014). DX-2930, MAb4-LS and MAb4-YTE were amine coupled to Xantec 30M sensor chips using standard NHS/ECD coupling with 10 mM NaAc pH 5.0. For the capture and stacking study the running buffer contained HBS with 1.3 mM Ca++ with 1 mg/ml BSA and 0.02% p20 at pH 7.4. Human plasma kallikrein was captured onto the different antibody surfaces (time 0-180 seconds, at 1 μM) and then each mAb was tested over each surface-bound complex at 1 μM.

No binding was observed when each antibody was tested against itself (FIG. 12). However, both MAb4-LS and MAb4-YTE bound to the kallikrein captured by the DX-2930 antibody surface (FIG. 12). In addition, DX-2930 bound to the kallikrein captured by both the MAb4-LS and MAb4-YTE antibodies (FIG. 12). These results demonstrate that MAb4-LS and MAb4-YTE bind to a different epitope than DX-2930.

Cross-Linking and Mass Fingerprinting of pKal-Antibody Complexes

First, human pKal was characterized via peptide mass fingerprinting. Human plasma kallikrein protein was subjected to trypsin, chymotrypsin, Asp-N, elastase and thermolysin proteolysis followed by nLC-QExactive+Orbitrap MS/MS analysis. An nLC Ultimate 3000-RSLC system in line with a QExactive+mass spectrometer (Thermo Scientific) was used. 7 μM human pKal was incubated with 1 μL of DSS d0/d12 (2 mg/mL; DMF) before 180 minutes incubation time at room temperature. A control sample was prepared without addition of the cross-linking regent. Samples were incubated for 180 minutes at room temperature. After incubation, the cross-linking reaction was stopped by adding 1 μL of Ammonium Bicarbonate (20 mM final concentration) and incubated for 60 minutes at room temperature. Then, both tubes were dried using speedvac before 8M urea resuspension (10 μL). After mixing, DTT (1 μL, 500 mM) was added to both tubes. The mixture was then incubated 60 minutes at 37° C. After incubation, iodoacetamide (1 μL, 1M) was added to each tube before additional 60 minutes incubation at room temperature, in a dark room. After incubation, 100 μL of the proteolytic buffer were added to both tubes. Trypsin buffer contained 50 mM Ambic pH 8.5, 5% acetonitrile; chymotrypsin buffer contained Tris HCl 100 mM, CaCL2 10 mM pH 7.8; the ASP-N buffer contained phosphate buffer 50 mM pH 7.8; elastase buffer contained Tris HCl 50 mM pH 8.0 and thermolysin buffer contained Tris HCl 50 mM, CaCL2 0.5 mM pH 9.0.

### Trypsin Proteolysis

100 μL of the reduced/alkyled human pKal were mixed with 1 μL of trypsin (Promega) with the ratio 1/100. The proteolytic mixture was incubated overnight at 37° C.

### Chymotrypsin Proteolysis

100 μL of the reduced/alkyled human pKal were mixed with 0.5 μL of chymotrypsin (Promega) with the ratio 1/200. The proteolytic mixture was incubated overnight at 25° C.

ASP-N Proteolysis

100 μL of the reduced/alkyled human pKal were mixed with 0.5 μL of ASP-N (Promega) with the ratio 1/200. The proteolytic mixture was incubated overnight at 37° C.

### Elastase Proteolysis

100 μL of the reduced/alkyled human pKal were mixed with 1 μL of elastase (Promega) with the ratio 1/100. The proteolytic mixture was incubated overnight at 37° C.

### Thermolysin Proteolysis

100 μL of the reduced/alkyled human pKal were mixed with 2 μL of thermolysin (Promega) with a ratio 1/50. The proteolytic mixture was incubated overnight at 70° C.

After digestion formic acid 1% final was added to the solution. After proteolysis, 1 μL of the peptide solution generated by proteolysis was loaded onto a nano-liquid chromatography system (Ultimate 3000-RSLC). Quadrupole-Orbitrap MS analysis as performed according to manufacturer instructions. Overlap mapping of the trypsin, chymotrypsin, ASP-N, elastase and thermolysin peptides was obtained based on the proteolysis MS results, covering 99.05% of the human pKal sequence.

Next, the epitope of human pKal/MAb4-YTE and human pKal/DX-2930 complexes were determined as described above after incubation with deuterated cross-linkers. After enrichment of the cross-linked peptides, the samples were analyzed by high resolution mass spectrometry (nLC-Quadrupole-Orbitrap MS) and the data generated were analyzed using XQuest and Stavrox software.

20 μL of the human pKal/MAb4-YTE (1.4 μM and 0.8 μM) and human pKal/DX-2930 (1.4 μM and 0.4 μM) mixtures prepared were mixed with 2 μL of DSS d0/d12 (2 mg/mL; DMF) and incubated for 180 minutes at room temperature. After incubation, the reaction was stopped by adding 1 μL of Ammonium Bicarbonate (20 mM final concentration) before 1h incubation at room temperature. Then, the solution was dried using a speedvac before H2O 8M urea suspension (20 μL). After mixing, 2 μL of DTT (500 mM) was added to the solutions. The mixtures were then incubated for 1 hour at 37° C. After incubation, 2 μL of iodoacetamide (1M) were added before 1 hour incubation time at room temperature, in a dark room. After incubation, 80 μL of the proteolytic buffer were added. The trypsin buffer contained 50 mM Ambic pH 8.5, 5% acetonitrile; the Chymotrypsin buffer contained Tris HCl 100 mM, CaCl2) 10 mM pH 7.8; the ASP-N buffer contained phosphate buffer 50MM pH 7.8; the elastase buffer contained Tris HCl 50 mM pH 8.0 and the thermolysin buffer contained Tris HCl 50 mM, CaCl2) 0.5 mM pH 9.0. Proteolysis with trypsin, chymotrypsin, ASP-N, elastase, and Thermolysin was performed using the conditions described above for the pKal protein. The cross-linked peptides were analyzed using Xquest version 2.0 and Stavrox 3.6. software.

The molecular interface between human-PKAL and MAb4-YTE was characterized using chemical cross-linking, High-Mass MALDI mass spectrometry and nLC-Orbitrap mass spectrometry. After trypsin, chymotrypsin, ASP-N, elastase and thermolysin proteolysis of the protein complex human-PKAL/MAb4-YTE with deuterated d0d12, the nLC-orbitrap MS/MS analysis detected ten cross-linked peptides between human-PKAL and MAb4-YTE.

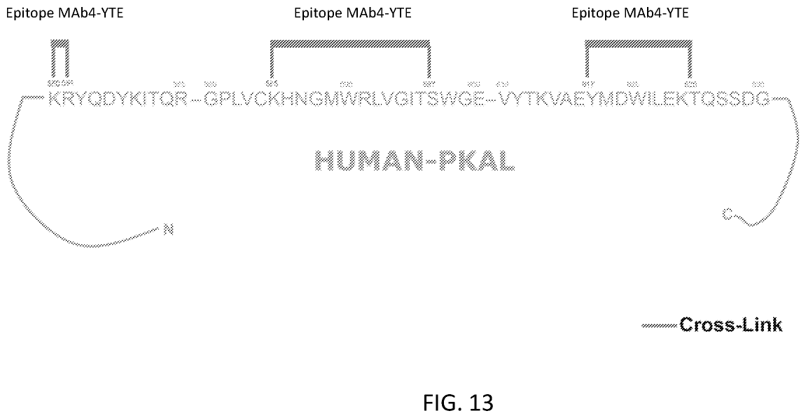

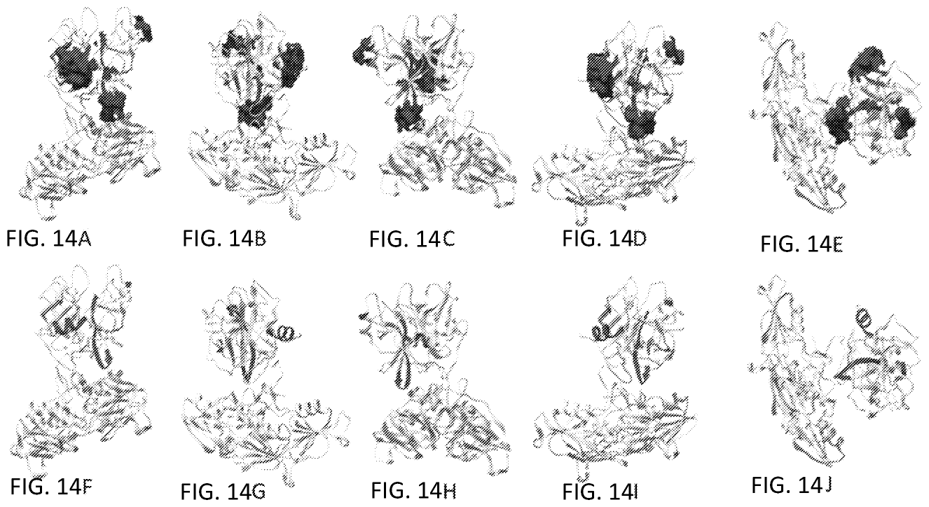

Analysis of the cross-linking data showed that the interaction of MAB4-YTE includes the following amino acids on human pKal: 550, 551, 585, 591, 597, 617, 625. FIG. 13 provides a diagram of the peptide interaction of human pKal and MAB4-YTE. The human pKal sequence is shown on the bottom, and the cross-linked epitopes of Mab4-YTE are indicated at residues 550, 551, 585, 597, 617, and 625. FIG. 14A-J shows a ribbon/surface and a ribbon model of the antibody epitope sites on human pKal. The MAb4-YTE antibody epitope sites were modeled on PDB structure 601G, which used UNIPROT P03952 as the reference sequence. Amino acids indicated in a darker color in the models correspond to residues 550-551 (KR (SEQ ID NO:37)), residues 585-597 (KHNGMWRLVGITS (SEQ ID NO: 38)) and residues 617-625 (YMDWILEKT (SEQ ID NO: 39) of the human pKal sequence.

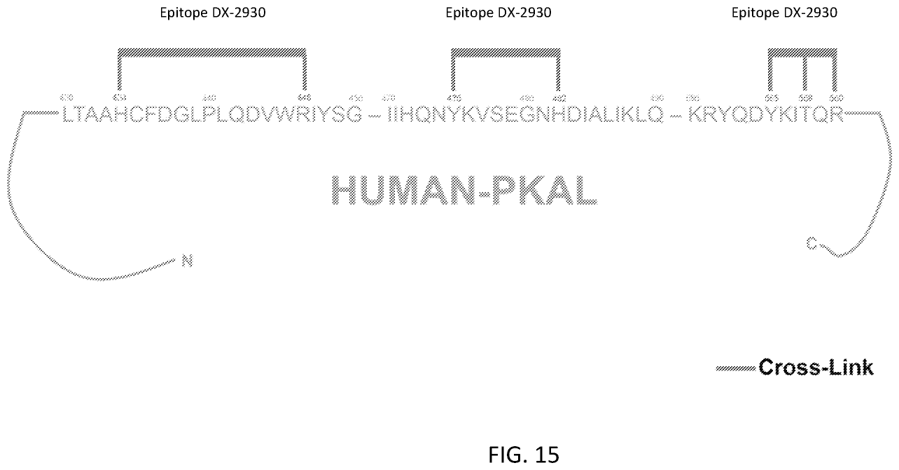

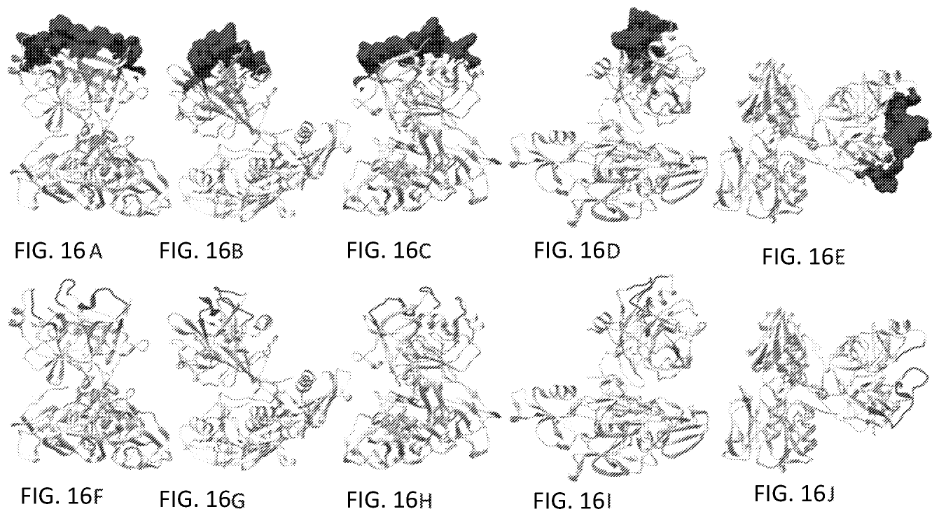

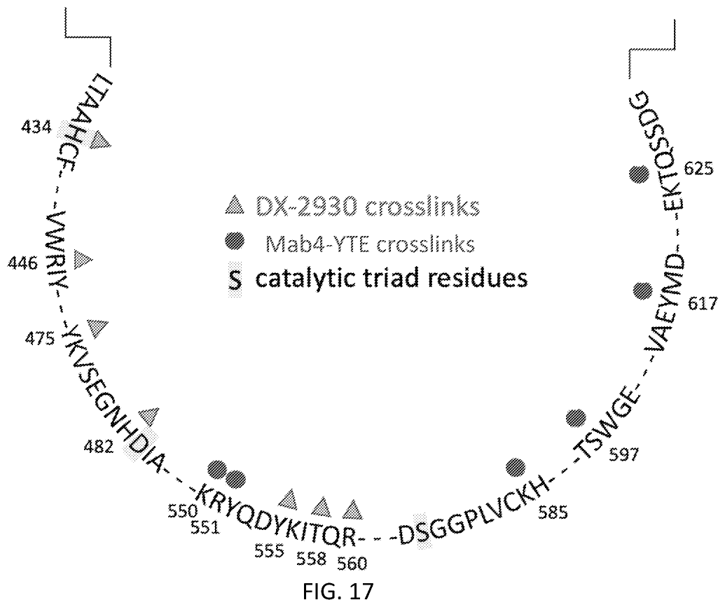

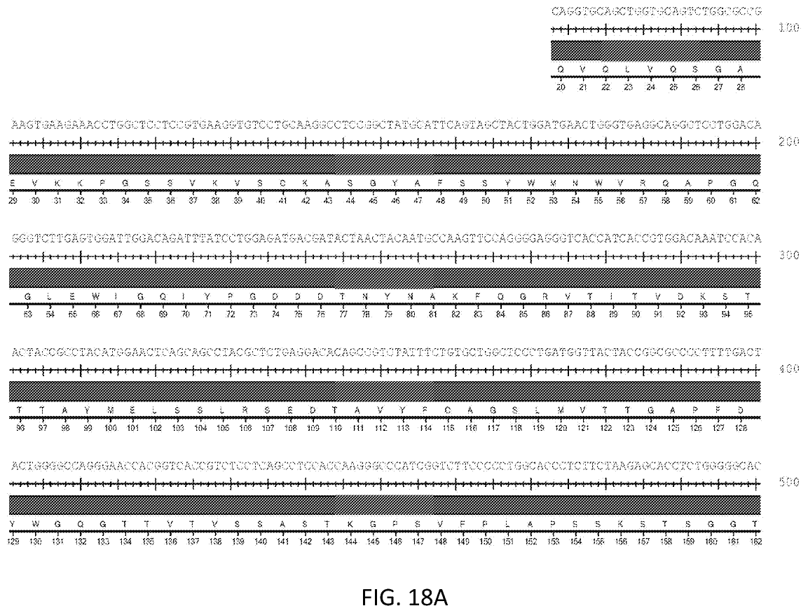

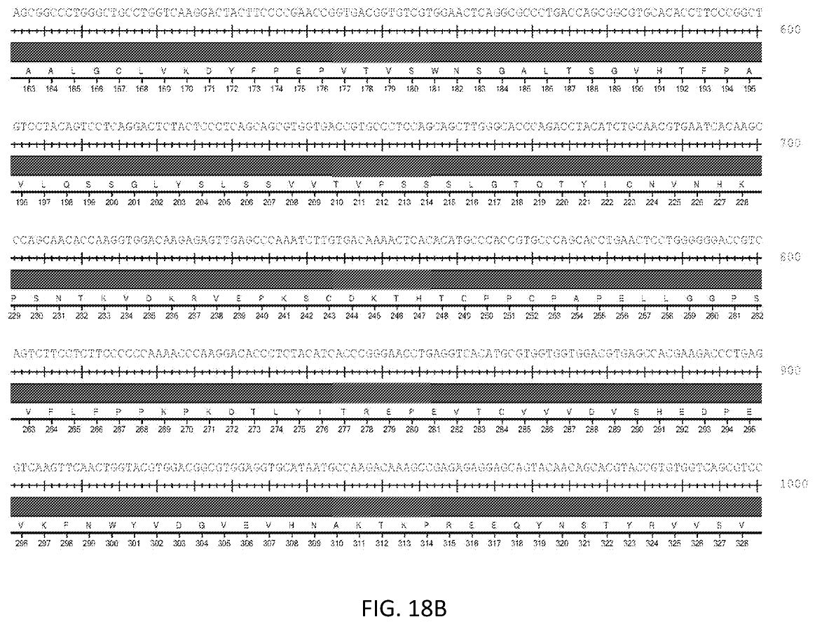

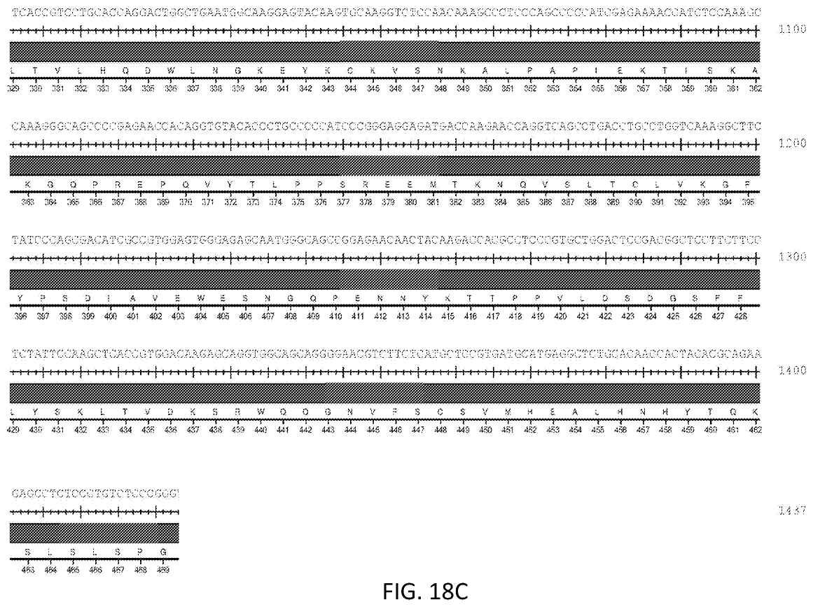

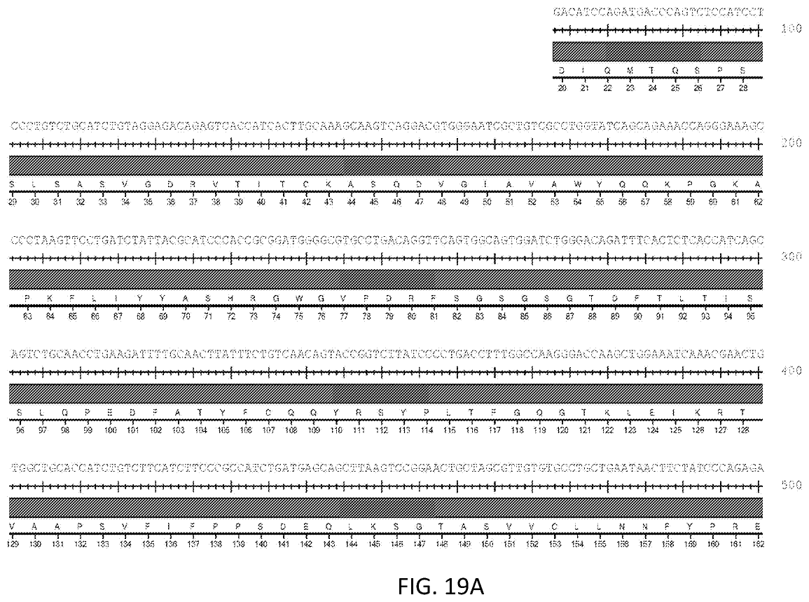

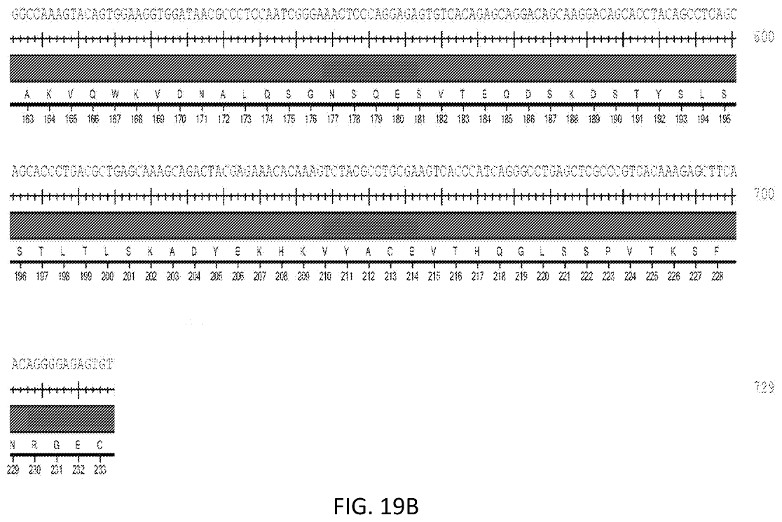

Analysis of the cross-linking data showed that the interaction of DX-2930 includes the following amino acids on human pKal: 434, 446, 475, 482, 555, 558, 560. FIG. 15 provides a diagram of the peptide interaction of human pKal and DX-2930. The human pKal sequence is shown on the bottom, and the cross-linked epitopes of DX-2930 are indicated at residues 434, 446, 475, 482, 555, 558, and 560. FIG. 16A-J shows a ribbon/surface and a ribbon model of the antibody epitope sites on human pKal. The DX-2930 antibody epitope sites were modeled on PDB structure 601G. Amino acids indicated in a darker color in the models correspond to residues 434-446 (HCFDGLPLQDVWR (SEQ ID NO: 40)), residues 475-482 (YKVSEGNH (SEQ ID NO: 41)) and residues 555-560 (YKITQR (SEQ ID NO: 42)) of the human pKal sequence. FIG. 17 provides a map showing the residues of human pKal cross-linked by the antibody DX-2930 as compared to the antibody MAb4-YTE

The crosslinking mass spectrometry data confirms that MAB4-YTE binds to a different epitope on human pKal as compared to DX-2930. Without wishing to be bound by theory, the different binding site on plasma kallikrein for Mab4-YTE as compared with DX-2930 may explain the improved protease selectivity profile, lack of pH sensitivity of plasma kallikrein binding and overall superior profile of Mab4-YTE as compared with DX-2930.

### INCORPORATION BY REFERENCE

The entire disclosure of each of the patent and scientific documents referred to herein is incorporated by reference for all purposes.

### EQUIVALENTS

The present embodiments are not to be limited in scope by the specific embodiments described herein. Indeed, various modifications in addition to those described herein will become apparent to those skilled in the art from the foregoing description and the accompanying figures. Such modifications are intended to fall within the scope of the embodiments and any appended claims.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

SEQUENCES

| SEQ ID | | |
|---|---|---|
| NO: | Description | Sequence |
| 1 | MAb1-VH | QVQLQQSGAELVKPGASVKISCKASGYAFSSYWMNWVKQRPGEGLE |
| | | WIGQIYPGDDDTNYNGKFKGKATLTVDKSSTTAYMQLSSLTSEDSA |
| | | VYFCAGSLMVTTGAPFDYWGQGTTLTVSS |
| 2 | MAb1-VL | DIVMTQSHKFMSTSVGDRVSITCKASQDVGIAVAWYQQKPGQSPKF |
| | | LIYWASTRHTGVPDRFTGSGSGTDFTLTIRNVQSEDLSDYFCQQYR |
| | | SYPLTFGSGTKLELK |
| 3 | MAb2-VH | QVQLVQSGAEVKKPGSSVKVSCKASGYAFSSYWMNWVRQAPGQGLE |
| | | WIGQIYPGDDDTNYNGKFQGRVTITVDKSTTTAYMELSSLRSEDTA |
| | | VYFCAGSLMVTTGAPFDYWGQGTTVTVSS |
| 4 | MAb2-VL | DIQMTQSPSSLSASVGDRVTITCKASQDVGIAVAWYQQKPGKAPKF |
| | | LIYWASTRHTGVPDRFSGSGSGTDFTLTISSLQPEDFATYFCQQYR |
| | | SYPLTFGQGTKLEIK |
| 5 | MAb3-VH | QVQLVQSGAEVKKPGSSVKVSCKASGYAFSSYWMNWVRQAPGQGLE |
| | | WIGQIYPGDDDTNYNAKFQGRVTITVDKSTTTAYMELSSLRSEDTA |
| | | VYFCAGSLMVTTGAPFDYWGQGTTVTVSS |
| 6 | MAb3-VL | DIQMTQSPSSLSASVGDRVTITCKASQDVGIAVAWYQQKPGKAPKF |
| | | LIYYASHRGWGVPDRFSGSGSGTDFTLTISSLQPEDFATYFCQQYR |
| | | SYPLTFGQGTKLEIK |
| 7 | MAb4 heavy chain IgG1 | QVQLVQSGAEVKKPGSSVKVSCKASGYAFSSYWMNWVRQAPGQGLE |
| | format (with constant | WIGQIYPGDDDTNYNAKFQGRVTITVDKSTTTAYMELSSLRSEDTA |
| | domain shown in bold) | VYFCAGSLMVTTGAPFDYWGQGTTVTVSSASTKGPSVFPLAPSSKS |
| | | TSGGTAALGCLVKDYFPEPVTVSWNSGALTSGVHTFPAVLQSSGLY |
| | | SLSSVVTVPSSSLGTQTYICNVNHKPSNTKVDKRVEPKSCDKTHTC |
| | | PPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVS |
| | | LTCLVKGFYPSDIAVEWESNGQPENNYKTTPPVLDSDGSFFLYSKL |
| | | TVDKSRWQQGNVFSCSVMHEALHNHYTQKSLSLSPG |
| 8 | MAb4 Light chain human | DIQMTQSPSSLSASVGDRVTITCKASQDVGIAVAWYQQKPGKAPKF |
| | kappa format (with | LIYYASHRGWGVPDRFSGSGSGTDFTLTISSLQPEDFATYFCQQYR |
| | constant domain shown | SYPLTFGQGTKLEIKRTVAAPSVFIFPPSDEQLKSGTASVVCLLNN |
| | in bold) | FYPREAKVQWKVDNALQSGNSQESVTEQDSKDSTYSLSSTLTLSKA |
| | | DYEKHKVYACEVTHQGLSSPVTKSFNRGEC |
| 9 | MAb4-heavy chain IgG1 | QVQLVQSGAEVKKPGSSVKVSCKASGYAFSSYWMNWVRQAPGQGLE |
| | format with M428L and | WIGQIYPGDDDTNYNAKFQGRVTITVDKSTTTAYMELSSLRSEDTA |
| | a N434S half-life | VYFCAGSLMVTTGAPFDYWGQGTTVTVSSASTKGPSVFPLAPSSKS |
| | extension | TSGGTAALGCLVKDYFPEPVTVSWNSGALTSGVHTFPAVLQSSGLY |
| | substitutions (with | SLSSVVTVPSSSLGTQTYICNVNHKPSNTKVDKRVEPKSCDKTHTC |
| | constant domain shown | PPCPAPELLGGPSVFLFPPKPKDTLMISRTPEVTCVVVDVSHEDPE |
| | in bold) | VKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVS |
| | | LTCLVKGFYPSDIAVEWESNGQPENNYKTTPPVLDSDGSFFLYSKL |
| | | TVDKSRWQQGNVFSCSVLHEALHSHYTQKSLSLSPG |
| 10 | MAb4- heavy chain IgG1 | QVQLVQSGAEVKKPGSSVKVSCKASGYAFSSYWMNWVRQAPGQGLE |
| | format with M252Y, | WIGQIYPGDDDTNYNAKFQGRVTITVDKSTTTAYMELSSLRSEDTA |
| | S254T, and T256E half- | VYFCAGSLMVTTGAPFDYWGQGTTVTVSSASTKGPSVFPLAPSSKS |
| | life extension | TSGGTAALGCLVKDYFPEPVTVSWNSGALTSGVHTFPAVLQSSGLY |
| | substitutions (with | SLSSVVTVPSSSLGTQTYICNVNHKPSNTKVDKRVEPKSCDKTHTC |
| | constant domain shown | PPCPAPELLGGPSVFLFPPKPKDTLYITREPEVTCVVVDVSHEDPE |
| | in bold) | VKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVS |
| | | LTCLVKGFYPSDIAVEWESNGQPENNYKTTPPVLDSDGSFFLYSKL |
| | | TVDKSRWQQGNVFSCSVMHEALHNHYTQKSLSLSPG |
| 11 | LCDR1 MAb1, 2, 3, 4 | KASQDVGIAVA |
| | (Kabat, AbM) | |
| 12 | LCDR2 MAb 1, 2, | WASTRHT |
| | (Kabat, AbM) | |
| 13 | LCDR3 MAb 1, 2, 3, 4 | QQYRSYPLT |
| | (Kabat) | |
| 14 | HCDR1 MAb 1, 2, 3, 4 | SYWMN |
| | (Kabat) | |
| 15 | HCDR2 MAb 1 (Kabat) | QIYPGDDDTNYNGKFKG |
| 16 | HCDR3 MAb 1, 2, 3, 4 | SLMVTTGAPFDY |
| | (Kabat) | |
| 17 | HCDR2 MAb 2 (Kabat) | QIYPGDDDTNYNGKFQG |
| 18 | LCDR2 MAb 3, 4 | YASHRGW |
| | (Kabat, AbM) | |
| 19 | HCDR2 Mab 3, 4 (Kabat) | QIYPGDDDTNYNAKFQG |
| 20 | LCDR1 MAb1, 2, 3, 4 | SQDVGIA |
| | (Chothia) | |
| 21 | LCDR2 MAb 1, 2 | WAS |
| | (Chothia, IMGT) | |
| 22 | LCDR3 MAb 1, 2, 3, 4 | YRSYPLT |
| | (Chothia) | |
| 23 | HCDR1 MAb 1, 2, 3, 4 | GYAFSSY |
| | (Chothia) | |
| 24 | HCDR2 MAb 1, 2, 3, 4 | YPGDDD |
| | (Chothia) | |
| 25 | HCDR3 MAb 1, 2, 3, 4 | SLMVTTGAPFDY |
| | (Chothia, AbM) | |
| 26 | LCDR2 MAb 3, 4 | YAS |
| | (Chothia, IMGT) | |
| 27 | LCDR1 MAb 1, 2, 3, 4 | QDVGIA |
| | (IMGT) | |
| 28 | LCDR3 MAb 1, 2, 3, 4 | QQYRSYPLT |
| | (IMGT, AbM) | |
| 29 | HCDR1 MAb 1, 2, 3, 4 | GYAFSSYW |
| | (IMGT) | |
| 30 | HCDR2 MAb 1, 2, 3, 4 | IYPGDDDT |
| | (IMGT) | |
| 31 | HCDR 3 MAb 1, 2, 3, 4 | AGSLMVTTGAPFDY |
| | (IMGT) | |
| 32 | Sequence of human | MILFKQATYFISLFATVSCGCLTQLYENAFFRGGDVASMYTPNAQY |
| | plasma kallikrein | CQMRCTFHPRCLLFSFLPASSINDMEKRFGCFLKDSVTGTLPKVHR |
| | (Uniprot P03952) | TGAVSGHSLKQCGHQISACHRDIYKGVDMRGVNFNVSKVSSVEECQ |
| | | KRCTNNIRCQFFSYATQTFHKAEYRNNCLLKYSPGGTPTAIKVLSN |
| | | VESGFSLKPCALSEIGCHMNIFQHLAFSDVDVARVLTPDAFVCRTI |
| | | CTYHPNCLFFTFYTNVWKIESQRNVCLLKTSESGTPSSSTPQENTI |
| | | SGYSLLTCKRTLPEPCHSKIYPGVDFGGEELNVTFVKGVNVCQETC |
| | | TKMIRCQFFTYSLLPEDCKEEKCKCFLRLSMDGSPTRIAYGTQGSS |
| | | GYSLRLCNTGDNSVCTTKTSTRIVGGTNSSWGEWPWQVSLQVKLTA |
| | | QRHLCGGSLIGHQWVLTAAHCFDGLPLQDVWRIYSGILNLSDITKD |
| | | TPFSQIKEIIIHQNYKVSEGNHDIALIKLQAPLNYTEFQKPICLPS |
| | | KGDTSTIYTNCWVTGWGFSKEKGEIQNILQKVNIPLVTNEECQKRY |
| | | QDYKITQRMVCAGYKEGGKDACKGDSGGPLVCKINGMWRLVGITSW |
| | | GEGCARREQPGVYTKVAEYMDWILEKTQSSDGKAQMQSPA |
| 33 | Nucleic acid sequence | CAGGTTCAGCTGCAGCAGTCTGGGGCTGAGCTGGTGAAGCCTGGGG |
| | of heavy chain | CCTCAGTGAAGATTTCCTGCAAAGCTTCTGGCTACGCATTCAGTAG |
| | variable domain of | CTACTGGATGAACTGGGTGAAGCAGAGGCCTGGAGAGGGTCTTGAG |
| | MAb1 | TGGATTGGACAGATTTATCCTGGAGATGATGATACTAACTACAACG |
| | | GAAAGTTCAAGGGCAAGGCCACACTGACTGTAGACAAATCCTCCAC |
| | | CACAGCCTACATGCAGCTCAGCAGCCTGACCTCTGAGGACTCTGCG |
| | | GTCTATTTCTGTGCAGGATCTTTGATGGTTACGACGGGGGCCCCCT |
| | | TTGACTACTGGGGCCAAGGCACCACTCTCACAGTCTCCTCA |
| 34 | Nucleic acid sequence | GACATTGTGATGACCCAGTCTCACAAATTCATGTCCACATCAGTAG |
| | of light chain | GAGACAGGGTCAGCATCACCTGCAAGGCCAGTCAGGATGTGGGTAT |
| | variable domain of | TGCTGTTGCCTGGTATCAACAGAAACCAGGGCAATCTCCTAAATTT |
| | MAb1 | CTGATTTACTGGGCATCCACCCGGCACACTGGAGTCCCTGATCGCT |
| | | TCACAGGCAGTGGATCTGGGACAGATTTCACTCTCACCATTAGGAA |
| | | TGTGCAGTCTGAAGACTTGTCAGATTATTTCTGTCAGCAATATAGG |
| | | AGCTATCCTCTCACGTTCGGCTCGGGGACAAAGTTGGAACTAAAA |
| 35 | Linker sequence | GGGGS |
| 36 | Linker sequence | GGGGA |
| 37 | MAb4-YTE epitope 1 | KR |
| 38 | MAb4-YTE epitope 2 | KHNGMWRLVGITS |
| 39 | MAb4-YTE epitope 3 | YMDWILEKT |
| 40 | DX-2930 epitope 1 | HCFDGLPLQDVWR |
| 41 | DX-2930 epitope 2 | YKVSEGNH |
| 42 | DX-2930 epitope 3 | YKITQR |
| 43 | HCDR1 MAb 1, 2, 3, 4, | GYAFSSYWMN |
| | 5 (AbM) | |
| 44 | HCDR2 MAb 1, 2, 3, 4, | QIYPGDDDTN |
| | 5 (AbM) | |
| 45 | Sequence of Macaca | MILFKQATYFISLFATVSCGCLTQLYENAFFRGGDVASMYTPTAQH |
| | fascicularis (Crab- | CQMMCTFHPRCLLFSFLPASSINDMEKRFGCFLKDSVTGTLPKVRR |
| | eating macaque a/k/a | AGAISGHSLKQCGHQISACHRDIYKGIDMRGVNFNVSKVSSVEECQ |
| | Cynomolgus monkey) | KRCTNNIRCQFFSYATQTFHNAEYRNTCLLKHSPGGTPTTIKVLNN |
| | plasma kallikrein | VESGFSLKPCALSEIGCHMNIFQHLAFSDVDVARVLAPDAFVCRTI |
| | (Uniprot A0A2K5VTJ9-1) | CTYHPSCLFFTFYTNAWKIESQRNVCFLKTSESGTPSSSTPQENTT |
| | | SGYSLLTCKRTLPEPCHSKIYPGVDFGGEELNVTFVKGVNVCQETC |
| | | TKMIRCQFFTYSLLPEDCKEEKCKCFLRLSSDGSPTRITYGTQGSS |
| | | GYSLRLCNTGDSSVCTTKTSSRIVGGTNSSWGEWPWQVSLQVKLMA |
| | | QRHLCGGSLIGHQWVLTAAHCFDGLPLPDVWRIYSGILNLSDITKE |
| | | TPFSQIKEIIIHQNYRISEGNHDIALIKLQAPLNYTEFQKPICLPS |
| | | KGDTNTIYTNCWVTGWGFSKEKGEIQDILQKVNIPLVTNEECQKRY |
| | | QDYKITQRMVCAGYKEGGKDACKGDSGGPLACKHNGMWRLVGITSW |
| | | GEGCARREQPGVYTKVAEYMDWILEKTQSSDGNARMQAPA |
| 46 | Sequence of rabbit | MIPFRQAAYFVCLFTTVSCGCLTQLYKNAFFRGGDVTSMYSPNAQH |
| | plasma kallikrein | CQMMCTFHPRCLFFSFLPASSTNDAQKRFGCFLKDSVTGTLPRVHQ |
| | (Uniprot G1T127) | TNAISGHSLKQCGHQINACHRATYEGIDMRGTNFNISKVQSVEECQ |
| | | KQCTDNIHCQFFTYATQTFYNAEHLNNCILKKSATGTPSSIKLLEN |
| | | VVSGFSLKPCALSESGCHMNLFQHLAFSDVDVARVLTPDAFVCRTI |
| | | CTYHSNCLFFTFYTNEWDTESQRNACFLKTSESGTPSFPTPQENTI |
| | | SGYSLLTCKKTLPEPCHFKIYSGVDFGGEELNVTFVKGVTLCQETC |
| | | TKTIRCQFFTYSLLPEDCKGEKCRCSLRLSLDGSPTSITHGTQKSS |
| | | GYSLRLCKTENGSVCTTKINARIVGGSNSSRGEWPWQVSLQVKLAA |
| | | QSHVCGGSIIGHQWVLTAAHCFDGLPFPEIWRIYGGILYLSEVTKE |
| | | TAFSQIKEIIIHPKYKISETGHDIALIQLQAPLNDTDIQKPICLPS |
| | | KDDTNAIYTNCWVTGWGFTKEKGEIQNILQKANIPLVTNEECQKSY |
| | | RDHAITKQMVCAGYKEGGKDACKGDSGGPLVCKHNNIWLLVGITSW |
| | | GEGCARREQPGVYTKVAEYVDWILQKMQEKD |
| 47 | Sequence of rat plasma | MILFKQVGYFVSLFATVSCGCLSQLYANTFFRGGDLAAIYTPDAQH |
| | kallikrein (Uniprot | CQKMCTFHPRCLLFSFLAVSPTKETDKRFGCFMKESITGTLPRIHR |
| | P14272) | TGAISGHSLKQCGHQLSACHQDIYEGLDMRGSNFNISKTDSIEECQ |
| | | KLCTNNIHCQFFTYATKAFHRPEYRKSCLLKRSSSGTPTSIKPVDN |
| | | LVSGFSLKSCALSEIGCPMDIFQHFAFADLNVSQVVTPDAFVCRTV |
| | | CTFHPNCLFFTFYTNEWETESQRNVCFLKTSKSGRPSPPIIQENAV |
| | | SGYSLFTCRKARPEPCHFKIYSGVAFEGEELNATFVQGADACQETC |
| | | TKTIRCQFFTYSLLPQDCKAEGCKCSLRLSTDGSPTRITYEAQGSS |
| | | GYSLRLCKVVESSDCTTKINARIVGGTNSSLGEWPWQVSLQVKLVS |
| | | QNHMCGGSIIGRQWILTAAHCFDGIPYPDVWRIYGGILNLSEITNK |
| | | TPFSSIKELIIHQKYKMSEGSYDIALIKLQTPLNYTEFQKPICLPS |
| | | KADTNTIYTNCWVTGWGYTKERGETQNILQKATIPLVPNEECQKKY |
| | | RDYVITKQMICAGYKEGGIDACKGDSGGPLVCKHSGRWQLVGITSW |
| | | GEGCARKEQPGVYTKVAEYIDWILEKIQSSKERALETSPA |
| 48 | MAb4-YTE Heavy chain | CAGGTGCAGCTGGTGCAGTCTGGCGCCGAAGTGAAGAAACCTGGCT |
| | Nucleotide sequence | CCTCCGTGAAGGTGTCCTGCAAGGCCTCCGGCTATGCATTCAGTAG |
| | | CTACTGGATGAACTGGGTGAGGCAGGCTCCTGGACAGGGTCTTGAG |
| | | TGGATTGGACAGATTTATCCTGGAGATGACGATACTAACTACAATG |
| | | CCAAGTTCCAGGGGAGGGTCACCATCACCGTGGACAAATCCACAAC |
| | | TACCGCCTACATGGAACTCAGCAGCCTACGCTCTGAGGACACAGCC |
| | | GTCTATTTCTGTGCTGGCTCCCTGATGGTTACTACCGGCGCCCCTT |
| | | TTGACTACTGGGGCCAGGGAACCACGGTCACCGTCTCCTCAGCCTC |
| | | CACCAAGGGCCCATCGGTCTTCCCCCTGGCACCCTCTTCTAAGAGC |
| | | ACCTCTGGGGGCACAGCGGCCCTGGGCTGCCTGGTCAAGGACTACT |
| | | TCCCCGAACCGGTGACGGTGTCGTGGAACTCAGGCGCCCTGACCAG |
| | | CGGCGTGCACACCTTCCCGGCTGTCCTACAGTCCTCAGGACTCTAC |
| | | TCCCTCAGCAGCGTGGTGACCGTGCCCTCCAGCAGCTTGGGCACCC |
| | | AGACCTACATCTGCAACGTGAATCACAAGCCCAGCAACACCAAGGT |
| | | GGACAAGAGAGTTGAGCCCAAATCTTGTGACAAAACTCACACATGC |
| | | CCACCGTGCCCAGCACCTGAACTCCTGGGGGGACCGTCAGTCTTCC |
| | | TCTTCCCCCCAAAACCCAAGGACACCCTCTACATCACCCGGGAACC |
| | | TGAGGTCACATGCGTGGTGGTGGACGTGAGCCACGAAGACCCTGAG |
| | | GTCAAGTTCAACTGGTACGTGGACGGCGTGGAGGTGCATAATGCCA |
| | | AGACAAAGCCGAGAGAGGAGCAGTACAACAGCACGTACCGTGTGGT |
| | | CAGCGTCCTCACCGTCCTGCACCAGGACTGGCTGAATGGCAAGGAG |
| | | TACAAGTGCAAGGTCTCCAACAAAGCCCTCCCAGCCCCCATCGAGA |
| | | AAACCATCTCCAAAGCCAAAGGGCAGCCCCGAGAACCACAGGTGTA |
| | | CACCCTGCCCCCATCCCGGGAGGAGATGACCAAGAACCAGGTCAGC |
| | | CTGACCTGCCTGGTCAAAGGCTTCTATCCCAGCGACATCGCCGTGG |
| | | AGTGGGAGAGCAATGGGCAGCCGGAGAACAACTACAAGACCACGCC |
| | | TCCCGTGCTGGACTCCGACGGCTCCTTCTTCCTCTATTCCAAGCTC |
| | | ACCGTGGACAAGAGCAGGTGGCAGCAGGGGAACGTCTTCTCATGCT |
| | | CCGTGATGCATGAGGCTCTGCACAACCACTACACGCAGAAGAGCCT |
| | | CTCCCTGTCTCCCGGG |
| 49 | MAb4 Light Chain | GACATCCAGATGACCCAGTCTCCATCCTCCCTGTCTGCATCTGTAG |
| | Nucleotide sequence | GAGACAGAGTCACCATCACTTGCAAAGCAAGTCAGGACGTGGGAAT |
| | | CGCTGTCGCCTGGTATCAGCAGAAACCAGGGAAAGCCCCTAAGTTC |
| | | CTGATCTATTACGCATCCCACCGCGGATGGGGCGTGCCTGACAGGT |
| | | TCAGTGGCAGTGGATCTGGGACAGATTTCACTCTCACCATCAGCAG |
| | | TCTGCAACCTGAAGATTTTGCAACTTATTTCTGTCAACAGTACCGG |
| | | TCTTATCCCCTGACCTTTGGCCAAGGGACCAAGCTGGAAATCAAAC |
| | | GAACTGTGGCTGCACCATCTGTCTTCATCTTCCCGCCATCTGATGA |
| | | GCAGCTTAAGTCCGGAACTGCTAGCGTTGTGTGCCTGCTGAATAAC |
| | | TTCTATCCCAGAGAGGCCAAAGTACAGTGGAAGGTGGATAACGCCC |
| | | TCCAATCGGGAAACTCCCAGGAGAGTGTCACAGAGCAGGACAGCAA |
| | | GGACAGCACCTACAGCCTCAGCAGCACCCTGACGCTGAGCAAAGCA |
| | | GACTACGAGAAACACAAAGTCTACGCCTGCGAAGTCACCCATCAGG |
| | | GCCTGAGCTCGCCCGTCACAAAGAGCTTCAACAGGGGAGAGTGT |

1. An antibody, or antigen binding fragment thereof, that binds to plasma kallikrein comprising a heavy chain variable region (VH) comprising an HCDR1, an HCDR2, and an HCDR3; and a light chain variable region (VL) sequence comprising an LCDR1, an LCDR2, and an LCDR3, wherein:
a. (i) the HCDR1 comprises the amino acid sequence set forth in SEQ ID NO: 14, 23, 29, or 43; the HCDR2 comprises the amino acid sequence set forth in SEQ ID NO: 15, 17, 19, 24, 30, or 44; and the HCDR3 comprises the amino acid sequence set forth in SEQ ID NO: 16, 25, or 31; and
(ii) the LCDR1 comprises the amino acid sequence set forth in SEQ ID NO: 11, 20, or 27; the LCDR2 comprises the amino acid sequence set forth in SEQ ID NO: 12, 18, 21, or 26; and the LCDR3 comprises the amino acid sequence set forth in of SEQ ID NO: 13, 22, or 28;
b. (i) the HCDR1 comprises the amino acid sequence set forth in SEQ ID NO: 14; the HCDR2 comprises the amino acid sequence set forth in SEQ ID NO: 15, 17, or 19; and the HCDR3 comprises the amino acid sequence set forth in SEQ ID NO: 16; and
(ii) the LCDR1 comprises the amino acid sequence set forth in SEQ ID NO: 11; the LCDR2 comprises the amino acid sequence set forth in SEQ ID NO: 12 or 18; and the LCDR3 comprises the amino acid sequence set forth in of SEQ ID NO: 13;
c. (i) the HCDR1 comprises the amino acid sequence set forth in SEQ ID NO: 23; the HCDR2 comprises the amino acid sequence set forth in SEQ ID NO: 24; and the HCDR3 comprises the amino acid sequence set forth in SEQ ID NO: 25 and
(ii) the LCDR1 comprises the amino acid sequence set forth in SEQ ID NO: 20; the LCDR2 comprises the amino acid sequence set forth in SEQ ID NO: 21 or 26; and the LCDR3 comprises the amino acid sequence set forth in SEQ ID NO: 22;
d. (i) the HCDR1 comprises the amino acid sequence set forth in SEQ ID NO: 29; the HCDR2 comprises the amino acid sequence set forth in SEQ ID NO: 30; and the HCDR3 comprises the amino acid sequence set forth in SEQ ID NO: 31 and
(ii) the LCDR1 comprises the amino acid sequence set forth in SEQ ID NO: 27; the LCDR2 comprises the amino acid sequence set forth in SEQ ID NO: 21 or 26; and the LCDR3 comprises the amino acid sequence set forth in SEQ ID NO: 28; or
e. (i) the HCDR1 comprises the amino acid sequence set forth in SEQ ID NO: 43; the HCDR2 comprises the amino acid sequence set forth in SEQ ID NO: 44; and the HCDR3 comprises the amino acid sequence set forth in SEQ ID NO: 25 and
(ii) the LCDR1 comprises the amino acid sequence set forth in SEQ ID NO: 11; the LCDR2 comprises the amino acid sequence set forth in SEQ ID NO: 12 or 18 and the LCDR3 comprises the amino acid sequence set forth in SEQ ID NO: 28.

2-8. (canceled)

9. An antibody, or an antigen binding fragment thereof, comprising a heavy chain variable region (VH) comprising an HCDR1, an HCDR2, and an HCDR3; and a light chain variable region (VL) comprising an LCDR1, an LCDR2, and an LCDR3, wherein;
a. the HCDR1 comprises the amino acid sequence set forth in SEQ ID NO: 14 with up to 1, 2, or 3 amino acid substitutions to the amino acid sequence; the CDR2 comprises the amino acid sequence set forth in SEQ ID NO: 15, 17, or 19 with up to 1, 2, 3, 4, 5, 6, 7, 8, 9, 10, 11, 12, or 13 amino acid substitutions to the amino acid sequence; and the HCDR3 comprises the amino acid sequence set forth in SEQ ID NO: 16 with up to 1, 2, 3, 4, 5, 6, 7, 8, or 9 amino acid substitutions to the amino acid sequence; and
b. the LCDR1 comprises the amino acid sequence set forth in SEQ ID NO: 11 with up to 1, 2, 3, 4, 5, 6, or 7 amino acid substitutions to the amino acid sequence; the LCDR2 comprises the amino acid sequence set forth in SEQ ID NO: 12 or 18 with up to 1, 2, 3, or 4 amino acid substitutions to the amino acid sequence; and the LCDR3 comprises the amino acid sequence set forth in SEQ ID NO: 13 with up to 1, 2, 3, or 4 amino acid substitutions to the amino acid sequence.

10-12. (canceled)

13. The antibody or antigen binding fragment thereof of claim 1, wherein the VL region comprises the amino acid sequence set forth in SEQ ID NOs: 2, 4, or 6, and the VH region comprises the amino acid sequence set forth in SEQ ID NOs: 1, 3, or 5.

14-27. (canceled)

28. The antibody or antigen binding fragment thereof of claim 1, wherein the heavy chain comprises the amino acid sequence as set forth in SEQ ID NO: 10, 9, or 7, and the light chain comprises the amino acid sequence as set forth in SEQ ID NO: 8.

29-33. (canceled)

34. The antibody or antigen binding fragment thereof of claim 1, wherein the VL region comprises a sequence with at least 80%, 85%, 90%, 91%, 92%, 93%, 94%, 95%, 96%, 97%, 98%, or 99% percent homology to a sequence set forth in SEQ ID NOs: 6, 4, or 2, and the VH region comprises a sequence with at least 80%, 85%, 90%, 91%, 92%, 93%, 94%, 95%, 96%, 97%, 98%, or 99% percent homology to a sequence set forth in SEQ ID NOs: 1, 3, or 5.

35. The antibody or antigen binding fragment thereof of claim 1, wherein the heavy chain comprises a sequence with at least 80%, 85%, 90%, 91%, 92%, 93%, 94%, 95%, 96%, 97%, 98%, or 99% percent homology to the sequence as set forth in SEQ ID NO: 7, 9, or 10 and the light chain comprises a sequence with at least 80%, 85%, 90%, 91%, 92%, 93%, 94%, 95%, 96%, 97%, 98%, or 99% percent homology to the sequence as set forth in SEO ID NO: 8.

36-39. (canceled)

40. The antibody or antigen binding fragment thereof of claim 1, wherein the antibody or antigen binding fragment thereof binds to at least one of the following residues: K550, R551, K585, H586, N587, G588, M589, W590, R591, L592, V593, G594, I595, T596, S597, Y617, M618, D619, W620, I621, L622, E623, K624, or T625 of human plasma kallikrein having the amino acid sequence set forth in SEQ ID NO:32.

41-42. (canceled)

43. The antibody or antigen binding fragment thereof of claim 1, wherein the antibody has one or more of the following features:
a. a K_{D} that is substantially pH independent between pH 6.0-7.4 (e.g., as determined by SPR at 37° C. in a buffer containing 50 mM HEPES, 150 mM NaCl, 0.1 or 1.3 mM Ca²⁺, 1 mg/mL BSA, 0.02% Tween-20)
b. a K_{D} of between 0.1 nM and 5 nM for human plasma kallikrein (e.g., as determined by SPR at 37° C. in a buffer containing 50 mM HEPES, 150 mM NaCl, 0.1 or 1.3 mM Ca²⁺, 1 mg/mL BSA, 0.02% Tween-20, pH 6.0 or 7.4);
c. a K_{D} of between 0.1 nM and 5 nM for human plasma kallikrein and a K_{D} of between 250 nM and 2,000 nM for human prekallikrein (e.g., as determined by SPR at 37° C. in a buffer containing 50 mM HEPES, 150 mM NaCl, 0.1 or 1.3 mM Ca²⁺, 1 mg/mL BSA, 0.02% Tween-20, pH 6.0 or 7.4), and/or
d. a K_{D} of less than or equal to about 1, 2, 3, 4, or 5×10⁻⁹ M for human plasma kallikrein, as measured by surface plasmon resonance (SPR) assay.

44-49. (canceled)

50. The antibody or antigen binding fragment thereof of claim 1, wherein:
(i) the antibody is a monoclonal antibody;
(ii) the antibody is a humanized antibody;
(iii) the antibody is an intact or full length antibody; and/or
(iv) the CDR sequences are interposed between human or humanized framework sequences.

51-55. (canceled)

56. The antibody or antigen binding fragment thereof of claim 1, wherein the antibody comprises an IgG1 heavy chain constant region comprising an M252Y, an S254T, and a T256E mutation or an M428L and a N434S mutation as numbered according to the EU numbering index.

57-61. (canceled)

62. An isolated nucleic acid comprising a nucleotide sequence encoding the heavy chain or heavy chain variable region (VH) of claim 1.

63. An isolated nucleic acid comprising a nucleotide sequence encoding the light chain or light chain variable region (VL) of claim 1.

64. An expression vector comprising the nucleic acid of claim 62.

65. An expression vector comprising the nucleic acid of claim 63.

66. (canceled)

67. A host cell comprising the expression vector of claim 64.

68. (canceled)

69. A method of producing the antibody that binds plasma kallikrein or an antigen binding fragment thereof of claim 1, the method comprising:
(a) growing a host cell comprising (i) an expression vector comprising a nucleic acid encoding a polypeptide comprising the heavy chain variable region and a nucleic acid encoding a polypeptide comprising the light chain variable region, or (ii) an expression vector comprising a nucleic acid encoding a polypeptide comprising the heavy chain variable region and the light chain variable region under conditions so that the host cell expresses the polypeptide or polypeptides comprising the heavy chain variable region and the light chain variable region, thereby producing the antibody or the antigen-binding fragment of the antibody; and
(b) purifying the antibody or the antigen-binding fragment of the antibody.

70. A pharmaceutical composition comprising the antibody or antigen binding fragment of claim 1.

71-72. (canceled)

73. A method of treating a plasma kallikrein-mediated disorder in a subject, the method comprising administering an effective amount of an antibody of claim 1 or a pharmaceutical composition comprising the antibody or antigen binding fragment thereof to the subject.

74. The method of claim 73, wherein the disorder is hereditary angioedema, bradykinin dependent edema, diabetic macular edema, retinal edema, Factor XII-associated cold autoinflammatory syndrome (FACAS), rheumatoid arthritis, gout, intestinal bowel disease, oral mucositis, neuropathic pain, inflammatory pain, spinal stenosis-degenerative spine disease, arterial or venous thrombosis, post-operative ileus, aortic aneurysm, osteoarthritis, vasculitis, edema, cerebral edema, pulmonary embolism, stroke, clotting induced by ventricular assistance devices or stents, head trauma or peni-tumor brain edema, sepsis, acute middle cerebral artery (MCA) ischemic event (stroke), restenosis (e.g., after angioplasty), systemic lupus erythematosus nephritis, or burn injury.

75-77. (canceled)

78. A method of reducing bradykinin levels or inhibiting bradykinin production in a subject in need thereof comprising administering a therapeutically effective amount of an antibody or binding fragment thereof of claim 1 or a pharmaceutical composition comprising the antibody or antigen binding fragment thereof, to the subject.

79. A method of reducing or inhibiting plasma kallikrein activity in a subject in need thereof comprising administering a therapeutically effective amount of an antibody or binding fragment thereof of claim 1 or a pharmaceutical composition comprising the antibody or antigen binding fragment thereof, to the subject.

80. The method of 73, wherein the disease or disorder is hereditary angioedema.

81. (canceled)
